(19) United States
(12) Patent Application Publication
(10) Pub. No.: US 2023/0302083 A1
(43) Pub. Date: Sep. 28, 2023

(54) HIGHLY-NETWORKED CORONAVIRUS IMMUNOGEN COMPOSITION

(71) Applicants: The General Hospital Corporation, Boston, MA (US); Massachusetts Eye and Ear Infirmary, Boston, MA (US)

(72) Inventors: Gaurav GAIHA, Somerville, MA (US); Elizabeth Jeffries ROSSIN, Charlestown, MA (US)

(21) Appl. No.: 17/920,088

(22) PCT Filed: Apr. 20, 2021
(86) PCT No.: PCT/US2021/028245

§ 371 Date: Oct. 20, 2022

Related U.S. Application Data
(60) Provisional application No. 63/125,114, filed on Dec. 14, 2020.
(60) Provisional application No. 63/019,293, filed on May 2, 2020.
(60) Provisional application No. 63/012,565, filed on Apr. 20, 2020.

(51) Int. Cl. A61K 38/16; A61P 31/14; C12N 15/86; A61K 9/51

(52) U.S. Cl. CPC A61K 38/162; A61K 9/5123; A61P 31/14; C12N 15/86

(57) ABSTRACT

A method of preventing or treating COVID infection in a subject includes selecting two or more COVED CTL epitopes from a Coronavirus proteome that have a network score that meets a threshold value. An effective amount of a T cell immunogen composition and a pharmaceutically acceptable carrier is administered to the subject. The T cell immunogen composition includes the two or more selected Coronavirus CTL epitopes.

### SEQUENCE LISTING

The instant application contains a Sequence Listing which has been submitted electronically in ASCII format and is hereby incorporated by reference in its entirety. Said ASCII copy, created on Apr. 20, 2021, is named 51506-002WO5_Sequence_Listing 4_20_21_ST25 and is 21,439 bytes in size.

### CROSS-REFERENCE TO RELATED APPLICATIONS

This application claims benefit of the filing date of U.S. Application No. 63/012,565, filed on Apr. 20, 2020, U.S. Application No. 63/019,293, filed on May 2, 2020, and U.S. Application No. 63/125,114, filed on Dec. 14, 2020; the content of each of these priority applications is hereby incorporated by reference in its entirety.

### BACKGROUND

The development of an effective vaccine for the coronavirus disease of 2019 (COVID-19) is a critical global health priority. In order to combat this issue, there is a need for methods to systematically identify specific epitopes in the SARS-CoV-2 proteome, the etiologic agent of COVID-19, which are resistant to mutation and conserved across Coronaviruses. Targeting these epitopes would allow for persistent recognition and killing of cells infected by SARS-CoV-2 variants and other Coronaviruses by cytotoxic T lymphocytes in vivo.

### SUMMARY

Implementations described herein relate to highly networked coronavirus CTL epitopes and methods of identifying highly networked coronavirus CTL epitopes using a structure-based network analysis algorithm as well as to methods of preventing infection, reducing disease severity and treating a subject having or at risk of having a coronavirus infection through the use of T cell-based immunogens that incorporate the identified highly networked coronavirus CTL epitopes.

In certain implementations, a multi-epitope T cell immunogen composition comprising two or more highly networked coronavirus CTL epitopes is provided, wherein the two or more highly networked coronavirus CTL epitopes each have a network score of at least about 3.00, and wherein the highly networked Coronavirus CTL epitopes are restricted by one or more HLA alleles when expressed on the surface of a cell, e.g., an antigen presenting cell.

In other implementations, the two or more highly networked coronavirus CTL epitopes each having a network score of at least about 3.00 are selected from among the highly networked Coronavirus CTL epitopes that have high affinity for an HLA molecule (for example, those described in Table 5 or those described in Appendix 1 of U.S. provisional application nos. 63/012,565, 63/019,293, and 63/125,114, each of which is hereby incorporated by reference).

In other implementations, at least one of the two or more highly networked coronavirus CTL epitopes each having a network score of at least about 3.00 are selected from among the highly networked Coronavirus CTL epitope regions in Table 6 and/or Table 7.

In other implementations, at least one of the two or more highly networked coronavirus CTL epitopes each having a network score of at least about 3.00 is selected from among the highly networked Coronavirus CTL epitopes in Table 5 and is an epitope having an amino acid sequence of

AGEAANFCAL, ALNTLVKQL, AMPNMLRIM, APGTAVLRQW, APSASAFF,
APSASAFFGM, AQFAPSASA, AQVLSEMVM, ARTRSMWSF, AWPLIVTAL,
DRAMPNML, FCYMHHMEL, FELLHAPATV, FPQSAPHGV, FPQSAPHGVVF,
GEAANFCAL, GHLRIAGHHL, GNYQCGHYK, GTAVLRQW, GVDIAANTVIW,
GVFVSNGTHW, IAANTVIW, ILPVSMTK, IPTITQMNL, IPYNSVTSSI, IYQTSNFRV
KGIYQTSNF, KGIYQTSNFR, KLNDLCFTNV, KLNDLCFTNVY, KQASLNGVTL,
KRNVIPTITQM, KRVDFCGK, KRVDFCGKGY, KTSVDCTMY, KWADNNCYL,
LLKSAYENF, LLTLQQIEL, LLYDANYFL, LPVSMTKTSV, LRIAGHHL, LRQWLPTGTL,
LRQWLPTGTLL, MIAQYTSAL, MPILTLTRAL, MVMCGGSLY, MVMCGGSLYV,
MWSFNPETNIL, NASSSEAFL, NPLLYDANYFL, NSSPDDQIGY, NSSPDDQIGYY,
NVIPTITQM, PDDQIGYY, PGTAVLRQW, PLLTDEMIAQY, QFAPSASAF, QFAPSASAFF,
QPGQTFSVL, QPTESIVRF, QTFSVLACY, QVNGLTSIKW, QWLPTGTLL,
RGVYYPDKVF, RLFARTRSMW, RQLLFVVEV, RQWLPTGTL, RQWLPTGTLL,
RRGPEQTQGNF, RTRSMWSF, RVIHFGAGSDK, RVQPTESIVRF, SALNHTKKW,
SEMVMCGGSL, SEYTGNYQC, SFNPETNIL, SFNPETNILL, SIKNFKSVL,
SIKWADNNCY, SMWSFNPET, SPDDQIGYY, SSPDDQIGYY, SSPDDQIGYY, TEILPVSM,
TILTRPLL, TSNEVAVLY, TTLPVNVAF, VAPGTAVLRQW, VIPTITQMNL, VLNDILSRL,
VMCGGSLYV, VMCGGSLYVK, VNGLTSIKW, VPVVDSYY, VSMTKTSV,
VTANVNALL, VVNAANVYL, YDANYFLCW, YHLMSFPQSA, YLATALLTL,
YPKCDRAM, YQCGHYKHI, YQDVNCTEV, YRFNGIGV, YTGNYQCGHY,
YYPDKVFRSSV, YYSLLMPIL and/or YYSLLMPILTL.

In other implementations, a multi-epitope T cell immunogen composition comprising two or more highly networked Coronavirus CTL epitope variants is provided, wherein the two or more highly networked Coronavirus CTL epitope variants each have a network score of at least about 3.00, and at least one of the highly networked Coronavirus CTL epitope variants has at least about 65% to about 99% homology to a highly networked Coronavirus CTL epitope in Table 5. In other implementations, a method of preventing Coronavirus infection in a subject is provided. The method includes administering to the subject a prophylactically effective amount of a multi-epitope T cell immunogen composition comprising two or more highly networked Coronavirus CTL epitopes, wherein the two or more highly networked Coronavirus CTL epitopes each have a network score of at least about 3.00, and wherein the highly networked Coronavirus CTL epitopes are restricted by one or more HLA alleles and a pharmaceutically acceptable carrier, thereby preventing Coronavirus infection in the subject.

In certain implementations, a method of treating Coronavirus in a subject is provided. The method includes selecting two or more Coronavirus CTL epitopes from a Coronavirus proteome that have a network score that meets a threshold value. The network score for a given epitope can be determined by generating at least one network representing protein structure, calculating a set of network parameters, combining the network parameters to determine a network score for each amino acid residue in the protein structure, generating a network score for each of a plurality of epitopes as a weighted linear combination of the amino acid residues of the epitopes, and selecting two or more epitopes according to their network score. The method also includes administering to the subject a therapeutically effective amount of a T cell immunogen composition and a pharmaceutically acceptable carrier. The T cell immunogen composition includes the two or more selected Coronavirus CTL epitopes.

In other implementations, a method of preventing Coronavirus infection in a subject, or reducing the severity thereof, is provided. The method includes selecting two or more Coronavirus CTL epitopes from a Coronavirus proteome that have a network score that meets a threshold value. The network score for a given epitope can be determined by generating at least one network representing protein structure, calculating a set of network parameters, combining the network parameters to determine a network score for each amino acid residue in the protein structure, generating a network score for each of a plurality of epitopes as a weighted linear combination of the amino acid residues of the epitopes, and selecting two or more epitopes according to their network score. The method also includes administering to the subject a prophylactically effective amount of a T cell immunogen composition and a pharmaceutically acceptable carrier. The T cell immunogen composition includes the two or more selected Coronavirus CTL epitopes. Method of preventing Coronavirus infection in the subject, or reducing the severity thereof, include treatment of subjects infected with the P.1 Brazil SARS-CoV-2 variant, B.1.351 South African SARS-CoV-2 variant or B.1.17 United Kingdom SARS-CoV-2 variant.

In some implementations, a multi-epitope T cell immunogen composition including highly networked Coronavirus CTL epitopes

RGVYYPDKVFRSSV, KGIYQTSNFRVQPTESIVRF,
KLNDLCFTNVY, FELLHAPATV, TSNEVAVLYQDVNCTEV, TEILPVSMTKTSVDCTMY,
PLLTDEMIAQYTSAL, YRFNGIGV, ALNTLVKQLSSNFGAISSVLNDILSRL,
KRVDFCGKGYHLMSFPQSAPHGVVF, GVFVSNGTHW,
NPLLYDANYFLCWHTNCYDYCIPYNSVTSSI,
RLFARTRSMWSFNPETNILLNVPLHGTILTRPLLESELVIGAVILRGHLRIAGHHL,
NSSPDDQIGYY, and RRGPEQTQGNFGDQELIRQGTDYKHWPQIAQFAPSASAFFGM

is provided. In other implementations, a multi-epitope T cell immunogen including the sequences of the highly networked epitopes and the flanking sequences as shown in FIG. 11 is provided.

### BRIEF DESCRIPTION OF THE DRAWINGS

FIG. 1 is a flow chart illustrating an exemplary sequence of steps for selecting epitopes for a Coronavirus vaccine.

FIG. 2 is a schematic illustrating a structure- based network analysis in accordance with one implementation of the present invention. Atomic coordinates from PDB files (T4 Lysozyme, PDB: 2LZM) are utilized to determine inter-residue interactions using established 1) energy potentials and angle and distance thresholds and 2) distances between side-chain centers of mass. This edge-based representation of the protein is used for the application of the network centrality measures (second order degree centrality, summed node edge betweenness centrality and residue ligand proximity), as has been demonstrated in the network schematic for the central node (yellow). These values are then converted to Z-scores and summed to generate composite network scores for each amino acid residue in the protein, which is visually depicted by the size of the residue. The final output is a network-based representation of the protein on the Cα backbone of the PDB file.

FIG. 3 depicts structure-based network analysis of the SARS-CoV-2 proteome to identify amino acid residues conserved in lineage B and C coronaviruses. (A) Structure-based network analysis schematic for closed Spike trimer (PDB ID: 6VXX), including amino acid residues (nodes) and non-covalent interactions (edges). Edge width indicates interaction strength and node size indicates relative network scores. (B to D) Comparison of SARS-CoV-2 amino acid network scores (binned by network score: <0, 0-2, 2-4, and >4) with viral sequence entropy for SARS-CoV-2, sarbecoviruses (SARS-CoV-1/bat CoV) and MERS. (E) Alignment of SARS-CoV-2 network scores with viral sequence entropy values for SARS-CoV-2 in May 2020, SARS-CoV-2 in February 2021, sarbecoviruses (SARS-CoV-1/bat CoV) and MERS-CoV. Residues in blue indicate those with network scores greater than 4. Network scores of residues mutated in 501Y.V1 variant B.1.1.7 (red triangle), 501Y.V2 variant B.1.351 (green triangles) and 501Y.V3 variant P.1 are depicted in gray. Yellow boxes indicate new areas of sequence variation in SARS-CoV-2 that emerged between May 2020 and February 2021. Statistical comparisons were made using Mann-Whitney U test. For comparisons of more than two groups, Kruskal-Wallis test with Dunn’s pos hoc analyses were used. Calculated P values were as follows: *P < 0.05; **P < 0.01; ***P < 0.001; ****P < 0.0001.

FIG. 4 depicts structure-based network analysis of the SARS-CoV-2 proteome. (A,B) Network diagrams of SARS CoV-2 structural and accessory proteins and non-structural proteins. Node size indicates relative intra-protein network scores.

FIG. 5 depicts the correlation of SARS-CoV-2 network scores with SARS-CoV-1 and MERS-CoV. Scatter plots comparing SARS-CoV-2 network scores to (A) SARS-CoV-1 network scores and (B) MERS-CoV network scores. Correlations were calculated by Spearman’s rank correlation coefficient.

FIG. 6 depicts a spike pseudotyped lentiviral infectivity assay and comparison of network scores and Shannon entropy values for residues mutated in SARS-CoV-2 Spike protein. (A) List of matched pairs of networked and non-networked residues in the SARS-CoV-2 Spike proteins targeted for mutagenesis. (B) Comparison of network scores between networked residues and non-networked residues. (C and D) Comparison of Shannon entropy values between networked residues and non-networked residues in SARS-CoV-2 and the Sarbecovirus subgenus (SARSCoV-1/Bat CoV), respectively. (E) Flow cytometry plots showing %ZsGreen-positive 293T and 293T-ACE2 cells after 60h incubation with ZsGreen backbone lentiviruses pseudotyped with no Spike protein (delta Spike; gray), wild-type (WT) Spike protein (green) or VSV-G (black) envelope protein. Composite pseudotyped lentiviral infectivity data of (F) 293T or (G) 293T-ACE2 cells at five-fold and two-fold dilutions of neat stock virus preparations. Statistical comparisons were made using Mann-Whitney U test. Calculated P values were as follows: *P < 0.05; **P < 0.01; ***P <0.001; ****p < 0.0001.

FIG. 7 depicts mutation of highly networked residues in the viral Spike protein impairing pseudotyped lentiviral infectivity. (A) Location of networked (blue) and non-networked (red) residues in the closed (PDB ID: 6VXX) and open (PDB ID: 6VYB) conformations of the Spike protein that were mutated in pseudotyped lentiviral infectivity assay. (B) Flow cytometry plots showing %ZsGreen-positive 293T-ACE2 cells after 60h incubation with ZsGreen backbone lentiviruses pseudotyped with no Spike protein (delta Spike; gray), wild-type (WT) Spike (green), VSV-G (black) or mutant Spike proteins (dark blue, light blue and red). (C) Comparison of Spike pseudotyped lentiviral infectivity of 293T-ACE2 cells after mutation of networked residues with non-conservative mutations (N, dark blue), networked residues with conservative mutations (C, light blue) and non-networked residues with non-conservative mutations (N, red). Statistical analysis by one-way analysis of variance and Mann-Whitney U test. (D) Scatter plot of network score of the full Spike protein and average effect of mutation on monomeric RBD folding. Residues in blue indicate those with high network scores, but with low effect on monomeric RBD folding (V362, A363, C391, V524, C525). Correlations were calculated by Spearman’s rank correlation coefficient. (E) Location of highly networked residues with low effect on monomeric RBD folding (blue) within the RBD monomer (PDB ID: 6MOJ) and RBD-Distal S1 domain (PDB ID: 6VXX). (F) %ZSGreen-positive 293T-ACE2 cells after 60h incubation with WT Spike pseudotyped lentiviruses (green) and non-conservative (blue) or conservative (light blue) mutations to highly networked residues with low effect on monomeric RBD folding. (G) Scatter plot of network score of RBD and average effect of mutation on monomeric RBD folding. Residues in blue indicate those with high network scores, but with low effect on monomeric RBD folding. Correlations were calculated by Spearman’s rank correlation coefficient. For comparisons of more than two groups, Kruskal-Wallis test with Dunn’s post hoc analyses were used. Calculated P values were as follows: *P < 0.05; **P < 0.01; ***P < 0.001; ****P < 0.0001.

FIG. 8 depicts identification of highly networked CD8⁺ T cell epitopes by HLA class I-peptide stability assay. (A) Epitope prioritization pipeline for identification of highly networked CD8⁺ T cell epitopes in SARS-CoV-2. (B) Representative concentration-based stabilization of surface HLA-A*0301 following incubation with no peptide, immunodominant HIV HLA-A*0301 epitope RK9 (100 µM), predicted highly networked SARS-CoV-2 epitopes for HLA-A*0301 (100 µM) and B*08-restricted HIV epitope FL8 (100 µM). (C) Concentration-based HLA class I stabilization predicted highly networked SARS-CoV-2 CD8⁺ T cell epitopes for HLA-A*0301 (0.1-100 µM). The y axis depicts the anti-HLA MFI normalized to the highest value for each HLA class I allele (0-1). Immunodominant HIV HLA-A*0301 RK9 epitope is indicated in red. SARS-CoV-2 epitopes with at least 50% relative HLA-A*0301 stabilization in comparison to the immunodominant HIV RK9 epitope are indicated in dark blue, and those epitopes with less than 50% HLA-A*0301 stabilization in light blue. The non-HLA-A*03-restricted HIV epitope FL8 is indicated in light red. (D) Network-based depiction of A*03 RK11 (NSP16; PDB ID: 6W4H, Chain A) and A*03 KR10 (Spike; PDB ID: 6VXX). (E) Sequence alignments of A*03 RK11 and A*03 KR10 with the corresponding sequence for SARS-CoV-2 including the emerging variants, bat CoV RaTG13 and all coronaviruses known to infect humans. Putative HLA anchor residues in the SARS-CoV-2 epitope are underlined. (F) Fractions of highly networked CD8⁺ T cell epitopes in SARS-CoV-2 with ≤1 amino acid variant (blue), 2 variants (green), 3 variants (red), 4 variants (orange) and 5 variants (purple) in SARS-CoV-2 variants, bat CoV RaTG13 and all coronaviruses known to infect humans. (G) Comparison of HLA class I peptide stabilization for SARS-CoV-2 parental sequence epitopes and corresponding mutated epitopes in 501Y.V1 B.1.1.7 (red; A*02 VL9) or 501Y.V3 P.1 (yellow; A*01 SY10, A*01 NY10, A*01 NY11 and B*35 SY10) at 100 µM peptide concentration. Statistical comparison was made using Wilcoxon matched pairs test. (H) Comparison of the fraction of HLA*02-restricted highly networked (blue) and non-networked (red) epitope variants (Agerer et al., 2021) that achieve an allelic frequency greater than 0.1 or 0.9. Statistical comparisons of epitope variant frequencies were made using Fisher’s exact test Calculated P values were as follows: *P < 0.05; **P < 0.01; ***P < 0.001; ****P < 0.0001.

FIG. 9 depicts the concentration-based HLA class I-peptide stabilization of predicted SARS-CoV-2 CD8+ T cell epitopes. Concentration-based HLA class I stabilization of 311 predicted SARS-CoV-2 CD8+ T cell epitopes (0.1-100 uM) across 18 TAP-deficient mono-allelic HLA class I-expressing cell lines. The y axis depicts the anti-HLA MFI normalized to the known immunodominant HIV CD8+ T cell epitope (red) for each HLA class I allele. SARS-CoV-2 epitopes with >50% relative HLA class I stabilization to the HIV immunodominant epitope indicated in dark blue, and those with <50% relative stabilization are indicated in light blue.

FIG. 10 depicts CD8⁺ T cells from individuals with convalescent COVID-19 recognizing highly networked, HLA stabilizing CD8⁺ T cell epitopes derived from structural and accessory proteins. (A) Location of highly networked, HLA stabilizing CD8⁺ T cell epitopes in non-structural proteins (NSP; green) and structural proteins (SP; purple) across the SARS-CoV-2 proteome. (B) Representative IFN-γ ELISpot data for two pairs of healthy donors (HD) and COVID-19 patients following incubation with DMSO, anti-CD3/CD28 antibodies, CEF peptide pool, highly networked NSP peptide pool, highly networked SP peptide pool and combined NSP+SP peptide pool. The number of IFN-γ spot forming units (SFUs) is listed in the upper left of each well. A value of *** indicates that the response exceeded assay detection limits. (C) Magnitude of IFN-γ CD8⁺ T cell responses against CEF peptide pool in healthy donors (open, n = 20) and COVID-19 patients (filled, n = 30). Mild (filled circles, n = 21) and moderate-to-severe COVID-19 patients (filled diamonds, n = 9) are denoted. (D) Magnitude of IFN-γ CD8⁺ T cell responses against the highly networked SARS-CoV-2 NSP epitope pool (green), SP epitope pool (purple) and combined NSP+SP epitope pool (blue) in healthy donors (open, n = 20) and COVID-19 patients (filled, n = 30). (E) Magnitude of IFN-γ CD8⁺ T cell responses against the highly networked SARS-CoV-2 SP epitope pool (purple) in mild (n = 21) and moderate-to-severe COVID-19 patients (n = 9). (F) Comparison of the magnitude of IFN-γ CD8⁺ T cell responses to SP and NSP+SP peptide pools in COVID-19 SP peptide pool responders (n = 15). Statistical comparison was made using Wilcoxon matched pairs test. All other statistical comparisons were made using Mann-Whitney U test. Calculated P values were as follows: *P < 0.05; **P < 0.01; ***P < 0.001; ****P < 0.0001.

FIG. 11 is a chart depicting regions within SARS-CoV-2 structural and accessory proteins that are highly networked and which also harbor CD8+ T cell epitopes identified by HLA class I-peptide stability assay that achieved at least 50% relative HLA class I peptide stabilization in comparison to an immunodominant HIV epitope. The highly networked regions are underlined. Additional flanking amino acids are also included to assist with epitope processing.

FIG. 12 depicts the delivery of an alphavirus-based RNA replicon encoding immunogens composed of highly networked regions to HLA-A*02 transgenic mice by intra-muscular injection and the assessment of vaccine-induced T cell responses.

Appendix 1 (as described in U.S. Provisional Application Nos. 63/012,565, 63/019,293, and 63/125,114, each of which is hereby incorporated by reference) depicts all possible epitopes in SARS-CoV-2 for which epitope network scores could be calculated as determined according to the methods described herein.

The following Detailed Description, given by way of example, but not intended to limit the invention to specific embodiments described, may be understood in conjunction with the accompanying figures, incorporated herein by reference.

### DETAILED DESCRIPTION

All scientific and technical terms used in this application have meanings commonly used in the art unless otherwise specified. The definitions provided herein are to facilitate understanding of certain terms used frequently herein and are not meant to limit the scope of the application.

The articles “a” and “an” are used herein to refer to one or to more than one (i.e.,to at least one) of the grammatical object of the article. By way of example, “an element” means one element or more than one element. The terms “comprise,” “comprising,” “include,” “including,” “have,” and “having” are used in the inclusive, open sense, meaning that additional elements may be included. The terms “such as”, “e.g. ”, as used herein are non-limiting and are for illustrative purposes only. “Including” and “including but not limited to” are used interchangeably.

The term “or” as used herein should be understood to mean “and/or”, unless the context clearly indicates otherwise.

As used herein, the term “about” or “approximately” refers to a quantity, level, value, number, frequency, percentage, dimension, size, amount, weight or length that varies by as much as 15%, 10%, 9%, 8%, 7%, 6%, 5%, 4%, 3%, 2% or 1% to a reference quantity, level, value, number, frequency, percentage, dimension, size, amount, weight or length. In one embodiment, the term “about” or “approximately” refers a range of quantity, level, value, number, frequency, percentage, dimension, size, amount, weight or length ± 15%, ± 10%, ± 9%, ± 8%, ± 7%, ± 6%, ± 5%, ± 4%, ± 3%, ± 2%, or ± 1% about a reference quantity, level, value, number, frequency, percentage, dimension, size, amount, weight or length.

The term “vector” refers to a nucleic acid molecule capable of transporting another nucleic acid to which it has been linked. Preferred vectors are those capable of one or more of, autonomous replication and expression of nucleic acids to which they are linked. Vectors capable of directing the expression of genes to which they are operatively linked are referred to herein as “expression vectors”.

The term “variant” refers to a single or a grouping of sequences (e.g., in an amino acid sequence) that have undergone changes as referenced against a particular species or sub-populations within a particular species due to mutations, recombination/crossover or genetic drift. Examples of types of variants include, but are not limited to: single nucleotide polymorphisms (SNPs), copy number variations (CNVs), insertions/deletions (indels), single nucleotide variant (SNVs), multiple nucleotide variants (MNVs), inversions, etc. Variants may have homology to native (unmutated) amino acid sequences, including about 65% to about 99% homology to the amino acid sequence, about 75% to about 99% homology to the amino acid sequence, about 85% to about 99% homology to the amino acid sequence, about 90% to about 99% homology to the amino acid sequence, or about 95% to about 99% homology to the amino acid sequence.

As used herein, the terms “treatment,” “treating,” and the like, refer to obtaining a desired pharmacologic or physiologic effect. The effect may be therapeutic in terms of a partial or complete cure for a disease or an adverse effect attributable to the disease. “Treatment,” as used herein, covers any treatment of a disease in a mammal, particularly in a human, and can include inhibiting the disease or condition, i.e., arresting its development; and relieving the disease, i.e., causing regression of the disease. “Treatment,” as used herein, covers both prophylactic or preventive treatment (that prevents and/or slows the development of a targeted pathologic condition or disorder) and curative, therapeutic or disease-modifying treatment.

In certain embodiments, the term “treatment” can include inhibiting, attenuating or preventing the development or establishment of a COVID infection in a subject, e.g., by vaccination using a preventative vaccine including antigenic material described herein to stimulate a subject’s immune system to develop adaptive immunity to Coronavirus.

“Highly networked” refers to an epitope having a composite epitope network score of at least about 3.00. Highly networked is a quantitative description of an individual epitope based on the output from the structure-based network analysis method, which is derived from its position of the epitope within the three-dimensional structure of the Coronavirus protein. A network score greater than a score in the range of about 3.00, e.g., from about 2.90 to about 3.10 is encompassed by “highly networked” because the assignment of hydrogen atoms can differ slightly from one determination to another.

“Multi-networked” is a description of a nucleic acid or protein product (i.e. a T cell immunogen) that contains 2 or more highly networked epitopes.

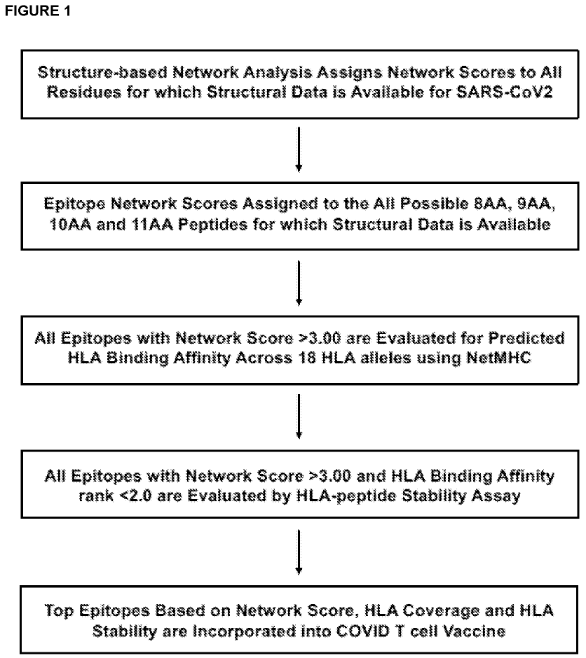

Implementations described herein relate to methods of identifying mutation resistant Coronavirus CTL epitopes using a structure-based network analysis algorithm as well as to methods of treating a subject in need thereof through the use of T cell-based immunogens that incorporate the identified mutation resistant Coronavirus CTL epitopes. It has been shown that a structure-based network analysis algorithm employing protein structure data and network theory metrics allows for the calculation of a network score for individual amino acid residues across the Coronavirus proteome thereby allowing for the identification of optimal mutation resistant cytotoxic T cell epitopes by summation of the individual amino acid residue network scores. Accordingly, an aspect of the invention relates to a method of identifying and selecting mutation resistant Coronavirus CTL epitopes for use in a COVID vaccine. FIG. 1 illustrates one example of a method 100 for selecting epitopes for a COVID vaccine. The method employs a structure-based network analysis, which utilizes protein structure data to quantify the topological importance of each amino acid residue to a protein’s tertiary and quaternary structure. The method 100 models the relationship between residue topology and mutational tolerance by focusing on interactions made by atoms unique to an amino acid’s identity. This was accomplished by using atomic level coordinate data from the Protein Data Bank to build networks comprising nodes, representing amino acid residues, and edges, representing non-covalent interactions between the amino acid residues. These inter-residue interactions were calculated between pairs of amino acids using energy potentials and established distance thresholds and summed to generate the protein network.

Using the network-based representation, an array of network centrality metrics, representing the relative importance of the various residues in a given network topology, are employed to provide a quantitative measure of the topological importance of individual amino acid residues through an assessment of their local connectivity to other residues, their involvement as bridges between higher order protein elements, such as secondary structure, tertiary and quaternary structure interfaces, and their proximity to known protein ligands. These metrics are integrated into a network score that quantifies the relative contribution of each amino acid residue to the protein’s topological structure.

At 102, at least one network representing protein structure is generated. An energetic approach, representing non-covalent interactions between individual atoms of amino acid residues, can be applied to generate one network. Non-covalent interactions considered in determining edge weights can include van der Waals interactions, hydrogen bonds, salt bridges, disulfide bonds, pi-pi interactions, pi-cation interactions, metal coordinated bonds and local hydrophobic packing. Each energetic protein network is then constructed by defining each individual amino acid residue within the protein structure as a node and defining weighted edges as the sum of all intermolecular bond energies between residues. Energies for each bond type were defined using previously established values in kJ/mol. The values for edges were then summed over the atoms in each amino acid residue to transform the edge list from a list of atom-atom interactions to a list of residue-residue interactions.

In an example implementation, the energetic network can be filtered to consider only those edges that are between terminal atoms to provide a second network focusing on residue-specific interactions. In this network, edges within the energetic network for which neither of the two participating atoms are a terminal atom are removed.

A centroid approach can be used to generate another network, representing the contribution of hydrophobic packing to protein folding. The centroid approach can be performed as an alternative or a supplement to the energetic approach Each centroid network, the side chain center of mass for each amino acid residue is calculated and bonds are defined based on a distance threshold cutoff between centroids of 8.5 angstroms. Centroid protein networks were then constructed by defining each amino acid residue as a node and defining edges as binary interactions that meet the defined 8.5 angstrom threshold for centroid-to-centroid distance. Edges to immediately neighboring amino acid residues were not included in either approach due to presence of covalent peptide bonds between these residues.

At 104, a set of network parameters are calculated. A first parameter represents the involvement of the residue in bridging different higher order protein structures. In the example implementation, higher order protein structures were identified in two ways, a classical method, for example as might be generated using the publicly available software tool Stride, and a random walk approach whereby tightly connected communities are identified and distinguished. One example of this is the Walktrap algorithm. For higher order structure filters, no edges were considered between residues within the same structural motif. The first parameter can be determined as a number of second order interactions between resides from different higher order structures, using either or both of the classical method and the random walk approach to identify the higher order structures.

A second order intermodular degree can be determined by determining, for each node, a number of nodes on different higher order structures within two degrees of separation of the network. This is referred to herein as the second order intermodular degree. In the example implementation, four separate values for the second order modulation degree can be determined for each node, using the three networks defined above and the two sets of secondary structure. Each second order intermodular degree value is obtained by summing, for each neighbor of the node associated with another secondary structure module, a number of edges associated with the neighboring node, with the links between the node and the secondary structure modules defined by different methods described above. If multimeric protein structure data is utilized, this metric can be considered for the multimer prior to normalization.

A first value represents the second order intermodular degree for each node in the energetic network using the classically defined secondary structure. A second value represents the second order intermodular degree for each node in the energetic network, filtered to include only edges between terminal atoms, using the classically defined secondary structure. A third value represents the second order intermodular degree for each node in the centroid network using the classically defined secondary structure. A fourth value represents the second order intermodular degree for each node in the centroid network using the secondary structure defined via the random walk approach. Each of the first, second, third, and fourth values can be standardized across all nodes to provide a standardized value, and a mean value across the first, second, third, and fourth values provides an overall value representing the second order intermodular degree, SD, for each node.

A node edge betweenness represents the frequency with which a node’s edges were utilized as a shortest path between all pairs of nodes in the network, weighted by edge weight. For each edge in the network bridging two nodes in different higher order structures, it is determined the number of times that the edge is used in a shortest path between a pair of nodes in the network, determined over all unique node pairs in the network as an edge betweenness. In the example implementation, the classically defined secondary structure is used to define the higher order structures. Once a value is determined for each edge, the edge betweenness for each edge associated with a node can be summed to provide a betweenness value for the node. In the example implementation, this is performed for each of the energetic network and the terminal filtered energetic network to provide two betweenness values, the values are standardized across all nodes, and then averaged to provide the final node edge betweenness value, NEB. If a multimeric version of the protein exists, then the maximum node edge betweenness is taken between the monomeric and multimeric conformations.

A Euclidean distance from centroid to ligand can be determined as the distance in angstroms of a residue’s centroid to the center of mass of the protein’s ligand. The centroid is defined as the center of mass of a residue’s sidechain, weighted by atomic weight. The center of mass of the ligand was calculated using all atoms. The resulting Euclidean distance from centroid to ligand, ED, is the distance between these two centers of mass, standardized across all residues.

At 106, the network parameters are combined to provide a network score for each node. In practice, each network parameter can be standardized across all nodes and combined in a weighted linear combination to provide a final network score. In the example implementation using the three network parameters described above, the final network score can be determined as:

$$S D + N E B - E D \qquad \text{Eq. 1}$$

At 108, a network score for each of a plurality of epitopes are determined as a weighted linear combination of the amino acid residues comprising the epitope. In the example implementation, the network score for each epitope is the sum of the network scores of the residues comprising the network. At 110, a set of epitopes are selected for use in the COVID vaccine based upon their network score. In one implementation, a set of epitopes with the highest network scores are selected. In another implementation, all epitopes have a network score meeting a threshold value can be utilized. It will be appreciated that the threshold value can vary with the implementation, but in the example implementation, a threshold value of 3.06 can be used, with all epitopes over that threshold being selected. Once identified and selected, delivery of selected multi-networked optimal Coronavirus CTL epitopes to a subject can be accomplished through the use of a T cell immunogen composition. Optimal mutation resistant multi-networked Coronavirus CTL epitopes selected in accordance with a method described herein can be incorporated into a T cell-based immunogen for use in generating an effective prophylactic and therapeutic T cell vaccine for COVID.

In certain implementations, a T cell immunogen composition can include two or more selected optimal Coronavirus CTL epitopes capable of inducing de novo cytotoxic T cell responses in the subject. The two or more highly networked coronavirus CTL epitopes each having a network score of at least about 3.00 can be selected from among the highly networked Coronavirus CTL epitopes that have high affinity based on computational predictions from NetMHCPan4.1 (http://www.cbs.dtu.dk/services/NetMHCpan/; Rank < 2.0) for an HLA molecule in Appendix 1 (as described in U.S. Provisional Application Nos. 63/012,565, 63/019,293, and 63/125,114, each of which is hereby incorporated by reference). Additionally, the at least one of the two or more optimal Coronavirus CTL epitopes each having a network score of at least about 3.0 can be selected from among the highly networked Coronavirus CTL epitopes in Table 5, including epitopes having an amino acid sequence of

AGEAANFCAL, ALNTLVKQL, AMPNMLRIM,
APGTAVLRQW, APSASAFF, APSASAFFGM, AQFAPSASA, AQVLSEMVM,
ARTRSMWSF, AWPLIVTAL, DRAMPNML, FCYMHHMEL, FELLHAPATV,
FPQSAPHGV, FPQSAPHGVVF, GEAANFCAL, GHLRIAGHHL, GNYQCGHYK,
GTAVLRQW, GVDIAANTVIW, GVFVSNGTHW, IAANTVIW, ILPVSMTK, IPTITQMNL,
IPYNSVTSSI, IYQTSNFRV, KGIYQTSNF, KGIYQTSNFR, KLNDLCFTNV,
KLNDLCFTNVY, KQASLNGVTL, KRNVIPTITQM, KRVDFCGK, KRVDFCGKGY,
KTSVDCTMY, KWADNNCYL, LLKSAYENF, LLTLQQIEL, LLYDANYFL,
LPVSMTKTSV, LRIAGHHL, LRQWLPTGTL, LRQWLPTGTLL, MIAQYTSAL,
MPILTLTRAL, MVMCGGSLY, MVMCGGSLYV, MWSFNPETNIL, NASSSEAFL,
NPLLYDANYFL, NSSPDDQIGY, NSSPDDQIGYY, NVIPTITQM, PDDQIGYY,
PGTAVLRQW, PLLTDEMIAQY, QFAPSASAF, QFAPSASAFF, QPGQTFSVL,
QPTESIVRF, QTFSVLACY, QVNGLTSIKW, QWLPTGTLL, RGVYYPDKVF,
RLFARTRSMW, RQLLFVVEV, RQWLPTGTL, RQWLPTGTLL, RRGPEQTQGNF,
RTRSMWSF, RVIHFGAGSDK, RVQPTESIVRF, SALNHTKKW, SEMVMCGGSL,
SEYTGNYQC, SFNPETNIL, SFNPETNILL, SIKNFKSVL, SIKWADNNCY, SMWSFNPET,
SPDDQIGYY, SSPDDQIGYY, SSPDDQIGYY, TEILPVSM, TILTRPLL, TSNEVAVLY,
TTLPVNVAF, VAPGTAVLRQW, VIPTITQMNL, VLNDILSRL, VMCGGSLYV,
VMCGGSLYVK, VNGLTSIKW, VPVVDSYY, VSMTKTSV, VTANVNALL,
VVNAANVYL, YDANYFLCW, YHLMSFPQSA, YLATALLTL, YPKCDRAM,
YQCGHYKHI, YQDVNCTEV, YRFNGIGV, YTGNYQCGHY, YYPDKVFRSSV,
YYSLLMPIL and/or YYSLLMPILTL.

Additionally, the at least one of the two or more optimal Coronavirus CTL epitopes each having a network score of at least about 3.0 can be selected from among the highly networked Coronavirus CTL epitopes regions in Appendices 3 and 4, including epitopes having an amino acid sequence of

ALNTLVKQL, APSASAFF, APSASAFFGM, AQFAPSASA, ARTRSMWSF,
AWPLIVTAL, FELLHAPATV, FPQSAPHGV, FPQSAPHGVVF, GHLRIAGHHL,
GVFVSNGTHW, ILPVSMTK, IPYNSVTSSI, IYQTSNFRV, KGIYQTSNF, KGIYQTSNFR,
KLNDLCFTNV, KLNDLCFTNVY, KRVDFCGK, KRVDFCGKGY, KTSVDCTMY,
LLYDANYFL, LPVSMTKTSV, LRIAGHHL, MIAQYTSAL, MPILTLTRAL,
MVMCGGSLY, MVMCGGSLYV, MWSFNPETNIL, NASSSEAFL, NPLLYDANYFL,
NSSPDDQIGY, NSSPDDQIGYY, PDDQIGYY, PLLTDEMIAQY, QFAPSASAF,
QFAPSASAFF, QPTESIVRF, RGVYYPDKVF, RLFARTRSMW, RRGPEQTQGNF,
RTRSMWSF, SFNPETNIL, SFNPETNILL, SMWSFNPET, SPDDQIGYY, SSPDDQIGYY,
SSPDDQIGYY, TEILPVSM, TILTRPLL, TSNEVAVLY, VLNDILSRL, VSMTKTSV,
YDANYFLCW, YHLMSFPQSA, YQDVNCTEV, YRFNGIGV or YYPDKVFRSSV.

T cell immunogen compositions can comprise any one of the compositions of Appendices 3 and 4 having amino acid sequences listed therein or variants thereof sharing at least about 65% to about 99% homology, or at least 75% to 85% homology.

Method of treating a subject for COVID infection are also provided. The methods can comprise administering to the subject a T cell immunogen composition including two or more optimal Coronavirus CTL epitopes, wherein the two of more optimal Coronavirus CTL epitopes have been identified and selected using a structure-based network analysis as described above. In some embodiments, the Coronavirus CTL epitopes are restricted on the surface of an antigen presenting cell by one or more HLA alleles.

In some implementations, a T cell immunogen composition for use in an COVID vaccine can include a recombinant vector including a nucleic acid sequence encoding two or more optimal CTL epitopes. Optimal CTL epitopes are highly networked, each having a network score of at least about 3.00 (e.g., from about 2.90 to about 3.10), when selected using the structure-based network analysis described herein. In some implementations, the optimal CTL epitopes selected using the structure-based network analysis described herein are CTL epitopes involved as either HLA anchor, TCR contact or peptide processing residues.

The Coronavirus CTL epitopes described herein are restricted by a particular HLA allele in vivo. “Restricted by” refers to the immunologic concept of HLA restriction, whereby certain epitopes are able to bind to specific HLA class I alleles and not others, and subsequently be recognized by T cells as a combined epitope-HLA complex. The phrase “the highly networked Coronavirus CTL epitopes are restricted by one or more HLA alleles” indicates that a potential highly networked T cell vaccine product could include multiple highly networked epitopes that bind to one HLA allele or several HLA alleles in vivo.

In other implementations, the optimal CTL epitope comprises two or more highly networked Coronavirus CTL epitope variants, wherein the two or more highly networked Coronavirus CTL epitope variants each have a network score of at least about 3.0, and the highly networked Coronavirus CTL epitope variant has at least about 65% to about 99% homology, or at least 75% to 85% homology, to a highly networked Coronavirus CTL epitope in Table 5.

The optimal Coronavirus CTL epitopes can be linked directly to one another with a linker. In some implementations, in some aspects, the linker is selected from the group consisting of: (1) consecutive glycine residues, at least 2, 3, 4, 5, 6, 7, 8, 9, or 10 residues in length; (2) consecutive alanine residues, at least 2, 3, 4, 5, 6, 7, 8, 9, or 10 residues in length; (3) two arginine residues (RR); (4) alanine, alanine, tyrosine (AAY); (5) a consensus sequence at least 2, 3, 4, 5, 6, 7, 8, 9, or 10 amino acid residues in length that is processed efficiently by a mammalian proteasome; and (6) one or more native sequences flanking the antigen derived from the cognate protein of origin and that is at least 2, 3, 4, 5, 6, 7, 8, 9, 10, 11, 12, 13, 14, 15, 16, 17, 18, 19, 20, or 2-20 amino acid residues in length. In some implementations, the linker comprises the sequence GPGPG.

The Coronavirus CTL epitopes described herein can be linked, operably or directly, to a separate or contiguous sequence that enhances the expression, stability, cell trafficking, processing and presentation, and/or immunogenicity of the epitope. The Coronavirus CTL sequence may include at least one of: an immunoglobulin signal sequence (e.g., IgK), a major histocompatibility class I sequence, lysosomal-associated membrane protein (LAMP)- 1, human dendritic cell lysosomal-associated membrane protein, and a major histocompatibility class II sequence.

In other implementations, at least one Coronavirus CTL epitope is linked, operably or directly, to a separate or contiguous sequence that enhances the expression, stability, cell trafficking, processing and presentation, and/or immunogenicity of the plurality. The separate or contiguous sequence can comprise at least one of: a ubiquitin sequence, a ubiquitin sequence modified to increase proteasome targeting (e.g., the ubiquitin sequence contains a Gly to Ala substitution at position 76 or Gly to Val substitution at position 76), an immunoglobulin signal sequence (e.g., IgK), a major histocompatibility class I sequence, lysosomal-associated membrane protein (LAMP)- 1, human dendritic cell lysosomal-associated membrane protein, and a major histocompatibility class II sequence; optionally wherein the ubiquitin sequence modified to increase proteasome targeting is A76 or V76.

The optimal Coronavirus CTL epitopes may be delivered to and expressed in a subject’s cells by incorporating a nucleic acid encoding a two or more optimal Coronavirus CTL epitopes into an expression vector. As used herein, “expression vector” refers to a vector that comprises a recombinant polynucleotide including expression control sequences operatively linked to a nucleotide sequence to be expressed. An expression vector comprises sufficient cis- acting elements for expression; other elements for expression can be supplied by the host cell or in an in vitro expression system. In some implementations, a recombinant expression vector can include additional immune-enhancer elements to increase epitope expression and/or de novo cytotoxic T cell responses in a subject. Immune-enhancer elements can include, but are not limited to, endoplasmic reticulum signal sequences (ERSS) to promote HLA class I presentation, sequences encoding a furin cleavage site (e.g., RRKR, RGRRKRS), and/or a universal T-helper epitope such as a pan HLA-DR epitope (PADRE).

Expression vectors include all those known in the art, such as cosmids, plasmids (e.g., naked or contained in liposomes), retrotransposons (e.g. piggyback, sleeping beauty), and viruses (e.g., lentiviruses, retroviruses, adenoviruses, and adeno-associated viruses) that can incorporate and deliver the recombinant polynucleotide.

Methods for producing viral vectors are known in the art. Typically, a disclosed virus is produced in a suitable host cell line using conventional techniques including culturing a transfected or infected host cell under suitable conditions so as to allow the production of infectious viral particles. Nucleic acids encoding viral genes and/or sequence(s) encoding two or more optimal Coronavirus CTL epitopes can be incorporated into plasmids and introduced into host cells through conventional transfection or transformation techniques. Exemplary suitable host cells for production of disclosed viruses include human cell lines such as HeLa, Hela-S3, HEK293, 911, A549, HER96, or PER-C6 cells. Specific production and purification conditions will vary depending upon the virus and the production system employed.

In some implementations, producer cells may be directly administered to a subject, however, in other implementations, following production, infectious viral particles are recovered from the culture and optionally purified. Typical purification steps may include plaque purification, centrifugation, e.g., cesium chloride gradient centrifugation, clarification, enzymatic treatment, e.g., benzonase or protease treatment, chromatographic steps, e.g., ion exchange chromatography or filtration steps.

In certain implementations, the expression vector is a viral vector. The term “virus” is used herein to refer any of the obligate intracellular parasites having no protein-synthesizing or energy-generating mechanism. Exemplary viral vectors include retroviral vectors (e.g., lentiviral vectors), adenoviral vectors, adeno-associated viral vectors, herpesviruses vectors, epstein-barr virus (EBV) vectors, polyomavirus vectors (e.g., simian vacuolating virus 40 (SV40) vectors), poxvirus vectors, and pseudotype virus vectors.

The virus may be an RNA virus (having a genome that is composed of RNA) or a DNA virus (having a genome composed of DNA). In certain implementations, the viral vector is a DNA virus vector. Exemplary DNA viruses include parvoviruses (e.g., adeno-associated viruses), adenoviruses, asfarviruses, herpesviruses (e.g., herpes simplex virus 1 and 2 (HSV-1 and HSV-2), epstein-barr virus (EBV), cytomegalovirus (CMV)), papillomoviruses (e.g., HPV), polyomaviruses (e.g., simian vacuolating virus 40 (SV40)), and poxviruses (e.g., vaccinia virus, cowpox virus, smallpox virus, fowlpox virus, sheeppox virus, myxoma virus). In certain implementations, the viral vector is a RNA virus vector. Exemplary RNA viruses include bunyaviruses (e.g., hantavirus), coronaviruses, ebolaviruses, flaviviruses (e.g., yellow fever virus, west nile virus, dengue virus), hepatitis viruses (e.g., hepatitis A virus, hepatitis C virus, hepatitis E virus), influenza viruses (e.g., influenza virus type A, influenza virus type B, influenza virus type C), measles virus, mumps virus, noroviruses (e.g., Norwalk virus), poliovirus, respiratory syncytial virus (RSV), retroviruses (e.g., human immunodeficiency virus-1 (HIV-1)) and toroviruses.

In certain implementations, the expression vector comprises a regulatory sequence or promoter operably linked to the nucleotide sequence encoding the two or more selected optimal Coronavirus CTL epitopes. The term “operably linked” refers to a linkage of polynucleotide elements in a functional relationship. A nucleic acid sequence is “operably linked” when it is placed into a functional relationship with another nucleic acid sequence. For instance, a promoter or enhancer is operably linked to a gene if it affects the transcription of the gene. Operably linked nucleotide sequences are typically contiguous. However, as enhancers generally function when separated from the promoter by several kilobases and intronic sequences may be of variable lengths, some polynucleotide elements may be operably linked but not directly flanked and may even function in trans from a different allele or chromosome.

Nucleic acid sequences encoding two or more selected optimal Coronavirus CTL epitopes preferably have strong promoters that are active in a variety of cell types. The promoters for eukaryotic nucleic acid sequences are typically present within the structural sequences encoding the two or more optimal Coronavirus CTL epitopes itself. Although there are elements which regulate transcriptional activity within the 5′ upstream region, the length of an active transcriptional unit may be considerably less than 500 base pairs.

Additional exemplary promoters which may be employed include, but are not limited to, the retroviral LTR, the SV40 promoter, the human cytomegalovirus (CMV) promoter, the U6 promoter, or any other promoter (e.g., cellular promoters such as eukaryotic cellular promoters including, but not limited to, the histone, pol III, and β-actin promoters). Other viral promoters which may be employed include, but are not limited to, adenovirus promoters, TK promoters, and B19 parvovirus promoters. The selection of a suitable promoter will be apparent to those skilled in the art from the teachings contained herein.

In certain implementations, an expression vector is an adeno-associated virus (AAV) vector. AAV is a small, nonenveloped icosahedral virus of the genus Dependoparvovirus and family Parvovirus. AAV has a single-stranded linear DNA genome of approximately 4.7 kb. AAV is capable of infecting both dividing and quiescent cells of several tissue types, with different AAV serotypes exhibiting different tissue tropism.

AAV includes numerous serologically distinguishable types including serotypes AAV-1 to AAV-12, as well as more than 100 serotypes from nonhuman primates (See, e.g., Srivastava (2008) J. Cell Biochem., 105(1): 17-24, and Gao et al. (2004) J. Virol., 78(12), 6381-6388). The serotype of the AAV vector used in the present invention can be selected by a skilled person in the art based on the efficiency of delivery, tissue tropism, and immunogenicity. For example, AAV-1, AAV-2, AAV-4, AAV-5, AAV-8, and AAV-9 can be used for delivery to the central nervous system; AAV-1, AAV-8, and AAV-9 can be used for delivery to the heart; AAV-2 can be used for delivery to the kidney; AAV-7, AAV-8, and AAV-9 can be used for delivery to the liver; AAV-4, AAV-5, AAV-6, AAV-9 can be used for delivery to the lung, AAV-8 can be used for delivery to the pancreas, AAV-2, AAV-5, and AAV-8 can be used for delivery to the photoreceptor cells; AAV-1, AAV-2, AAV-4, AAV-5, and AAV-8 can be used for delivery to the retinal pigment epithelium; AAV-1, AAV-6, AAV-7, AAV-8, and AAV-9 can be used for delivery to the skeletal muscle. In certain implementations, the AAV capsid protein comprises a sequence as disclosed in U.S. Pat. No. 7,198,951, such as, but not limited to, AAV-9 (SEQ ID NOs: 1-3 of U.S. Pat. No. 7,198,951), AAV-2 (SEQ ID NO: 4 of U.S. Pat. No. 7,198,951), AAV-1 (SEQ ID NO: 5 of U.S. Pat. No. 7,198,951), AAV-3 (SEQ ID NO: 6 of U.S. Pat. No. 7,198,951), and AAV-8 (SEQ ID NO: 7 of U.S. Pat. No. 7,198,951). AAV serotypes identified from rhesus monkeys, e.g., rh0.8, rh0.10, rh0.39, rh0.43, and rh0.74, are also contemplated in the instant invention. Besides the natural AAV serotypes, modified AAV capsids have been developed for improving efficiency of delivery, tissue tropism, and immunogenicity. Exemplary natural and modified AAV capsids are disclosed in U.S. Pat. Nos. 7,906,111, 9,493,788, and 7,198,951, and PCT Publication No. WO2017189964A2.

The wild-type AAV genome contains two 145 nucleotide inverted terminal repeats (ITRs), which contain signal sequences directing AAV replication, genome encapsidation and integration. In addition to the ITRs, three AAV promoters, p5, p19, and p40, drive expression of two open reading frames encoding rep and cap genes. Two rep promoters, coupled with differential splicing of the single AAV intron, result in the production of four rep proteins (Rep 78, Rep 68, Rep 52, and Rep 40) from the rep gene. Rep proteins are responsible for genomic replication. The Cap gene is expressed from the p40 promoter, and encodes three capsid proteins (VP1, VP2, and VP3) which are splice variants of the cap gene. These proteins form the capsid of the AAV particle.

Because the cis-acting signals for replication, encapsidation, and integration are contained within the ITRs, some or all of the 4.3 kb internal genome may be replaced with foreign DNA, for example, an expression cassette for an exogenous nucleic acid sequence of interest encoding two or more optimal Coronavirus CTL epitopes. Accordingly, in certain implementations, the AAV vector comprises a genome comprising an expression cassette for an exogenous nucleic acid sequence encoding two or more optimal Coronavirus CTL epitopes flanked by a 5′ ITR and a 3′ ITR. The ITRs may be derived from the same serotype as the capsid or a derivative thereof. Alternatively, the ITRs may be of a different serotype from the capsid, thereby generating a pseudotyped AAV. In certain implementations, the ITRs are derived from AAV-2. In certain implementations, the ITRs are derived from AAV-5. At least one of the ITRs may be modified to mutate or delete the terminal resolution site, thereby allowing production of a self-complementary AAV vector.

The rep and cap proteins can be provided in trans, for example, on a plasmid, to produce an AAV vector. A host cell line permissive of AAV replication must express the rep and cap genes, the ITR-flanked expression cassette, and helper functions provided by a helper virus, for example adenoviral genes E1a, E1b55K, E2a, E4orf6, and VA (Weitzman et al., Adeno-associated virus biology. Adeno-Associated Virus: Methods and Protocols, pp. 1-23, 2011). Methods for generating and purifying AAV vectors have been described in detail (See e.g., Mueller et al., (2012) Current Protocols in Microbiology, 14D.1.1-14D.1.21, Production and Discovery of Novel Recombinant Adeno-Associated Viral Vectors). Numerous cell types are suitable for producing AAV vectors, including HEK293 cells, COS cells, HeLa cells, BHK cells, Vero cells, as well as insect cells (See e.g. U.S. Pat. Nos. 6,156,303, 5,387,484, 5,741,683, 5,691,176, 5,688,676, and 8,163,543, U.S. Pat. Publication No. 20020081721, and PCT Publication Nos. WO00/47757, WO00/24916, and WO96/17947). AAV vectors are typically produced in these cell types by one plasmid containing the ITR-flanked expression cassette, and one or more additional plasmids providing the additional AAV and helper virus genes.

AAV of any serotype may be used in the present invention. Similarly, it is contemplated that any adenoviral type may be used, and a person of skill in the art will be able to identify AAV and adenoviral types suitable for the production of their desired recombinant AAV vector (rAAV). AAV particles may be purified, for example by affinity chromatography, iodixonal gradient, or CsCl gradient.

AAV vectors may have single-stranded genomes that are 4.7 kb in size, or are larger or smaller than 4.7 kb, including oversized genomes that are as large as 5.2 kb, or as small as 3.0 kb. Thus, where the exogenous gene of interest to be expressed from the AAV vector is small, the AAV genome may comprise a stuffer sequence. Further, vector genomes may be substantially self-complementary thereby allowing for rapid expression in the cell. In certain implementations, the genome of a self-complementary AAV vector comprises from 5′ to 3′: a 5′ ITR; a first nucleic acid sequence comprising a promoter and/or enhancer operably linked to a nucleic acid sequence encoding two or more optimal Coronavirus CTL epitopes; a modified ITR that does not have a functional terminal resolution site; a second nucleic acid sequence complementary or substantially complementary to the first nucleic acid sequence; and a 3′ ITR. AAV vectors containing genomes of all types are suitable for use in the method of the present invention. Non-limiting examples of AAV vectors include pAAV-MCS (Agilent Technologies), pAAVK-EF1α-MCS (System Bio Catalog # AAV502A-1), pAAVK-EF1α-MCS1-CMV-MCS2 (System Bio Catalog # AAV503A-1), pAAV-ZsGreen1 (Clontech Catalog #6231), pAAV-MCS2 (Addgene Plasmid #46954), AAV-Stuffer (Addgene Plasmid #106248), pAAVscCBPIGpluc (Addgene Plasmid #35645), AAVS1_Puro_PGK1_3xFLAG_Twin_Strep (Addgene Plasmid #68375), pAAV-RAM-d2TTA::TRE-MCS-WPRE-pA (Addgene Plasmid #63931), pAAV-UbC (Addgene Plasmid #62806), pAAVS1-P-MCS (Addgene Plasmid #80488), pAAV-Gateway (Addgene Plasmid #32671), pAAV-Puro_siKD (Addgene Plasmid #86695), pAAVS1-Nst-MCS (Addgene Plasmid #80487), pAAVS1-Nst-CAG-DEST (Addgene Plasmid #80489), pAAVS1-P-CAG-DEST (Addgene Plasmid #80490), pAAVf EnhCB-lacZnls (Addgene Plasmid #35642), and pAAVS1-shRNA (Addgene Plasmid #82697). These vectors can be modified to be suitable for therapeutic use. For example, an exogenous nucleic acid sequence of interest encoding two or more selected optimal Coronavirus CTL epitopes can be inserted in a multiple cloning site, and a selection marker (e.g., puro or a gene encoding a fluorescent protein) can be deleted or replaced with another (same or different) exogenous gene of interest. Further examples of AAV vectors are disclosed in U.S. Pat. Nos. 5,871,982, 6,270,996, 7,238,526, 6,943,019, 6,953,690, 9,150,882, and 8,298,818, U.S. Pat. Publication No. 2009/0087413, and PCT Publication Nos. WO2017075335A1, WO2017075338A2, and WO2017201258A1.

In certain implementations, the viral vector can be a retroviral vector. Examples of retroviral vectors include moloney murine leukemia virus vectors, spleen necrosis virus vectors, and vectors derived from retroviruses such as rous sarcoma virus, harvey sarcoma virus, avian leukosis virus, human immunodeficiency virus, myeloproliferative sarcoma virus, and mammary tumor virus. Retroviral vectors are useful as agents to mediate retroviral-mediated gene transfer into eukaryotic cells.

In certain implementations, the retroviral vector is a lentiviral vector. In certain implementations, the recombinant retroviral vector is a lentiviral vector including nucleic acids sequences encoding the two or more optimal epitopes.

Exemplary lentiviral vectors include vectors derived from human immunodeficiency virus-1 (HIV-1), human immunodeficiency virus-2 (HIV-2), simian immunodeficiency virus (SIV), feline immunodeficiency virus (FIV), bovine immunodeficiency virus (BIV), Jembrana Disease Virus (JDV), equine infectious anemia virus (EIAV), and caprine arthritis encephalitis virus (CAEV).

Retroviral vectors typically are constructed such that the majority of sequences coding for the structural genes of the virus are deleted and replaced by the gene(s) of interest. Most often, the structural genes (i.e., gag, pol, and env), are removed from the retroviral backbone using genetic engineering techniques known in the art. This may include digestion with the appropriate restriction endonuclease or, in some instances, with Bal 31 exonuclease to generate fragments containing appropriate portions of the packaging signal. Accordingly, a minimum retroviral vector comprises from 5′ to 3′: a 5′ long terminal repeat (LTR), a packaging signal, an optional exogenous promoter and/or enhancer, an exogenous gene of interest, and a 3′ LTR. If no exogenous promoter is provided, gene expression is driven by the 5′ LTR, which is a weak promoter and requires the presence of Tat to activate expression. The structural genes can be provided in separate vectors for manufacture of the lentivirus, rendering the produced virions replication-defective. Specifically, with respect to lentivirus, the packaging system may comprise a single packaging vector encoding the Gag, Pol, Rev, and Tat genes, and a third, separate vector encoding the envelope protein Env (usually VSV-G due to its wide infectivity). To improve the safety of the packaging system, the packaging vector can be split, expressing Rev from one vector, Gag and Pol from another vector. Tat can also be eliminated from the packaging system by using a retroviral vector comprising a chimeric 5′ LTR, wherein the U3 region of the 5′ LTR is replaced with a heterologous regulatory element.

These new genes can be incorporated into the proviral backbone in several general ways. The most straightforward constructions are ones in which the structural genes of the retrovirus are replaced by a single gene which then is transcribed under the control of the viral regulatory sequences within the LTR. Retroviral vectors have also been constructed which can introduce more than one gene into target cells. Usually, in such vectors one gene is under the regulatory control of the viral LTR, while the second gene is expressed either off a spliced message or is under the regulation of its own, internal promoter.

Accordingly, the new gene(s) are flanked by 5′ and 3′ LTRs, which serve to promote transcription and polyadenylation of the virion RNAs, respectively. The term “long terminal repeat” or “LTR” refers to domains of base pairs located at the ends of retroviral DNAs which, in their natural sequence context, are direct repeats and contain U3, R and U5 regions. LTRs generally provide functions fundamental to the expression of retroviral genes (e.g., promotion, initiation and polyadenylation of gene transcripts) and to viral replication. The LTR contains numerous regulatory signals including transcriptional control elements, polyadenylation signals, and sequences needed for replication and integration of the viral genome. The U3 region contains the enhancer and promoter elements. The U5 region is the sequence between the primer binding site and the R region and contains the polyadenylation sequence. The R (repeat) region is flanked by the U3 and U5 regions. In certain implementations, the R region comprises a transactivation response (TAR) genetic element, which interacts with the trans-activator (tat) genetic element to enhance viral replication. This element is not required in implementations wherein the U3 region of the 5′ LTR is replaced by a heterologous promoter.

In certain implementations, the retroviral vector comprises a modified 5′ LTR and/or 3′ LTR. Modifications of the 3′ LTR are often made to improve the safety of lentiviral or retroviral systems by rendering viruses replication-defective. In specific implementations, the retroviral vector is a self-inactivating (SIN) vector. As used herein, a SIN retroviral vector refers to a replication-defective retroviral vector in which the 3′ LTR U3 region has been modified (e.g., by deletion or substitution) to prevent viral transcription beyond the first round of viral replication. This is because the 3′ LTR U3 region is used as a template for the 5′ LTR U3 region during viral replication and, thus, the viral transcript cannot be made without the U3 enhancer-promoter. In a further implementation, the 3′ LTR is modified such that the U5 region is replaced, for example, with an ideal polyadenylation sequence. It should be noted that modifications to the LTRs such as modifications to the 3′ LTR, the 5′ LTR, or both 3′ and 5′ LTRs, are also included in the invention.

In certain implementations, the U3 region of the 5′ LTR is replaced with a heterologous promoter to drive transcription of the viral genome during production of viral particles. Examples of heterologous promoters which can be used include, for example, viral simian virus 40 (SV40) (e.g., early or late), cytomegalovirus (CMV) (e.g., immediate early), Moloney murine leukemia virus (MoML V), Rous sarcoma virus (RSV), and herpes simplex virus (HSV) (thymidine kinase) promoters. Typical promoters are able to drive high levels of transcription in a Tat-independent manner. This replacement reduces the possibility of recombination to generate replication-competent virus, because there is no complete U3 sequence in the virus production system. Adjacent to the 5′ LTR are sequences necessary for reverse transcription of the genome and for efficient packaging of viral RNA into particles (the Psi site). As used herein, the term “packaging signal” or “packaging sequence” refers to sequences located within the retroviral genome which are required for encapsidation of retroviral RNA strands during viral particle formation (see e.g., Clever et al., 1995 J. Virology, 69(4):2101-09). The packaging signal may be a minimal packaging signal (also referred to as the psi [Ψ] sequence) needed for encapsidation of the viral genome.

In certain implementations, the retroviral vector (e.g., lentiviral vector) further comprises a FLAP. As used herein, the term “FLAP” refers to a nucleic acid whose sequence includes the central polypurine tract and central termination sequences (cPPT and CTS) of a retrovirus, e.g., HIV-1 or HIV-2. Suitable FLAP elements are described in U.S. Pat. No. 6,682,907 and in Zennou et al. (2000) Cell 101:173. During reverse transcription, central initiation of the plus-strand DNA at the cPPT and central termination at the CTS lead to the formation of a three-stranded DNA structure: a central DNA flap. While not wishing to be bound by any theory, the DNA flap may act as a cis-active determinant of lentiviral genome nuclear import and/or may increase the titer of the virus. In particular implementations, the retroviral vector backbones comprise one or more FLAP elements upstream or downstream of the heterologous nucleic acid sequence of interest in the vectors. For example, in particular implementations, a transfer plasmid includes a FLAP element. In one implementation, a vector of the invention comprises a FLAP element isolated from HIV-1.

In certain implementations, the retroviral vector (e.g., lentiviral vector) further comprises an export element. In one implementation, retroviral vectors comprise one or more export elements. The term “export element” refers to a cis-acting post-transcriptional regulatory element which regulates the transport of an RNA transcript from the nucleus to the cytoplasm of a cell. Examples of RNA export elements include, but are not limited to, the human immunodeficiency virus (HIV) RRE (see e.g., Cullen et al., (1991) J. Virol. 65: 1053; and Cullen et al., (1991) Cell 58: 423) and the hepatitis B virus post-transcriptional regulatory element (HPRE). Generally, the RNA export element is placed within the 3′ UTR of a gene, and can be inserted as one or multiple copies.

In certain implementations, the retroviral vector (e.g., lentiviral vector) further comprises a posttranscriptional regulatory element. A variety of posttranscriptional regulatory elements can increase expression of a heterologous nucleic acid, e.g., woodchuck hepatitis virus posttranscriptional regulatory element (WPRE; see Zufferey et al., (1999) J. Virol., 73:2886); the posttranscriptional regulatory element present in hepatitis B virus (HPRE) (Huang et al., Mol. Cell. Biol., 5:3864); and the like (Liu et al., (1995), Genes Dev., 9:1766). The posttranscriptional regulatory element is generally positioned at the 3′ end the heterologous nucleic acid sequence. This configuration results in synthesis of an mRNA transcript whose 5′ portion comprises the heterologous nucleic acid coding sequences and whose 3′ portion comprises the posttranscriptional regulatory element sequence. In certain implementations, vectors of the invention lack or do not comprise a posttranscriptional regulatory element such as a WPRE or HPRE, because in some instances these elements increase the risk of cellular transformation and/or do not substantially or significantly increase the amount of mRNA transcript or increase mRNA stability. Therefore, in certain implementations, vectors of the invention lack or do not comprise a WPRE or HPRE as an added safety measure.

Elements directing the efficient termination and polyadenylation of the heterologous nucleic acid transcripts increase heterologous gene expression. Transcription termination signals are generally found downstream of the polyadenylation signal. Accordingly, in certain implementations, the retroviral vector (e.g., lentiviral vector) further comprises a polyadenylation signal. The term “polyadenylation signal” or “polyadenylation sequence” as used herein denotes a DNA sequence which directs both the termination and polyadenylation of the nascent RNA transcript by RNA polymerase H. Efficient polyadenylation of the recombinant transcript is desirable as transcripts lacking a polyadenylation signal are unstable and are rapidly degraded. Illustrative examples of polyadenylation signals that can be used in a vector of the invention, includes an ideal polyadenylation sequence (e.g., AATAAA, ATTAAA AGTAAA), a bovine growth hormone polyadenylation sequence (BGHpA), a rabbit β-globin polyadenylation sequence (rβgpA), or another suitable heterologous or endogenous polyadenylation sequence known in the art.

In certain implementations, a retroviral vector further comprises an insulator element. Insulator elements may contribute to protecting retrovirus-expressed sequences, e.g., therapeutic nucleic acid sequences, from integration site effects, which may be mediated by cis-acting elements present in genomic DNA and lead to deregulated expression of transferred sequences (i.e., position effect; see, e.g., Burgess-Beusse et al., (2002) Proc. Natl. Acad. Sci., USA, 99:16433; and Zhan et al., 2001, Hum. Genet., 109:471). In certain implementations, the retroviral vector comprises an insulator element in one or both LTRs or elsewhere in the region of the vector that integrates into the cellular genome. Suitable insulators for use in the invention include, but are not limited to, the chicken β-globin insulator (see Chung et al., (1993). Cell 74:505; Chung et al., (1997) Proc. Natl. Acad. Sci., USA 94:575; and Bell et al., 1999. Cell 98:387). Examples of insulator elements include, but are not limited to, an insulator from a β-globin locus, such as chicken HS4.

Non-limiting examples of lentiviral vectors include pL VX-EF1alpha-AcGFP1-C1 (Clontech Catalog #631984), pL VX-EF1alpha-IRES-mCherry (Clontech Catalog #631987), pLVX-Puro (Clontech Catalog #632159), pLVX-IRES-Puro (Clontech Catalog #632186), pLenti6/V5-DEST™ (Thermo Fisher), pLenti6.2/V5-DEST™ (Thermo Fisher), pLKO.1 (Plasmid #10878 at Addgene), pLKO.3G (Plasmid #14748 at Addgene), pSico (Plasmid #11578 at Addgene), pLJM1-EGFP (Plasmid #19319 at Addgene), FUGW (Plasmid #14883 at Addgene), pLVTHM (Plasmid #12247 at Addgene), pLVUT-tTR-KRAB (Plasmid #11651 at Addgene), pLL3.7 (Plasmid #11795 at Addgene), pLB (Plasmid #11619 at Addgene), pWPXL (Plasmid #12257 at Addgene), pWPI (Plasmid #12254 at Addgene), EF.CMV.RFP (Plasmid #17619 at Addgene), pLenti CMV Puro DEST (Plasmid #17452 at Addgene), pLenti-puro (Plasmid #39481 at Addgene), pULTRA (Plasmid #24129 at Addgene), pLX301 (Plasmid #25895 at Addgene), pHIV-EGFP (Plasmid #21373 at Addgene), pLV-mCherry (Plasmid #36084 at Addgene), pLionII (Plasmid #1730 at Addgene), pInducer10-mir-RUP-PheS (Plasmid #44011 at Addgene). These vectors can be modified to be suitable for therapeutic use. For example, a selection marker (e.g., puro, EGFP, or mCherry) can be deleted or replaced with a second exogenous nucleic acid sequence of interest. Further examples of lentiviral vectors are disclosed in U.S. Pat. Nos. 7,629,153, 7,198,950, 8,329,462, 6,863,884, 6,682,907, 7,745,179, 7,250,299, 5,994,136, 6,287,814, 6,013,516, 6,797,512, 6,544,771, 5,834,256, 6,958,226, 6,207,455, 6,531,123, and 6,352,694, and PCT Publication No. WO2017/091786.

In some implementations, the viral vector can be an adenoviral vector. Adenoviruses are medium-sized (90-100 nm), non-enveloped (naked), icosahedral viruses composed of a nucleocapsid and a double-stranded linear DNA genome. The term “adenovirus” refers to any virus in the genus Adenoviridiae including, but not limited to, human, bovine, ovine, equine, canine, porcine, murine, and simian adenovirus subgenera. Typically, an adenoviral vector is generated by introducing one or more mutations (e.g., a deletion, insertion, or substitution) into the adenoviral genome of the adenovirus so as to accommodate the insertion of a non-native nucleic acid sequence, for example, for gene transfer, into the adenovirus.

A human adenovirus can be used as the source of the adenoviral genome for the adenoviral vector. For instance, an adenovirus can be of subgroup A (e.g., serotypes 12, 18, and 31), subgroup B (e.g., serotypes 3, 7, 11, 14, 16, 21, 34, 35, and 50), subgroup C (e.g., serotypes 1, 2, 5, and 6), subgroup D (e.g., serotypes 8, 9, 10, 13, 15, 17, 19, 20, 22-30, 32, 33, 36-39, and 42-48), subgroup E (e.g., serotype 4), subgroup F (e.g., serotypes 40 and 41 ), an unclassified serogroup (e.g., serotypes 49 and 51), or any other adenoviral serogroup or serotype. In an exemplary implementation, the adenovirus vector is a serotype 5 adenovirus vector

Adenoviral serotypes 1 through 51 are available from the American Type Culture Collection (ATCC, Manassas, Virginia). Non-group C adenoviral vectors, methods of producing non-group C adenoviral vectors, and methods of using non- group C adenoviral vectors are disclosed in, for example, U.S. Pat. Nos. 5,801,030, 5,837,511, and 5,849,561, and PCT Publication Nos. WO1997/012986 and WO1998/053087.

Non-human adenovirus (e.g., ape, simian, avian, canine, ovine, or bovine adenoviruses) can be used to generate the adenoviral vector (i.e., as a source of the adenoviral genome for the adenoviral vector). For example, the adenoviral vector can be based on a simian adenovirus, including both new world and old world monkeys (see, e.g., Virus Taxonomy: VHIth Report of the International Committee on Taxonomy of Viruses (2005)). A phylogeny analysis of adenoviruses that infect primates is disclosed in, e.g., Roy et al. (2009) PLoS Pathog. 5(7):e1000503. A gorilla adenovirus can be used as the source of the adenoviral genome for the adenoviral vector. Gorilla adenoviruses and adenoviral vectors are described in, e.g., PCT Publication Nos.WO2013/052799, WO2013/052811, and WO2013/052832. The adenoviral vector can also comprise a combination of subtypes and thereby be a “chimeric” adenoviral vector.

The adenoviral vector can be replication-competent, conditionally replication- competent, or replication-deficient. A replication-competent adenoviral vector can replicate in typical host cells, i.e., cells typically capable of being infected by an adenovirus. A conditionally-replicating adenoviral vector is an adenoviral vector that has been engineered to replicate under predetermined conditions. For example, replication-essential gene functions, e.g., gene functions encoded by the adenoviral early regions, can be operably linked to an inducible, repressible, or tissue-specific transcription control sequence, e.g., a promoter. Conditionally-replicating adenoviral vectors are further described in U.S. Pat. No. 5,998,205. A replication-deficient adenoviral vector is an adenoviral vector that requires complementation of one or more gene functions or regions of the adenoviral genome that are required for replication, as a result of, for example, a deficiency in one or more replication- essential gene function or regions, such that the adenoviral vector does not replicate in typical host cells, especially those in a human to be infected by the adenoviral vector.

The adenoviral vector can be replication-deficient, such that the replication- deficient adenoviral vector requires complementation of at least one replication-essential gene function of one or more regions of the adenoviral genome for propagation (e.g., to form adenoviral vector particles). The adenoviral vector can be deficient in one or more replication-essential gene functions of only the early regions (i.e., E1-E4 regions) of the adenoviral genome, only the late regions (i.e., L1-L5 regions) of the adenoviral genome, both the early and late regions of the adenoviral genome, or all adenoviral genes (i.e., a high capacity adenovector (HC-Ad)). See, e.g., Morsy et al. (1998) Proc. Natl. Acad. Sci. USA 95: 965-976, Chen et al. (1997) Proc. Natl. Acad. Sci. USA 94: 1645-1650, and Kochanek et al. (1999) Hum. Gene Ther. 10(15):2451-9. Examples of replication-deficient adenoviral vectors are disclosed in U.S. Pat. Nos. 5,837,511, 5,851,806, 5,994,106, 6,127,175, 6,482,616, and 7,195,896, and PCT Publication Nos. WO1994/028152, WO1995/002697, WO1995/016772, WO1995/034671, WO1996/022378, WO 1997/012986, WO 1997/021826, and WO2003/022311.

The replication-deficient adenoviral vector of the invention can be produced in complementing cell lines that provide gene functions not present in the replication-deficient adenoviral vector, but required for viral propagation, at appropriate levels in order to generate high titers of viral vector stock. Such complementing cell lines are known and include, but are not limited to, 293 cells (described in, e.g., Graham et al. (1977) J. Gen. Virol. 36: 59-72), PER.C6 cells (described in, e.g., PCT Publication No. WO1997/000326, and U.S. Pat. Nos. 5,994,128 and 6,033,908), and 293-ORF6 cells (described in, e.g., PCT Publication No. WO1995/034671 and Brough et al. (1997) J. Virol. 71: 9206-9213). Other suitable complementing cell lines to produce the replication-deficient adenoviral vector of the invention include complementing cells that have been generated to propagate adenoviral vectors encoding transgenes whose expression inhibits viral growth in host cells (see, e.g., U.S. Pat. Publication No. 2008/0233650). Additional suitable complementing cells are described in, for example, U.S. Pat. Nos. 6,677,156 and 6,682,929, and PCT Publication No. WO2003/020879. Formulations for adenoviral vector-containing compositions are further described in, for example, U.S. Pat. Nos. 6,225,289, and 6,514,943, and PCT Publication No. WO2000/034444.

Additional exemplary adenoviral vectors, and/or methods for making or propagating adenoviral vectors are described in U.S. Pat. Nos. 5,559,099, 5,837,511, 5,846,782, 5,851,806, 5,994,106, 5,994,128, 5,965,541, 5,981,225, 6,040,174, 6,020,191, 6,083,716, 6,113,913, 6,303,362, 7,067,310, and 9,073,980.

Commercially available adenoviral vector systems include the ViraPower™ Adenoviral Expression System available from Thermo Fisher Scientific, the AdEasy™ adenoviral vector system available from Agilent Technologies, and the Adeno-X™ Expression System 3 available from Takara Bio USA, Inc.

In certain implementations, the viral vector can be a Herpes Simplex Virus plasmid vector. Herpes simplex virus type-1 (HSV-1) has been demonstrated as a potential useful gene delivery vector system for gene therapy. HSV-1 vectors have been used for transfer of genes to muscle, and have been used for murine brain tumor treatment. Helper virus dependent mini-viral vectors have been developed for easier operation and their capacity for larger insertion (up to 140 kb). Replication incompetent HSV amplicons have been constructed in the art. These HSV amplicons contain large deletions of the HSV genome to provide space for insertion of exogenous DNA. Typically, they comprise the HSV-1 packaging site, the HSV-1 “ori S” replication site and the IE ⅘ promoter sequence. These virions are dependent on a helper virus for propagation.

In some implementations, the recombinant vector is a Vaccinia vector. Vaccinia are recombinant vaccines typically are used as vectors for expression of foreign genes within a host, in order to generate an in vivo immune response. In certain implementations, a Vaccinia vector for use in an immunogen composition described herein is a highly attenuated strain of a Vaccinia virus, such as Modified Vaccinia Ankara (MV A) virus. MV A can encode more than one foreign antigen and thus can effectively function as a multivalent vaccine. In animal models, MVA vector vaccines have been found to have intrinsic adjuvant capacities and be immunogenic and protective against various infectious agents including immunodeficiency viruses. Compared to replicating Vaccinia viruses, MVA provides similar or higher levels of recombinant gene expression even in non-permissive cells.

In some implementations, the recombinant vector can include messenger RNA (mRNA). One advantage of mRNA is that mRNA vaccines are capable of inducing a balanced immune response including both cellular and humoral immunity. In addition, mRNA vaccines can be designed to be self-adjuvanting. Alternatively, mRNA vaccines can be supplemented with one or more additional adjuvant molecules such as additional mRNAs encoding auxiliary adjuvant molecules.

Functional synthetic mRNA may be obtained by in vitro transcription of a cDNA template, typically plasmid DNA (pDNA), using a bacteriophage RNA polymerase. Synthetic mRNA for use in an mRNA vector immunogen composition described herein can include a protein-encoding open reading frame (ORF) flanked at the minimum by two elements essential for the function of mature eukaryotic mRNA: a “cap,” i.e., a 7-methyl-guanosine residue joined to the 5′-end via a 5′-5′ triphosphate, and a poly(A) tail at the 3′-end.

Therefore, in some implementations, a pDNA template can include a bacteriophage promoter, an ORF, optionally a poly(d(A/T)) sequence transcribed into poly(A) and a unique restriction site for linearization of the plasmid to ensure defined termination of transcription. A linearized pDNA template can be transcribed into mRNA in a mixture including recombinant RNA polymerase (T7, T3 or SP6) and nucleoside triphosphates. To obtain capped mRNA by transcription a cap analog like the dinucleotide m⁷G(5′)-ppp-(5′)G may be included in the reaction. If the cap analog is in excess of GTP, transcription initiates with the cap analog rather than GTP, yielding capped mRNA. Alternatively, the cap may be added enzymatically post transcription. A poly(A) tail may also be added post transcription if it is not provided by the pDNA template. Following transcription, the pDNA template as well as contaminating bacterial DNA is digested by DNase. The resultant mRNA transcript can be purified by a combination of precipitation and extraction steps.

In order to be translated and elicit an antigen-specific immune response, an mRNA-vaccine has to reach the cytosol of target cells. However, as opposed to DNA vaccines, RNA vaccines only have to cross the plasma membrane, but not the nuclear envelope which may improve the probability of successful in vivo transfection. While locally administered naked mRNA can be taken up by cells, the efficacy of mRNA vaccines may benefit significantly from complexing agents which protect RNA from degradation. Complexing agents can be tailored to the specific route of delivery. Complexation may also enhance uptake by cells and/or improve delivery to the translation machinery in the cytoplasm. Thus, in some implementations, mRNA for use in an immunogen composition can be complexed with either lipids or polymers.

In some implementations, the vector is a delivery vehicle comprised of lipid-based compositions, including lipid nanoparticle compositions include but are not limited those described in U.S.

Pat. Publication No. 20200206362, filed as U.S. Pat. Application Serial No. 16/599661 on Oct. 11, 2019 and U.S. Pat. No. 10,799,463, the contents of which are incorporated herein by reference.

In some implementations, the recombinant vector is a self-amplifying RNA (saRNA also called “replicon RNA”). A saRNA can be engineered and derived from genomes of positive-strand, non-segmented RNA viruses such as alphaviruses or flaviviruses. In certain implementations, the saRNA is derived from an alphavirus. The alphaviral genome is divided into two ORFs: the first ORF encodes proteins for the RNA dependent RNA polymerase (replicase), and the second ORF encodes structural proteins. In saRNA vaccine constructs, the ORF encoding viral structural proteins is replaced with any antigen of choice, while the viral replicase remains an integral part of the vaccine and drives intracellular amplification of the RNA after immunization. Therefore, in some implementations, the recombinant vector can include a saRNA vaccine construct where the ORF encoding viral structural proteins have been replaced with two or more selected optimal Coronavirus CTL epitopes.

As an alternative to direct injection of mRNA, an immune response may also be induced by vaccination with APCs transfected with mRNA ex vivo where the APCs (e.g., dendritic cells or DCs) are infused into the subject in need thereof. Transfection of DCs with mRNA encoding two or more optimal Coronavirus CTL epitopes can be accomplished with the use of a cationic lipid, i.e., DOTAP, or electroporation.

Typically, approaches for DC-based vaccination are mainly based on antigen loading on in vitro-generated DCs from monocytes or CD34⁺ cells, activating them with different TLR ligands, cytokine combinations, and injecting them back to a subject in need thereof. DCs can be loaded through incubation with peptides (such as peptide-based vaccine compositions described below), proteins, RNA, or autologous/allogeneic tumor cells. Peptides can loaded directly on the MHC molecules on the surface of the DCs. In addition to RNA electroporation, antigens can be loaded into DCs using bacterial or viral vector transduction. Peptides or proteins can be loaded into DCs and provided one or more maturation stimuli such as proinflammaroty cytokines, CD40L and/or TLR agonists.

In some implementations, bacterial or viral vectors can be used to target DCs with antigens. Exemplary vectors used to target DCs can include, but are not limited to, vectors derived from bacteria such as BCG, Listeria monocytogenes, Salmonella, and Shigella, and viruses including Canarypox virus, Newcastle disease virus, vaccinia virus, Sindbis virus, yellow fever virus, human papillomavirus, adenovirus, adeno-associated virus, and lentiviruses.

In certain implementations, the number of antigen loaded DCs administered to a subject can range from about 0.3 × 10⁶ cells to about 200 × 10⁶ cells per administration. A typical DC vaccination schedule can range from once every 2 weeks vs 3-4 doses or even up to 10 doses given every 3-4 weeks). The route of antigen loaded DC administration to a subject in need thereof can include injection, for example, subcutaneous, intradermal, intranodal, intravenous, or even intratumoral injection. In some implementations, administration strategies include administration of DC vaccines via more than one route, i.e., intradermally plus intravenously to induce a systemic response, and/or administration directly into the lymph nodes (intranodally). In some implementations, a T cell immunogen composition can include a peptide-based vaccine. For example, two or more selected optimal Coronavirus CTL epitope recombinant peptides for vaccination can be produced by expressing the immunogenic peptides in a heterologous expression system, e.g., a yeast expression system. Once purified, recombinant immunogenic peptides are typically administered to a subject with an adjuvant to boost the immune response. Delivery systems used for peptide vaccine use are typically able to protect protease-sensitive epitopes from degradation, and also allow for co-deliver of additional vaccine components such as an adjuvant. Exemplary peptide vaccine delivery systems can include, but are not limited to polymers, lipids (including liposomes, exosomes), inorganic particles, microparticles, nanoparticles, and carbon nanotubes.

As described in more detail below, the T cell immunogen composition can be used to form a therapeutic composition, such as a vaccine or pharmaceutical composition. While it is possible that a vaccine can comprise the T cell immunogen composition in a pure or substantially pure form, it will be appreciated that the vaccine can additionally or optionally include the T cell immunogen composition and a pharmaceutically acceptable carrier or other therapeutic agent. For example, the pharmaceutically acceptable carrier can include a physiologically acceptable diluent, such as sterile water or sterile isotonic saline. As used herein, the term “pharmaceutically acceptable carrier” can refer to any and all solvents, dispersion media, coatings, antibacterial and antifungal agents, isotonic and absorption delaying agents, and the like.

Additional components that may be present with the T cell immunogen composition can include adjuvants, preservatives, chemical stabilizers, and/or other proteins. It will be appreciated that the T cell immunogen composition can be conjugated with one or more lipoproteins, administered in liposomal form, or with an adjuvant. For example, to be efficient, vaccines can include a strong adjuvant supplying a signal for the initiation and support of the adaptive immune response in addition to an appropriate antigen, e.g., two or more selected optimal Coronavirus CTL epitopes.

Typically, stabilizers, adjuvants, and preservatives are optimized to determine the best formulation for efficacy in a subject. Exemplary preservatives can include, but are not limited to, chiorobutanol, potassium sorbate, sorbic acid, sulfur dioxide, propyl gallate, the parabens, ethyl vanillin, glycerin, phenol, and parachiorophenol. Suitable stabilizing ingredients can include, for example, casamino acids, sucrose, gelatin, phenol red, N-Z amine, monopotassium diphosphate, lactose, lactalbumin hydrolysate, and dried milk. Other examples of pharmaceutically acceptable carriers are known in the art and described below.

A T cell immunogen composition described herein administered to a subject as an COVID vaccine can be used either prophylactically or therapeutically.

When provided prophylactically, the vaccine can be provided in advance of any evidence of an active COVID infection and thereby attenuate or prevent COVID infection. For example, a human subject at high risk for COVID infection can be prophylactically treated with a vaccine comprising the T cell immunogen composition and a pharmaceutically acceptable carrier. When provided therapeutically, the vaccine can be used to enhance a subject’s own immune response to the antigens present as a result of COVID infection. Thus, in some implementations, a therapeutically and/or prophylactically effective amount of T cell immunogen composition described herein is an amount that elicits an immune response to two or more optimal Coronavirus CTL epitopes and thereby prevents or inhibits COVID infection in the subject. Method of preventing Coronavirus infection in the subject, or reducing the severity thereof, include treatment of subjects exposed or infected with the P.1 Brazil SARS-CoV-2 variant, B.1.351 South African SARS-CoV-2 variant or B.1.17 United Kingdom SARS-CoV-2 variant. Inhibiting a viral infection can refer to inhibiting the onset of a viral infection, inhibiting an increase in an existing viral infection, or reducing the severity of the viral infection. In this regard, one of ordinary skill in the art will appreciate that while complete inhibition of the onset of a viral infection is desirable, any degree of inhibition of the onset of a viral infection is beneficial. Likewise, one of ordinary skill in the art will appreciate that while elimination of viral infection is desirable, any degree of inhibition of an increase in an existing viral infection or any degree of a reduction of a viral infection is beneficial.

Inhibition of a viral infection can be assayed by methods known in the art, such as by assessing viral load. Viral loads can be measured by methods known in the art, such as by using PCR to detect the presence of viral nucleic acids or antibody-based assays to detect the presence of viral protein in a sample (e.g., blood) from a subject. Alternatively, the number of CD4+ T cells in a viral-infected subject can be measured. A treatment that inhibits an initial or further decrease in CD4+ T cells in a viral-infected subject, or that results in an increase in the number of CD4+ T cells in a viral-infected subject, for example, may be considered an efficacious or therapeutic treatment.

Optimal dosages to be administered may be readily determined by those skilled in the art, and will vary with the particular compound used, the strength of the preparation, the mode of administration, and the advancement of the disease condition. In addition, factors associated with the particular patient being treated, including patient age, weight, diet and time of administration, will result in the need to adjust dosages.

In some implementations, a pharmaceutical composition administered to a subject includes a therapeutically effective amount of the T cell immunogen composition and another therapeutic agent useful in the treatment of COVID infection, such as a component used for highly active antiretroviral therapy (HAART) or immunotoxins.

As noted above, compositions described herein may be combined with one or more additional therapeutic agents useful in the treatment of COVID infection. It will be understood that the scope of combinations of the compounds of this invention with COVID antivirals, immunomodulators, anti-infectives or vaccines is not limited to the following list, and includes in principle any combination with any pharmaceutical composition useful for the treatment of AIDS. The COVID antivirals and other agents will typically be employed in these combinations in their conventional dosage ranges and regimens as reported in the art.

Examples of antiviral agents include (but not restricted) ANTIVIRALS Manufacturer (Tradename and/or Drug Name Location) Indication (Activity): antibody cocktail Casirivimab and Imdevimab Regeneron COVID infection; abacavir GlaxoSmithKline HIV infection, AIDS, ARC GW 1592 (ZIAGEN) (nRTI); 1592U89 abacavir+GlaxoSmithKline HIV infection, AIDS, ARC (nnRTI); lamivudine+(TRIZIVIR) zidovudine acemannan Carrington Labs ARC (Irving, Tex.) ACH 126443 Achillion Pharm. HIV infections, AIDS, ARC (nucleoside reverse transcriptase inhibitor); acyclovir Burroughs Wellcome HIV infection, AIDS, ARC, in combination with AZT AD-439 Tanox Biosystems HIV infection, AIDS, ARC AD-519 Tanox Biosystems HIV infection, AIDS, ARC adefovir dipivoxil Gilead HIV infection, AIDS, ARC GS 840 (RTI); AL-721 Ethigen ARC, PGL, HIV positive, (Los Angeles, Calif.), AIDS alpha interferon GlaxoSmithKline Kaposi’s sarcoma, HIV, in combination w/Retrovir AMD3100 AnorMed HIV infection, AIDS, ARC (CXCR4 antagonist); amprenavir GlaxoSmithKline HIV infection, AIDS, 141 W94 (AGENERASE) ARC (PI); GW 141 VX478 (Vertex) ansamycin Adria Laboratories ARC LM 427 (Dublin, Ohio) Erbamont (Stamford, Conn.) antibody which neutralizes; Advanced Biotherapy AIDS, ARC pH labile alpha aberrant Concepts (Rockville, Interferon Md.) AR177 Aronex Pharm HIV infection, AIDS, ARC atazanavir (BMS 232632) Bristol-Myers-Squibb HIV infection, AIDS, ARC (ZRIVADA) (PI); beta-fluoro-ddA Nat′l Cancer Institute AIDS-associated diseases BMS-232623 Bristol-Myers Squibb/HIV infection, AIDS, (CGP-73547) Novartis ARC (PI); BMS-234475 Bristol-Myers Squibb/HIV infection, AIDS, (CGP-61755) Novartis ARC (PI); capravirine Pfizer HIV infection, AIDS, (AG-1549, S-1153) ARC (nnRTI); CI-1012 Warner-Lambert HIV-1 infection cidofovir Gilead Science CMV retinitis, herpes, papillomavirus curdlan sulfate AJI Pharma USA HIV infection cytomegalovirus immune MedImmune CMV retinitis globin cytovene Syntex sight threatening CMV ganciclovir peripheral CMV retinitis delavirdine Pharmacia-Upjohn HIV infection, AIDS, (RESCRIPTOR) ARC (nnRTI); Remdesivir Gilead Science coronavirus infection; hydroxychloroquine (Plaquenil) Sanofi coronavirus infection; chloroquine (Aralen) Rising Pharmaceuticals coronavirus infection; dextran Sulfate Ueno Fine Chem. Ind. AIDS, ARC, HIV Ltd. (Osaka, Japan) positive asymptomatic ddC Hoffman-La Roche HIV infection, AIDS, ARC (zalcitabine, (HIVID) (nRTI); dideoxycytidine ddl Bristol-Myers Squibb HIV infection, AIDS, ARC; Dideoxyinosine (VIDEX) combination with AZT/d4T (nRTI) DPC 681 & DPC 684 DuPont HIV infection, AIDS, ARC (PI) DPC 961 & DPC 083 DuPont HIV infection AIDS, ARC (nnRTRI); emvirine Triangle Pharmaceuticals HIV infection, AIDS, ARC (COACTINON) (non-nucleoside reverse transcriptase inhibitor); EL10 Elan Corp, PLC HIV infection (Gainesville, Ga.) efavirenz DuPont HIV infection, AIDS, (DMP 266) (SUSTIVA) ARC (nnRTI); Merck (STOCRIN) famciclovir Smith Kline herpes zoster, herpes simplex emtricitabine Triangle Pharmaceuticals HIV infection, AIDS, ARC FTC (COVIRACIL) (nRTI); Emory University emvirine Triangle Pharmaceuticals HIV infection, AIDS, ARC (COACTINON) (non-nucleoside reverse transcriptase inhibitor); HBY097 Hoechst Marion Roussel HIV infection, AIDS, ARC (nnRTI); hypericin VIMRx Pharm. HIV infection, AIDS, ARC recombinant human; Triton Biosciences AIDS, Kaposi’s sarcoma, interferon beta (Almeda, Calif.); ARC interferon alfa-n3 Interferon Sciences ARC, AIDS indinavir; Merck (CRIXIVAN) HIV infection, AIDS, ARC, asymptomatic HIV positive, also in combination with AZT/ddI/ddC (PI); ISIS 2922 ISIS Pharmaceuticals CMV retinitis JE2147/AG1776; Agouron HIV infection, AIDS, ARC (PI); KNI-272 Nat′l Cancer Institute HIV-assoc diseases lamivudine; 3TC Glaxo Wellcome HIV infection, AIDS, (EPIVIR) ARC; also with AZT (nRTI); lobucavir Bristol-Myers Squibb CMV infection; lopinavir (ABT-378) Abbott HIV infection, AIDS, ARC (PI); lopinavir+ritonavir Abbott (KALETRA) HIV infection, AIDS, ARC (ABT-378/r) (PI); mozenavir AVID (Camden, N.J.) HIV infection, AIDS, ARC (DMP-450) (PI); nelfinavir Agouron HIV infection, AIDS, (VIRACEPT) ARC (PI); nevirapine Boeheringer HIV infection, AIDS, Ingleheim ARC (nnRTI); (VIRAMUNE) novapren Novaferon Labs, Inc. HIV inhibitor (Akron, Ohio); pentafusaide Trimeris HIV infection, AIDS, ARC T-20 (fusion inhibitor); peptide T Peninsula Labs AIDS octapeptide (Belmont, Calif.) sequence PRO 542 Progenics HIV infection, AIDS, ARC (attachment inhibitor); PRO 140 Progenics HIV infection, AIDS, ARC (CCR5 co-receptor inhibitor); trisodium Astra Pharm. Products, CMV retinitis, HIV infection, phosphonoformate Inc other CMV infections; PNU-140690 Pharmacia Upjohn HIV infection, AIDS, ARC (PI); probucol Vyrex HIV infection, AIDS; RBC-CD4Sheffield Med. Tech HIV infection, AIDS, (Houston Tex.) ARC; ritonavir Abbott HIV infection, AIDS, (ABT-538) (RITONAVIR) ARC (PI); saquinavir Hoffmann-LaRoche HIV infection, AIDS, (FORTOVASE) ARC (PI); stavudine d4T Bristol-Myers Squibb HIV infection, AIDS, ARC didehydrodeoxy-(ZERIT.) (nRTI); thymidine T-1249 Trimeris HIV infection, AIDS, ARC (fusion inhibitor); TAK-779 Takeda HIV infection, AIDS, ARC (injectable CCR5 receptor antagonist); tenofovir Gilead (VIREAD) HIV infection, AIDS, ARC (nRTI); tipranavir (PNU-140690) Boehringer Ingelheim HIV infection, AIDS, ARC (PI); TMC-120 & TMC-125 Tibotec HIV infections, AIDS, ARC (nnRTI); TMC-126 Tibotec HIV infection, AIDS, ARC (PI); valaciclovir GlaxoSmithKline genital HSV & CMV infections virazole Viratek/ICN (Costa asymptomatic HIV positive, ribavirin Mesa, Calif.) LAS, ARC; zidovudine; AZT GlaxoSmithKline HIV infection, AIDS, ARC, (RETROVIR) Kaposi’s sarcoma in combination with other therapies (nRTI); [PI=protease inhibitor nnRTI=non-nucleoside reverse transcriptase inhibitor NRTI=nucleoside reverse transcriptase inhibitor].

The additional therapeutic agent may be used individually, sequentially, or in combination with one or more other such therapeutic agents described herein (e.g., Coronavirus antivirals, an COVID protein derived from the subject). Administration to a subject may be by the same or different route of administration or together in the same pharmaceutical formulation.

According to this implementation, a T cell immunogen composition described herein may be coadministered with any antiviral regimen or component thereof. For subjects with low to non-measurable levels of plasma COVID RNA over prolonged periods may require less aggressive treatment. For treatment-naive subject who are treated with initial treatment regimen, different combinations (or cocktails) of antiviral drugs can be used.

Thus, in some implementations, a pharmaceutical composition comprising a T cell immunogen composition may be coadministered to the subject with a “cocktail” of COVID antivirals. For example, a pharmaceutical composition including the T cell immunogen composition and COVID antivirals.

Coadministration in the context of this invention is defined to mean the administration of more than one therapeutic agent in the course of a coordinated treatment to achieve an improved clinical outcome. Such coadministration may also be coextensive, that is, occurring during overlapping periods of time.

Pharmaceutical compositions described herein can be formulated by standard techniques using one or more physiologically acceptable carriers or excipients. Suitable pharmaceutical carriers are described herein and in “Remington’s Pharmaceutical Sciences” by E. W. Martin. The small molecule compounds of the present invention and their physiologically acceptable salts and solvates can be formulated for administration by any suitable route, including via inhalation, topically, nasally, orally, parenterally, or rectally. Thus, the administration of the pharmaceutical composition may be made by intradermal, subdermal, intravenous, intramuscular, intranasal, intracerebral, intratracheal, intraarterial, intraperitoneal, intravesical, intrapleural, intracoronary or intratumoral injection, with a syringe or other devices. Transdermal administration is also contemplated, as are inhalation or aerosol administration. Tablets and capsules can be administered orally, rectally or vaginally.

For oral administration, a pharmaceutical composition or a medicament can take the form of, for example, a tablets or a capsule prepared by conventional means with a pharmaceutically acceptable excipient. Preferred are tablets and gelatin capsules comprising the active ingredient, i.e., a small molecule compound of the present invention, together with (a) diluents or fillers, e.g., lactose, dextrose, sucrose, mannitol, sorbitol, cellulose (e.g., ethyl cellulose, microcrystalline cellulose), glycine, pectin, polyacrylates and/or calcium hydrogen phosphate, calcium sulfate; (b) lubricants, e.g., silica, talcum, stearic acid, its magnesium or calcium salt, metallic stearates, colloidal silicon dioxide, hydrogenated vegetable oil, corn starch, sodium benzoate, sodium acetate and/or polyethyleneglycol; for tablets also (c) binders, e.g., magnesium aluminum silicate, starch paste, gelatin, tragacanth, methylcellulose, sodium carboxymethylcellulose, polyvinylpyrrolidone and/or hydroxypropyl methylcellulose; if desired (d) disintegrants, e.g., starches (e.g., potato starch or sodium starch), glycolate, agar, alginic acid or its sodium salt, or effervescent mixtures; (e) wetting agents, e.g., sodium lauryl sulphate, and/or (f) absorbents, colorants, flavors and sweeteners.

Tablets may be either film coated or enteric coated according to methods known in the art. Liquid preparations for oral administration can take the form of, for example, solutions, syrups, or suspensions, or they can be presented as a dry product for constitution with water or other suitable vehicle before use. Such liquid preparations can be prepared by conventional means with pharmaceutically acceptable additives, for example, suspending agents, for example, sorbitol syrup, cellulose derivatives, or hydrogenated edible fats; emulsifying agents, for example, lecithin or acacia; non-aqueous vehicles, for example, almond oil, oily esters, ethyl alcohol, or fractionated vegetable oils; and preservatives, for example, methyl or propyl-p-hydroxybenzoates or sorbic acid. The preparations can also contain buffer salts, flavoring, coloring, and/or sweetening agents as appropriate. If desired, preparations for oral administration can be suitably formulated to give controlled release of the active compound. Pharmaceutical compositions described herein can be formulated for parenteral administration by injection, for example by bolus injection or continuous infusion. Formulations for injection can be presented in unit dosage form, for example, in ampoules or in multi-dose containers, with an added preservative. Injectable compositions are preferably aqueous isotonic solutions or suspensions, and suppositories are preferably prepared from fatty emulsions or suspensions. The compositions may be sterilized and/or contain adjuvants, such as preserving, stabilizing, wetting or emulsifying agents, solution promoters, salts for regulating the osmotic pressure and/or buffers. Alternatively, the active ingredient can be in powder form for constitution with a suitable vehicle, for example, sterile pyrogen-free water, before use. In addition, they may also contain other therapeutically valuable substances. The compositions are prepared according to conventional mixing, granulating or coating methods, respectively, and contain about 0.1 to 75%, preferably about 1 to 50%, of the active ingredient.

For administration by inhalation, the compounds may be conveniently delivered in the form of an aerosol spray presentation from pressurized packs or a nebulizer, with the use of a suitable propellant, for example, dichlorodifluoromethane, trichlorofluoromethane, dichlorotetrafluoroethane, carbon dioxide, or other suitable gas. In the case of a pressurized aerosol, the dosage unit can be determined by providing a valve to deliver a metered amount. Capsules and cartridges of, for example, gelatin for use in an inhaler or insufflator can be formulated containing a powder mix of the compound and a suitable powder base, for example, lactose or starch.

Suitable formulations for transdermal application include an effective amount of a compound of the present invention with carrier. Preferred carriers include absorbable pharmacologically acceptable solvents to assist passage through the skin of the host. For example, transdermal devices are in the form of a bandage comprising a backing member, a reservoir containing the compound optionally with carriers, optionally a rate controlling barrier to deliver the compound to the skin of the host at a controlled and predetermined rate over a prolonged period of time, and means to secure the device to the skin. Matrix transdermal formulations may also be used. Suitable formulations for topical application, e.g., to the skin and eyes, are preferably aqueous solutions, ointments, creams or gels well-known in the art. Such may contain solubilizers, stabilizers, tonicity enhancing agents, buffers and preservatives.

A pharmaceutical composition for use in a method described herein can also be formulated in rectal compositions, for example, suppositories or retention enemas, for example, containing conventional suppository bases, for example, cocoa butter or other glycerides.

Furthermore, the pharmaceutical compositions can be formulated as a depot preparation. Such long-acting formulations can be administered by implantation (for example, subcutaneously or intramuscularly) or by intramuscular injection. Thus, for example, the compounds can be formulated with suitable polymeric or hydrophobic materials (for example as an emulsion in an acceptable oil) or ion exchange resins, or as sparingly soluble derivatives, for example, as a sparingly soluble salt.

The compositions can, if desired, be presented in a pack or dispenser device that can contain one or more unit dosage forms containing the active ingredient. The pack can, for example, comprise metal or plastic foil, for example, a blister pack. The pack or dispenser device can be accompanied by instructions for administration.

In one implementation, a pharmaceutical composition is administered to a subject, preferably a human, at a therapeutically effective dose to prevent, treat, or control a condition or disease as described herein, such as COVID.

The dosage of pharmaceutical compositions administered is dependent on the species of warm-blooded animal (mammal), the body weight, age, individual condition, surface area of the area to be treated and on the form of administration. The size of the dose also will be determined by the existence, nature, and extent of any adverse effects that accompany the administration of a particular small molecule compound in a particular subject. Typically, a dosage of the active compounds of the present invention is a dosage that is sufficient to achieve the desired effect. Optimal dosing schedules can be calculated from measurements of compound accumulation in the body of a subject. In general, dosage may be given once or more daily, weekly, or monthly. Persons of ordinary skill in the art can easily determine optimum dosages, dosing methodologies and repetition rates.

In another implementation, a pharmaceutical composition including a T cell immunogen composition described herein is administered in a daily dose in the range from about 0.1 mg per kg of subject weight (0.1 mg/kg) to about 1 g/kg for multiple days. In another implementation, the daily dose is a dose in the range of about 5 mg/kg to about 500 mg/kg. In yet another implementation, the daily dose is about 10 mg/kg to about 250 mg/kg. In yet another implementation, the daily dose is about 25 mg/kg to about 150 mg/kg. A preferred dose is about 10 mg/kg. The daily dose can be administered once per day or divided into subdoses and administered in multiple doses, e.g., twice, three times, or four times per day.

To achieve the desired therapeutic effect, compositions described herein may be administered for multiple days at the therapeutically effective daily dose. Thus, therapeutically effective administration of a pharmaceutical composition for use as an COVID vaccine described herein in a subject requires periodic (e.g., daily) administration that continues for a period ranging from three days to two weeks or longer. Typically, a pharmaceutical composition will be administered for at least three consecutive days, often for at least five consecutive days, more often for at least ten, and sometimes for 20, 30, 40 or more consecutive days. While consecutive daily doses are a preferred route to achieve a therapeutically effective dose, a therapeutically beneficial effect can be achieved even if the pharmaceutical compositions are not administered daily, so long as the administration is repeated frequently enough to maintain a therapeutically effective concentration of the T cell immunogen composition in the subject. For example, one can administer a pharmaceutical composition every other day, every third day, or, if higher dose ranges are employed and tolerated by the subject, once a week. A preferred dosing schedule, for example, can include administering daily for a week, one week off and repeating this cycle dosing schedule for 3-4 cycles.

Optimum dosages, toxicity, and therapeutic efficacy of a pharmaceutical composition described herein may vary depending on the relative potency of individual compounds and can be determined by standard pharmaceutical procedures in cell cultures or experimental animals, for example, by determining the LD₅₀ (the dose lethal to 50% of the population) and the ED₅₀ (the dose therapeutically effective in 50% of the population). The dose ratio between toxic and therapeutic effects is the therapeutic index and can be expressed as the ratio, LD₅₀/ED₅₀. T cell immunogen compositions that exhibit large therapeutic indices are preferred. While compositions that exhibit toxic side effects can be used, care should be taken to design a delivery system that targets such compounds to the Coronavirus infected cells to minimize potential damage to normal cells and, thereby, reduce side effects.

The data obtained from, for example, cell culture assays and animal studies can be used to formulate a dosage range for use in humans. The dosage of the T cell immunogens in a pharmaceutical composition described herein preferably within a range of circulating concentrations that include the ED₅₀ with little or no toxicity. The dosage can vary within this range depending upon the dosage form employed and the route of administration. For any compositions used in the methods of the invention, the therapeutically effective dose can be estimated initially from cell culture assays. A dose can be formulated in animal models to achieve a circulating plasma concentration range that includes the IC₅₀ (the concentration of the test compound that achieves a half-maximal inhibition of symptoms) as determined in cell culture. Such information can be used to more accurately determine useful doses in humans. Levels in plasma can be measured, for example, by high performance liquid chromatography (HPLC).

Following successful treatment, it may be desirable to have the subject undergo maintenance therapy to prevent the recurrence of the condition or disease treated.

As can be appreciated from the disclosure above, the present invention has a wide variety of applications. The invention is further illustrated by the following examples, which are only illustrative and are not intended to limit the definition and scope of the invention in any way.

### Example 1

### Materials and Methods of the Structure-Based Network Analysis

This approach consists of protein network construction and protein network analysis. For network construction, two approaches were used to infer interactions between individual atoms of amino acid residues: an energetic network and a centroid network. In the energetic network, non-covalent interactions, which include van der Waals interactions, hydrogen bonds, water-bridged bonds, salt bridges, disulfide bonds, pi-pi interactions, pi-cation interactions and metal coordinated bonds, were calculated between pairs of residues based on energy potentials and appropriate angle and distance thresholds using the atomic coordinates found in the Protein Data Bank file (PDB, https://www.rcsb.org/). Protein networks were then constructed by defining each individual amino acid residue within the protein structure as a node and defining weighted edges as the sum of all intermolecular bond energies between residues. Energies for each bond type were defined using previously established values in kJ/mol. For the centroid network, the side chain center of mass for each amino acid residue and defined bonds based on a distance threshold cutoff between centroids of 8.5 angstroms. The purpose of including the centroid network was to account for the contribution of hydrophobic packing to protein folding. Centroid protein networks were then constructed by defining each amino acid residue as a node and defining edges as binary interactions that meet the defined 8.5 angstrom threshold for centroid-to-centroid distance. Edges to immediately neighboring amino acids (n-1, n+1) were not included in either approach due to presence of covalent peptide bonds between these residues. All calculations were carried out in Python.

For protein network analysis, a number filters was applied to calculate network parameters. First, in the energetic network, all edges were considered as well as those strictly between terminal atoms, as previously described, in order to focus on residue-specific interactions. Thus, for an edge to be included, one of the two participating atoms needed to be a terminal atom. Edges were then summed over an amino acid residue to transform the edge list from a list of atom-atom interactions to a list of residue-residue interactions. Second, a filter to calculate network parameters on edges that bridge residues from different higher order protein structures was applied. Higher order protein structures were identified in two ways. First, classical secondary structure was utilized using the publicly available software tool Stride (http://webclu.bio.wzw.tum.de/stride/). Second, network-defined higher order structures were inferred based on a random walk approach whereby tightly connected communities are identified and distinguished (Walktrap, http://igraph.org/r/doc/cluster_walktrap.html). For higher order structure filters, no edges were considered between residues within the same structural motif. Together, these filters were used to calculate three network parameters prior to summation of the final network score. The network parameters are as follows: 1. Second Order Intermodular Degree: the number of second order interactions (two degrees of separation) between residues from different higher order structures, as an average of classical secondary structure and Walktrap definitions.

$$\begin{matrix}S e c o n d o r d e r i n t e r m o d u l a r d e g r e e \left(S D\right) \\ = \frac{{\left({\sum }_{i = 1}^{n} {k}_{i} + {\sum }_{i = 1}^{n} k {s}_{i}\right)}_{e n e r g e t i c} + {\left({\sum }_{i = 1}^{n} {k}_{i} + {\sum }_{i = 1}^{n} {w}_{i}\right)}_{c e n t r o i d}}{4}\end{matrix}$$

where a node has n neighbors in different modules and ki and ksi are the degrees (number of edges) of those neighbors i for the regular energetic network and the terminal atom filtered energetic network, respectively, with higher order structures defined by secondary structure. These values are summed for neighbors 1 through n. If multimeric protein structure data were available, this metric was considered only for the multimer prior to normalization. These calculations were then calculated for the centroid network, where modules defined by both secondary structure (ki) and Walktrap (wi) were used. Each individual value (ki, ksi, wi) was standard normalized before summing. The final SD value was then obtained for each amino acid in the network as an average of the 4 described calculations. The purpose of taking an average of 4 different estimates of second order intermodular degree was to capture the unique contributions of the energetic network, the terminal atom filter, the coarse-grained centroid network and the Walktrap higher order structure definition.

2. Node Edge Betweenness: the summed frequency that a node’s edges were utilized as a shortest path between all pairs of nodes in the network, weighted by edge weight

$$E d g e i n t e r m o d u l a r b e t w e e n n e s s \left(E B\right) = {\sum }_{j = 1 , k = 1 , j \neq k}^{j = n , k = n , j \neq k} {e}_{j k}$$

where ejk = 1 if edge ejk is used in the shortest path between nodes j and k, otherwise ejk = 0. Only edges between nodes of different higher order structure were allowed, and here the structures were defined by secondary structure. These counts were then summed for all pairs of nodes 1 through n. This edge parameter is then converted into a node parameter:

$$\begin{matrix}N o d e e d g e i n t e r m o d u l a r b e t w e e n n e s s \left(N E B\right) = \\ \frac{{\sum }_{i = 1}^{n} E {B}_{i} + {\sum }_{i = 1}^{n} E B {S}_{i}}{2}\end{matrix}$$

where EB was the edge betweenness for each edge i for a node with n neighbors and EBS was the same metric but for the network filtered on sidechain interactions. These metrics are standard normalized and then averaged. If a multimeric version of the protein exists, then the maximum node edge betweenness is taken between the monomeric and multimeric conformations.

3. Euclidean Distance from Centroid to Ligand: the distance in angstroms of a residue’s centroid to the center of mass of the protein’s ligand. Centroid was defined as the center of mass of a residue’s sidechain, weighted by atomic weight, as described previously:

$$C e n t r o i d \left(C\right) = \frac{{\sum }_{x = 1}^{s} {a}_{x} \left(x , y , z\right)}{s}$$

$$L i g a n d D i s t a n c e \left(L D\right) = \left|C - l i g a n {d}_{c e n t e r o f m a s s}\right|$$

where ax is the atomic weight for atom x in a protein’s sidechain for atoms 1 through s. The (x,y,z) 3-dimensional coordinates were defined in the PDB file. The center of mass of the ligand was calculated using all atoms. The final centroid value was standard normalized and averaged. Final network score was a sum of the aforementioned terms, which had been individually normalized:

$$S D + N E B - L D = f i n a l n e t w o r k s c o r e$$

These values were calculated in R with the assistance of the iGraph package to load networks. PDB Structures: For the validation data set, the following PDB files were used: HSP90 (2CG9, Chains A and B and ATP ligand), Hepatitis C NS5A (3FQM; chains A and B), CCdB toxin (1×75; chains C and D and DNA gyrase ligand), Hemagglutinin (1RVX; chains A, B, C, D, E, F); Gene V Protein (1GVP; chains A, B), Beta-Glucosidase (1GNX; chains A and B), ubiquitin (200B; Chains A and B and Cbl-b ubiquitin ligase ligand), Kanamycin Kinase (1ND4; chains A and B and Kanamycin ligand), DNA binding protein Gal4 (3COQ; chains A and B and DNA ligand), DNA Methylase (1DCT; chain A and DNA ligand), Beta-lactarnase (1BTL; chain A), streptococcal protein G (1FCC; chains A and B and IGG1 Fc protein ligand), T4 lysozyme (2LZM; chain A). For the analysis of the SARS-CoV2 proteome, the following PDB files were utilized: For the analysis of the SARS-CoV-2 proteome, the following PDB files were utilized: NSP3 ADP ribose phosphatase domain (PDB: 6W02), NSP3 papain-like protease (PDB: 6W9C), NSP5 3CL protease (PDB: 6YB7), NSP7 (PDB: 6M7I, Chain C). NSP8 (PDB: 6M7I, Chain B, D), NSP9 (PDB: 6W4B), NSP10 (6W4H, Chain B), NSP12 RNA-dependent RNA polymerase (6M7I, Chain A), NSP15 (PDB: 6W01), NSP16 (PDB: 6W4H, Chain A), Spike closed conformation (PDB: 6VXX), Nucleocapsid RNA-binding domain (PDB: 6VYO), Nucleocapsid dimerization domain (PDB: 6WJI), ORF3a (PDB: 6XDC), ORF7a (PDB: 6W37), Spike open conformation (PDB: 6VYB), and Spike receptor binding domain (PDB: 6M0J). The membrane structure was downloaded from DeepMind and MODELLER was used to create homology models for the envelope protein using SARS-CoV-1 envelope (PDB: 5×29) as a template. Water molecules and solvents were removed from each PDB file prior to analysis.

Calculation of Network Scores for Multimeric Proteins: For multimeric proteins, degree-based network values (second order degree, ligand binding) in the protein’s highest oligomeric state were utilized prior to calculation of a normalized Z-score. For node edge betweenness metrics, the maximum normalized Z-score from monomer, multimeric or inter-multimeric conformations was incorporated into the final network score calculation. Mutated residues engineered to stabilize protein conformations (e.g. 5HGL, Cys14 and Cys45, engineered disulfide bond) were excluded from the analysis. For analyses with multiple structures utilized to capture different conformational states for the same oligomeric structure (e.g. 5HGL and 5HGN, open and closed conformations), network Z-scores were averaged. All molecular assemblies were generated using the online server PDBePISA (http://www.ebi.ac.uk/pdbe/pisa/).

Correlation of Network Scores with Functional Datasets: Composite network scores were correlated against functional datasets obtained from high and low-throughput mutagenesis studies. For TEM-1 Beta-lactamase, network scores were correlated against functional mutant values obtained from the Ampicillin 2500 ug/mL dataset, which was the maximum concentration utilized in the study. For DNA methylase HaeIII, correlations were made using the dataset after the full 17 rounds of mutagenesis. For NS5A, the dataset for the virus under selection was analyzed with Daclatasvir. For Kanamycin Kinase, the 1:8 Kanamycin dilution dataset was used.

For the remaining proteins, the single supplementary datasets provided were utilized for correlative studies. Each set of functional scores for a given protein was standard normalized by subtracting the mean and dividing by the standard deviation.

Calculation of Shannon Entropy: Multiple sequence alignments were downloaded from PFAM (http://pfam.xfam.org). Using the protein sequence derived from the protein’s PDB structure as a reference in each protein sequence alignment, amino acid frequencies were tabulated at each amino acid position in the corresponding aligned orthologous proteins. Shannon entropy H(p) was calculated based on the following formula: H(p) = - Σa pₐ log₂ (pₐ) where pₐ is the proportion of amino acid a at a given position and qₐ is the background frequency of amino acid a. Residues with uncertain alignment per PFAM were excluded from downstream analyses. The background frequencies used were the frequencies of each amino acid across the entire alignment.

Calculation of Relative Solvent Accessibility: Relative Solvent Accessibility (RSA) values were calculated by using the following formula: RSA = Accessible Solvent Area (ASA) / Maximum ASA, with ASA values calculated using the publicly available software tool Stride (http://webclu.bio.wzw.tum.de/stride/) and utilizing previously reported MaxASA values. Receiver Operator Curves: Receiver Operator Curves (ROC) were plotted and calculated in R using the pROC library to determine the predictive ability of network scores, Shannon entropy and relative solvent accessibility values to determine the top 10% of residues ranked by mutational intolerance. Calculation of CoV Sequence Entropy: Values for CoV sequence entropy were obtained from the NCBI Virus Sequence Database (https://www.ncbi.nlm.nih.gov/labs/virus/vssi/#/).

Calculation of Epitope Network Scores: Network scores from individual amino acid residues within and neighboring a CD8+ T cell epitope were combined and averaged based on their involvement as either an HLA anchor, TCR contact or peptide processing residues. HLA anchor residues were defined based on previous delineations for each HLA allele. Putative TCR. contact residues were considered to be all remaining non-HLA anchor residues, excluding position 1, based on previously reported frequencies of TCR-peptide contacts. Flanking residues were defined as the five residues N-terminal and C-terminal to the epitope (ten in total). These three quantities were then summed to generate an overall composite network score for each CD8+ T cell epitope. The normalized epitope network score was calculated by subtracting the lowest epitope network score from all epitope scores, such that all values were greater than or equal to zero. The normalized network score was utilized when comparing patient responses such that no CTL response would be assigned a negative value.

### Example 2

### Multi-Networked Epitope Vaccine for Universal Coronavirus Protection

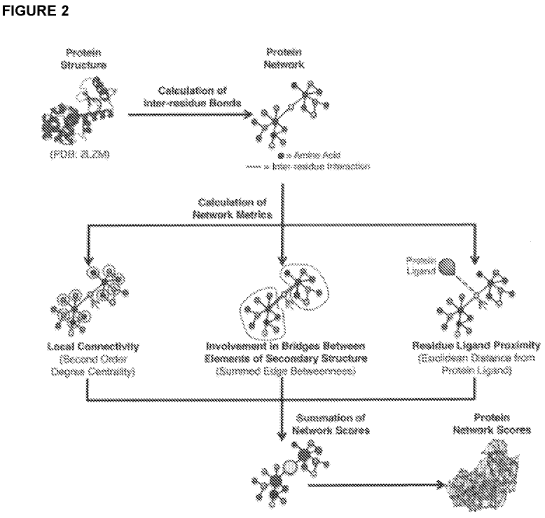

In this Example, a T cell-based immunogen was developed that incorporates mutation resistant epitopes that have been identified through an algorithm known as structure-based network analysis algorithm. The epitopes identified by this analysis are known as networked epitopes. The structure-based network analysis algorithm utilizes protein structure data and network theory metrics to quantify the topological importance of each amino acid residue to a protein’s tertiary and quaternary structure. This is accomplished by using atomic level coordinate data from protein crystal structures to build networks of amino acid residues (nodes) and non-covalent interactions (edges), which included van der Waals interactions, hydrogen bonds, salt bridges, disulfide bonds, pi-pi interactions, pi-cation interactions, metal coordinated bonds and local hydrophobic packing. These inter-residue interactions were calculated between pairs of amino acids using energy potentials and established distance thresholds and summed to generate the protein network. Using this network-based representation, a number of network centrality metrics (measures of relative importance in a given network topology) are calculated, which leads to a quantitative measure of the topological importance of individual amino acid residues through an assessment of a residue’s (i) local connectivity to other residues, (ii) involvement as a bridge between higher order protein elements (secondary structure, tertiary and quaternary structure interfaces) and (iii) proximity to known protein ligands. Integration of these metrics into a single value generates a network score that quantifies the contribution of each amino acid residue to the protein’s topological structure (FIG. 2).

Structure-based network analysis utilizes protein structure data and network theory to quantify the topological importance of each amino acid residue to a protein’s tertiary and quaternary structure. While structural topology has been demonstrated to be a key attribute of residues involved in protein folding, hydrophobic packing and host-pathogen interactions, the network approach was specifically optimized to model the relationship between residue topology and mutational tolerance by focusing on interactions made by atoms unique to an amino acid’s identity. This was accomplished by using atomic level coordinate data from the Protein Data Bank (PDB; https://www.rcsb.org/) to build networks of amino acid residues (nodes) and non-covalent interactions (edges), which included van der Waals interactions, hydrogen bonds, salt bridges, disulfide bonds, pi-pi interactions, pi-cation interactions, metal coordinated bonds and local hydrophobic packing. These inter-residue interactions were calculated between pairs of amino acids using energy potentials and established distance thresholds and summed to generate the protein network (FIG. 2). Using this network-based representation, an array of network centrality metrics was calculated (measures of relative importance in a given network topology), which led to a quantitative measure of the topological importance of individual amino acid residues through an assessment of (i) their local connectivity to other residues, (ii) their involvement as bridges between higher order protein elements (secondary structure, tertiary and quaternary structure interfaces) and (iii) their proximity to known protein ligands (FIG. 2). Integration of these metrics into a single value generated a network score that quantified the relative contribution of each amino acid residue to the protein’s topological structure.

### Example 3

### Structure-Based Network Analysis of SARS-CoV-2 Identifies Residues Highly Conserved Across Circulating SARS-CoV-2 Variants and the Sarbecovirus Subgenus

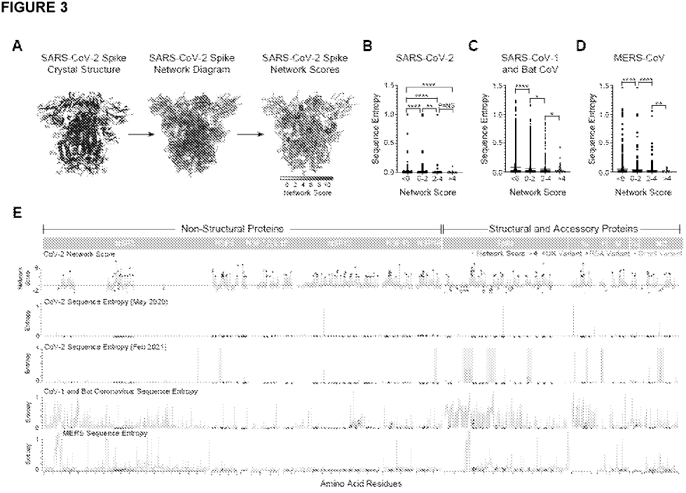

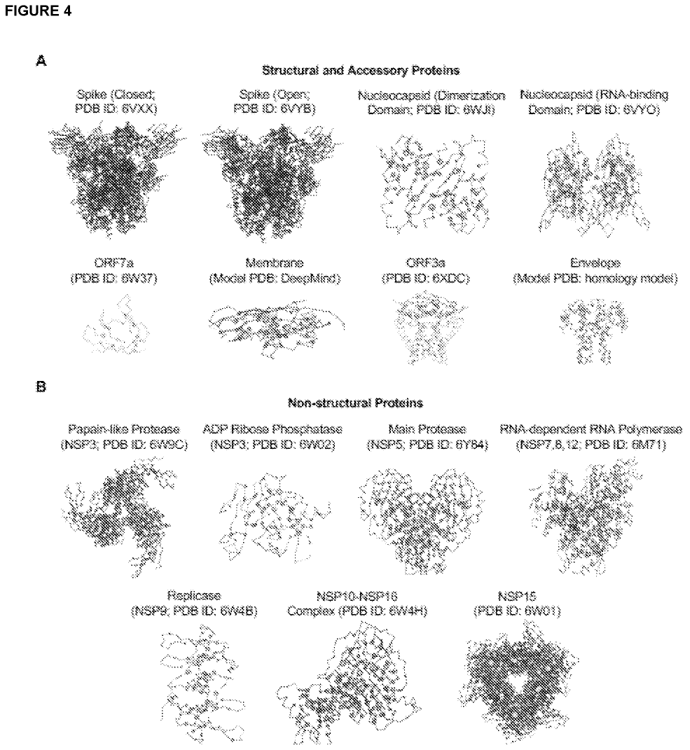

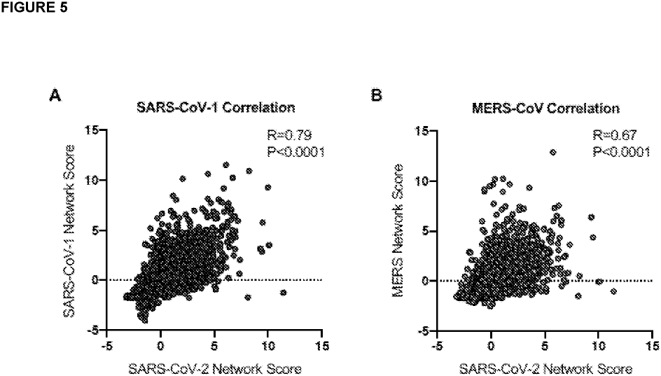

To identify mutation-resistant regions in the SARS-CoV-2 proteome, structure-based network analysis was applied to define topologically important, structurally constrained regions in viral proteins, which were previously utilized for HIV (Gaiha et al., 2019). Based on the availability of high-quality structural data, amino acid network scores were calculated for monomeric and trimeric Spike protein conformations (FIG. 3A) and 14 additional viral proteins, which made up ~44% of the viral proteome (FIG. 4). Residue network scores were binned (<0, 0-2, 2-4, >4) and compared with viral sequence entropy values from SARS-CoV-2, the Sarbecovirus subgenus (SARS-CoV-1/Bat CoV) and MERS-CoV sequences. This revealed a strong inverse relationship between network measures of topological importance in SARS-CoV-2 and mutational frequencies across SARS-CoV-2 (FIG. 3B), sarbecoviruses and MERS-CoV (FIGS. 3C and 3D). Network scores calculated using structural data for SARS-CoV-1 and MERS-CoV were also highly correlated with network scores obtained for SARS-CoV-2 (R = 0.78, R = 0.67, respectively), indicating that highly networked SARS-CoV-2 residues may be structurally conserved across lineage B and C betacoronaviruses (FIG. 5). Moreover, alignment of SARS-CoV-2 residue network scores with viral sequence entropy values for SARS-CoV-2, sarbecoviruses and MERS-CoV revealed numerous linear regions across the SARS-CoV-2 proteome in which highly networked (scores >4), highly conserved CD8⁺ T cell epitopes could putatively be identified (FIG. 3E). The network scores of the mutant residues found in the UK (B.1.1.7), S. African (B.1.351) and Brazilian (P.1) variants were evaluated, and it was determined that the vast majority were poorly networked, with ~89% having negative or undefined values and ~97% having network scores <1 (FIG. 3E, Table 1). This was similar to network scores of Spike escape variants identified by deep mutational scanning (Greaney et al., 2021b) (Table 1). Collectively, these results demonstrate that highly networked amino acid residues, if present within a CD8+ T cell epitope, would have the potential to serve as valuable targets in a broad, mutation-resistant T cell-based vaccine.

TABLE 1

Network Scores of SARS-CoV-2 Residues with Mutations in Naturally Occurring SARS-CoV-2 Variants and In Vitro Studies

| Gene | AA | Network Score | Source |
|---|---|---|---|
| spike | L18F | - | P.1 Brazil variant |
| spike | T20N | - | P.1 Brazil variant |
| spike | P26S | - | P.1 Brazil variant |
| spike | H69 deletion | -1.43669928 | B.1.1.7 UK variant |
| spike | V70 deletion | - | B.1.1.7 UK variant |
| spike | D80A | -0.55792993 | B. 1. 3 5 1 South Africa variant |
| spike | D138Y | -1.19761315 | P.1 Brazil variant |
| spike | Y144 deletion | - | B.1.1.7 UK variant |
| spike | R190S | -0.69165936 | P.1 Brazil variant |
| spike | D215G | -0.67499802 | B. 1. 3 5 1 South Africa variant |
| spike | K417N | -0.86401417 | B. 1. 3 5 1 South Africa variant |
| spike | K417T | -0.86401417 | P.1 Brazil variant |
| spike | E484K | - | B.1.351 South Africa variant, P.1 Brazil variant |
| spike | N501Y | -0.71155894 | B.1.1.7 UK, B.1.351 South Africa, and P.1 Brazil variant |
| spike | A570D | 2.591760471 | B.1.1.7 UK variant |
| spike | D614G | -0.35946146 | D614G variant |
| spike | H655Y | -0.83131904 | P.1 Brazil variant |
| spike | P681H | - | B.1.1.7 UK variant |
| spike | A701V | -1.15312712 | B. 1. 3 5 1 South Africa variant |
| spike | T716I | -0.73665512 | B.1.1.7 UK variant |
| spike | S982A | -0.40343483 | B.1.1.7 UK variant |
| spike | T1027I | -0.1777623 | P.1 Brazil variant |
| spike | D1118H | -0.637657 | B.1.1.7 UK variant |
| nucleocapsid | D3L | - | B.1.1.7 UK variant |
| nucleocapsid | P80R | 0.00090473 | P.1 Brazil variant |
| nucleocapsid | T205I | - | B. 1. 3 5 1 South Africa variant |
| nucleocapsid | S235F | - | B.1.1.7 UK variant |
| envelope | P71L | - | B. 1. 3 5 1 South Africa variant |
| orf3a | G174C | -0.21344791 | P.1 Brazil variant |
| orf1ab | T1001I | - | B.1.1.7 UK variant |
| orf1ab | K1655N | -0.63445263 | B. 1. 3 5 1 South Africa variant |
| orf1ab | I2230T | - | B.1.1.7 UK variant |
| orf1ab | A1708D | 0.653393624 | B.1.1.7 UK variant |
| orf1ab | K1795Q | 0.425004214 | P.1 Brazil variant |
| orf1ab | nuc 11288:9 | - | P.1 Brazil variant |
| orf8 | Q27stop | - | B.1.1.7 UK variant |
| orf8 | R521 | - | B.1.1.7 UK variant |
| orf8 | Y73C | - | B.1.1.7 UK variant |
| orf8 | E92K | - | P.1 Brazil variant |
| spike | N148 | - | Greaney et al., Cell Host and Microbe, Jan. 13, 2021 |
| spike | K150 | - | Greaney et al., Cell Host and Microbe, Jan. 13, 2021 |
| spike | S151 | - | Greaney et al., Cell Host and Microbe, Jan. 13, 2021 |
| spike | R346 | -1.152342 | Greaney et al., Cell Host and Microbe, Jan. 13, 2021 |
| spike | C361 | 0.161711413 | Greaney et al., Cell Host and Microbe, Jan. 13, 2021 |
| spike | V362 | 2.620429496 | Greaney et al., Cell Host and Microbe, Jan. 13, 2021 |
| spike | N370 | -1.28466009 | Greaney et al., Cell Host and Microbe, Jan. 13, 2021 |
| spike | A372 | -1.23763738 | Greaney et al., Cell Host and Microbe, Jan. 13, 2021 |
| spike | T376 | -0.57234679 | Greaney et al., Cell Host and Microbe, Jan. 13, 2021 |
| spike | V382 | 2.715374696 | Greaney et al., Cell Host and Microbe, Jan. 13, 2021 |
| spike | P384 | 1.511814942 | Greaney et al., Cell Host and Microbe, Jan. 13, 2021 |
| spike | R408 | -1.21333506 | Greaney et al., Cell Host and Microbe, Jan. 13, 2021 |
| spike | A411 | 0.408285701 | Greaney et al., Cell Host and Microbe, Jan. 13, 2021 |
| spike | K417 | -0.86401417 | Greaney et al., Cell Host and Microbe, Jan. 13, 2021 |
| spike | K444 | -1.304022 | Greaney et al., Cell Host and Microbe, Jan. 13, 2021 |
| spike | V445 | - | Greaney et al., Cell Host and Microbe, Jan. 13, 2021 |
| spike | G446 | - | Greaney et al., Cell Host and Microbe, Jan. 13, 2021 |
| spike | N450 | -1.25547496 | Greaney et al., Cell Host and Microbe, Jan. 13, 2021 |
| spike | L452 | -0.49721763 | Greaney et al., Cell Host and Microbe, Jan. 13, 2021 |
| spike | P463 | -0.89226659 | Greaney et al., Cell Host and Microbe, Jan. 13, 2021 |
| spike | A475 | - | Greaney et al., Cell Host and Microbe, Jan. 13, 2021 |
| spike | E484 | - | Greaney et al., Cell Host and Microbe, Jan. 13, 2021 |
| spike | G485 | - | Greaney et al., Cell Host and Microbe, Jan. 13, 2021 |
| spike | F490 | -1.061427 | Greaney et al., Cell Host and Microbe, Jan. 13, 2021 |
| spike | Q493 | -1.033094 | Greaney et al., Cell Host and Microbe, Jan. 13, 2021 |
| spike | S494 | -1.22954023 | Greaney et al., Cell Host and Microbe, Jan. 13, 2021 |
| spike | N501 | -0.71155894 | Greaney et al., Cell Host and Microbe, Jan. 13, 2021 |
| spike | V503 | -0.79318694 | Greaney et al., Cell Host and Microbe, Jan. 13, 2021 |

Collectively, these results demonstrate that highly networked amino acid residues, if present within a CD8+ T cell epitope, would have the potential to serve as valuable targets in a broad, mutation-resistant SARS-CoV-2 T cell-based vaccine.

### Example 4

### Mutation of Networked SARS-CoV-2 Spike Residues Impairs Pseudotyped Lentiviral Infectivity

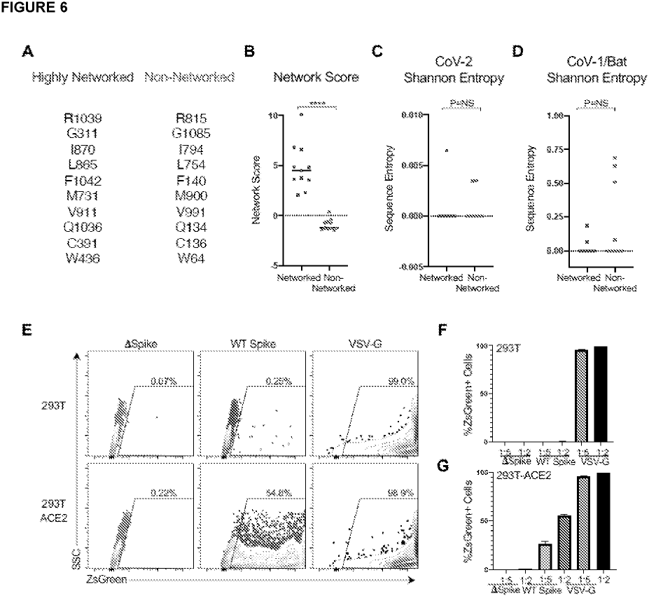

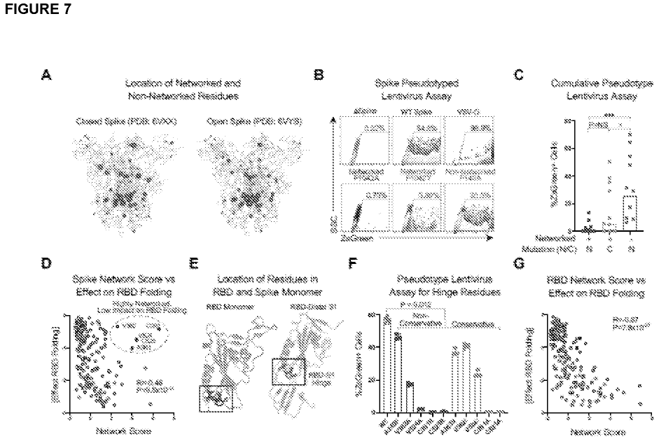

To experimentally evaluate the relationship between SARS-CoV-2 network scores and mutational tolerance, a SARS-CoV-2 Spike pseudotyped lentivirus assay was utilized (Crawford et al., 2020) to engineer nonconservative point mutations for ten pairs of sequence conserved Spike residues which occupied either high (>2; blue) or low (<0.5; red) network score positions (FIG. 7A, FIGS. 6A-D, Table 2). Conservative point mutations for the highly networked Spike residues were also engineered in order to more comprehensively assess their mutational tolerance (Table 1). Pseudotyped lentiviruses with no Spike protein (delta Spike), wild-type (WT) Spike protein or mutant Spike proteins were used to infect parental 293T cells or 293T cells expressing human ACE2 (293T-ACE2), the receptor for viral entry, and the level of infectivity was determined by ZsGreen expression following 3-day incubation. As previously demonstrated (Crawford et al., 2020), no infection of 293T cells was observed by WT Spike pseudotyped lentiviruses but robust infection of 293T-ACE2 cells (FIGS. 6E-G), indicating the clear reliance of pseudotyped Spike-ACE2 interactions. In comparison, vesicular stomatitis virus (VSV)-G envelope pseudotyped lentiviruses, which targets ubiquitous membrane phospholipids, efficiently infected both 293T and 293T-ACE2 cells (FIGS. 6E-G).

Comparative assessment of pseudotyped lentiviruses harboring nonconservative mutations of either highly networked or poorly networked Spike residues revealed highly statistically significant differences in pseudotyped lentiviral infectivity of 293T-ACE2 cells (FIGS. 7B and 7C). Moreover, mutation of highly networked Spike residues with conservative amino acid changes in the same biochemical class (Table 2; FIG. 7) also led to substantial impairment of pseudotyped lentiviral infectivity (FIGS. 7B and 7C). Importantly, highly networked and non-networked Spike residues chosen for mutagenesis had no significant difference in viral sequence entropy across SARS-CoV-2 and the Sarbecovirus subgenus (FIGS. 6C and 6D), indicating that network score provides an additional level of resolution of mutational constraint beyond sequence entropy, consistent with previous observations (Gaiha et al., 2019).

TABLE 2

Engineered Mutations in HDM-SARS2-Spike-delta21

| AA Mutation | Network Score | Entropy (CoV-2) | Entropy (CoV-⅟Bat) | AA Nuc # | Codon | Mutant | Forward Primer (Mutant) | Reverse Primer |
|---|---|---|---|---|---|---|---|---|
| R1039K | 10.09356945 | 0 | 0 | 3117...3119 | AGG | AAG | GCAGTCAAAGaagGTAGATTTCTG | CCAAGCACACATTCAGAC |
| R1039A | 10.09356945 | 0 | 0 | 3117...3119 | AGG | GCG | GCAGTCAAAGgcgGTAGATTTCTGC | CCAAGCACACATTCAGAC |
| R815A | -0.709030275 | 0 | 0 | 2445...2447 | AGG | GCG | ACCTAGTAAGgcgTCATTCATTGAG GATCTTCTGTTTAACAAAG | TTGGAAGGGTCCGGCAGG |
| G311A | 3.661155681 | 0 | 0 | 933...935 | GGC | GCC | GGTGGAGAAAgccATTTATCAGAC | GTGAAGCTCTTAAGGGTG |
| G311V | 3.661155681 | 0 | 0 | 933...935 | GGC | GTC | GGTGGAGAAAgtcATTTATCAGAC | GTGAAGCTCTTAAGGGTG |
| G1085V | -0.75244586 | 0 | 0 | 3255...3257 | GGT | GTC | ATGCCATGATgtcAAGGCGCACTTT CCAAGG | ATAGCGGGCGCCGTCGTA |
| I870L | 3.756766712 | 0 | 0.186051098 | 2610...2612 | ATA | CTA | CGATGAGATGctaGCGCAGTACACG | GTGAGCAGAGGCGGCAGA |
| I870A | 3.756766712 | 0 | 0.186051098 | 2610...2612 | ATA | GCT | CGATGAGATGgctGCGCAGTACACG AGCG | GTGAGCAGAGGCGGCAGA |
| I794A | -1.26346564 | 0.00350491 1 | 0.5085052 | 2382...2384 | ATA | GCT | GACACCACCCgctAAGGACTTCG | TTGTAGATTTGTTTGACCTG |
| L865M | 4.48979055 | 0 | 0 | 2595...2597 | CTC | ATG | GCCGCCTCTGatgACCGATGAGA | AGAACGGTGAGGCCGTTAAA TTTC |
| L865A | 4.48979055 | 0 | 0 | 2595...2597 | CTC | GCT | GCCGCCTCTGgctACCGATGAGATG | AGAACGGTGAGGCCGTTAAA TTTC |
| L754A | -1.197532573 | 0 | 0 | 2262...2264 | CTT | GCT | TAACTTGCTCgctCAGTACGGTTCCT TCTGTAC | CTGCACTCCGTGCTGTCG |
| F1042Y | 4.858035945 | 0 | 0 | 3126...3128 | TTC | TAC | GAGGGTAGATtacTGCGGAAAGG | TTTGACTGCCCAAGCACA |
| F1042A | 4.858035945 | 0 | 0 | 3126...3128 | TTC | GCC | GAGGGTAGATgccTGCGGAAAGG | TTTGACTGCCCAAGCACA |
| F140A | -1.2406754 | 0 | 0.080572811 | 420...422 | TTC | GCC | TAATGATCCCgccCTGGGCGTCT | CAGAATTGAAACTCGCAC |
| M731I | 2.062407959 | 0.00645564 | 0.065211682 | 2193...2195 | ATG | ATC | GCCGGTCTCCatcACCAAGACAT | AGAATCTCAGTGGTGACCG |
| M73 1A | 2.062407959 | 0.00645564 | 0.065211682 | 2193...2195 | ATG | GCG | GCCGGTCTCCgcgACCAAGACATC | AGAATCTCAGTGGTGACCG |
| M900A | 0.405929624 | 0 | 0 | 2700...2702 | ATG | GCG | ACCCTTTGCTgcgCAGATGGCTTAT C | ATCTGCAACGCTGCTCCT |
| V911I | 3.558971164 | 0 | 0 | 2733..2735 | GTC | ATC | CGGGATTGGCatcACGCAGAACG | TTAAATCGATAAGCCATCTG CATAGC |
| V911A | 3.558971164 | 0 | 0 | 2733..2735 | GTC | GCT | CGGGATTGGCgctACGCAGAACG | TTAAATCGATAAGCCATCTG CATAG |
| V991A | -1.436699275 | 0 | 0 | 2973...2975 | GTT | GCT | AGAAGCCGAAgctCAGATTGACC | ACCTTGTCCAACCGTGAC |
| Q1036E | 4.825122097 | 0 | 0 | 3108...3110 | CAG | GAG | TGTGCTTGGGgagTCAAAGAGGG | CATTCAGACATCTTAGTGGC AGC |
| Q1036A | 4.825122097 | 0 | 0 | 3108...3110 | CAG | GCT | TGTGCTTGGGgctTCAAAGAGGGTA G | CATTCAGACATCTTAGTGG |
| Q134A | -1.315359112 | 0.00351397 2 | 0.629091648 | 402...404 | CAA | GCA | GTGCGAGTTTgcaTTCTGTAATG | ACTTTGATGACCACGTTC |
| C391A | 6.583868745 | 0 | 0 | 1173...1175 | TGT | GCT | GAACGATCTCgctTTCACAAACGTT TATGCGG | AGCTTCGTTGGAGACACG |
| C391R | 6.583868745 | 0 | 0 | 1173...1175 | TGT | AGG | GAACGATCTCaggTTCACAAACGTT TATG | AGCTTCGTTGGAGACACG |
| C136R | -1.391725931 | 0 | 0 | 408...410 | TGT | AGG | GTTTCAATTCaggAATGATCCCTTC C | TCGCACACTTTGATGACC |
| W436F | 2.307786446 | 0 | 0 | 1308...1310 | TGG | TTT | TGTCATAGCTtttAATAGCAATAATT TG | CATCCTGTGAAATCGTCC |
| W436A | 2.307786446 | 0 | 0 | 1308...1310 | TGG | GCT | TGTCATAGCTgctAATAGCAATAAT TTGG | CATCCTGTGAAATCGTCC |
| W64A | -0.369087936 | 0.00349100 4 | 0.688997042 | 192...194 | TGG | GCG | CAATGTGACGgcgTTTCATGCCATT C | CTAAAGAAAGGGAGGAAC |
| V3621 | 2.620429497 | 0 | 0.042400539 | 1086...1088 | GTG | ATC | TTCCAATTGTatcGCGGACTACTC | ATTCTCTTTCGGTTCCATG |
| V362A | 2.620429497 | 0 | 0.042400539 | 1086...1088 | GTG | GCG | TTCCAATTGTgcgGCGGACTACT | ATTCTCTTTCGGTTCCATGC |
| V5241 | 4.420173389 | 0 | 0 | 1572...1574 | GTA | ATC | TCCAGCAACGatcTGCGGTCCTA | GCGTGGAGCAATTCGAAAC |
| V524A | 4.420173389 | 0 | 0 | 1572...1574 | GTA | GCA | TCCAGCAACGgcaTGCGGTCCTA | GCGTGGAGCAATTCGAAACT C |
| C525A | 4.44682697 | 0 | 0.081462027 | 1575...1577 | TGC | GCT | AGCAACGGTAgctGGTCCTAAGAAA TCCACAAATC | GGAGCGTGGAGCAATTCG |
| C525R | 4.44682697 | 0 | 0.081462027 | 1575...1577 | TGC | AGG | AGCAACGGTAaggGGTCCTAAGAA ATC | GGAGCGTGGAGCAATTCG |
| A363V | 4.014172971 | 0 | 0.023508256 | 1089...1091 | GCG | GTG | CAATTGTGTGgtgGACTACTCAG | GAAATTCTCTTTCGGTTCCAT G |
| A363F | 4.014172971 | 0 | 0.023508256 | 1089...1091 | GCG | TTT | CAATTGTGTGtttGACTACTCAGTAT TGTATAATAG | GAAATTCTCTTTCGGTTCC |

To more comprehensively assess the mutational tolerance of highly networked residues, a high-throughput mutagenesis dataset was utilized in which every residue within the monomeric receptor binding domain (RBD) of Spike was mutated to all possible amino acid substitutions and assessed for its impact on protein folding stability using yeast surface display (Starr et al., 2020). Correlation of trimeric full Spike residue network scores with average effects of residue mutations on RBD folding stability revealed a significant inverse correlation (R=-0.46, P=9.5×10⁻¹¹) (FIG. 7D). Interestingly, there were five highly networked residues that did not have much impact on RBD monomeric protein folding stability when mutated (V362, A363, C391, V524, C525) (FIG. 7D). The protein structure of the monomeric RBD (PDB ID: 6MOJ) was evaluated, which demonstrated that these residues are not within the RBD core (FIG. 7E) and therefore likely explains why they have little effect on monomeric RBD folding stability (Starr et al., 2020). However, evaluation of the location of these residues in the full Spike structure (PDB ID: 6VXX) was utilized for network score calculations, and revealed that they are located at a critical bridging hinge region between the RBD and distal S1 domain (FIG. 7E) that has been shown to mediate the conformational change between the open and closed states of the viral Spike protein (Gur et al., 2020; Meirson et al., 2020).

Conservative and non-conservative mutations for each of these five Spike residues were engineered (Table 2) and found to have significant effects on pseudotyped lentiviral infectivity, particularly C391, V524 and C525 (FIG. 7F). Network scores for the RBD monomer alone were generated (PDB ID: 6MOJ) and a more robust inverse correlation with protein folding stability was observed (R = -0.67, P=7.9×10⁻²⁷) (FIG. 7G), indicating better agreement between the two methodologies when the same protein domain was used. Collectively, these data demonstrate that structure-based network analysis was not only able to comprehensively identify residues of structural importance in the Spike RBD, but could also delineate key residues not identified by deep mutational scanning, further validating the ability of the approach to define regions of mutational constraint across the SARS-CoV-2 proteome.

### Example 5

### Identification of Highly Networked CD8+ T Cell Epitopes That Stabilize HLA Class I Alleles

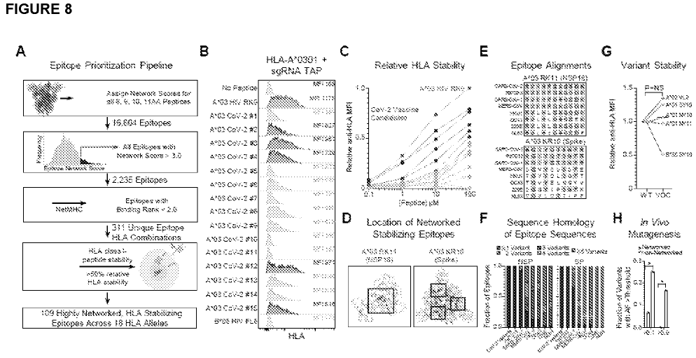

To identify CD8⁺ T cell epitopes within highly networked regions of SARS-CoV-2, a prioritization pipeline that integrated computational epitope prediction with experimental HLA class I stabilization was utilized (FIG. 8A). Epitope network scores were first calculated (see Materials and Methods) for all possible 8, 9, 10 and 11 amino acid peptides for which structural data were available (16,604 possible CD8⁺ T cell epitopes). Those peptides with an epitope network score >3.00 were further down-selected, which was a similar cutoff for protective epitopes identified in HIV (Gaiha et al., 2019). By applying the NetMHCpan4.1 epitope prediction algorithm (http://www.cbs.dtu.dk/services/NetMHCpan/) to these epitopes, putative binders were identified for each of 18 HLA class I alleles (311 in total), which provide >99% coverage of the global population (A *0101, A *0201, A *0301, A *2402, B*0702, B*0801, B*1402, B*1501, B*2705, B*3501, B*3901, B*4001, B*4402, B*5201, B*5701, B*5801, B*8101 and Cw*0701) (Sette and Sidney, 1999; Sidney et al., 2008). It was then confirmed whether these epitopes could bind and stabilize HLA class I alleles using a newly established HLA class I-peptide stability assay which leverages CRISPR/Cas9 edited transporter associated with antigen processing (TAP)-deficient mono-allelic HLA class I-expressing cell lines. HLA class I-peptide stability plays a key role in defining immunodominance hierarchies across the HIV proteome and outperforms standard binding affinity. Thus, epitopes that achieved at least 50% relative HLA stabilization to an HLA-matched immunodominant HIV epitope were considered to be promising SARS-CoV-2 T cell immunogens given the immunogenicity of HIV epitopes that reached this threshold (Streeck et al., 2009).

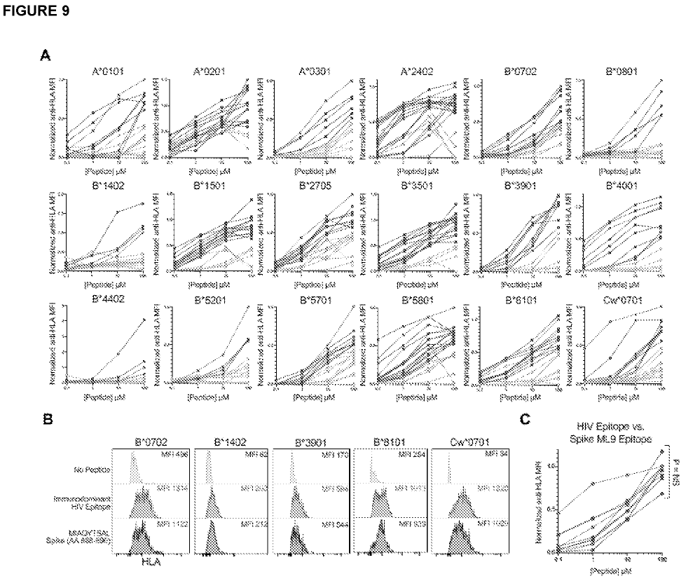

For assessments of HLA class I-peptide stability, TAP-deficient cells were incubated for 18h at 26° C. in the presence of peptide prior to a 2h incubation at 37° C. Stable HLA class I-peptide complexes were then detected on the cell surface using an anti-HLA antibody and the change in anti-HLA mean fluorescence intensity (MFI) from baseline was used to measure the degree of HLA molecule stabilization. As a representative example, TAP-deficient HLA-A*0301 mono-allelic cells were incubated with the well-defined immunodominant HIV A*0301-restricted RK9 epitope (Gag p17 20-28) and 15 highly networked SARS-CoV-2 peptides that were predicted to bind to HLA-A*0301 by NetMHCPan 4.1 and found five epitopes that successfully stabilized HLA-A*0301 on the cell surface at a level >50% of HIV RK9 (FIG. 9B). This assay was performed for all 311 predicted epitopes across the 18 TAP-deficient HLA class I-expressing cell lines at increasing peptide concentrations (0.1-100 µM) and detected >50% relative HLA class I stabilization for 109 epitopes, of which 56 were derived from SARS-CoV-2 non-structural proteins and 53 were derived from structural proteins and the accessory protein ORF3a (FIG. 8C, FIG. 9A). Representative examples of HLA stabilizing epitopes for HLA-A*0301 include the RK11 epitope from NSP16 (ORFla 6864-6874) and KR10 epitope from Spike (310-319), both of which occupy centrally located positions in their respective viral proteins (FIG. 8D). Several peptides which stabilized a number of HLA class I alleles were identified, such as Spike epitope MIAQTYSAL (869-877) (FIGS. 9B and 9C, Table 3), which has been shown to induce T cell reactivity in distinct cohorts of convalescent COVID-19 individuals (Peng et al., 2020).

TABLE 3

List of SARS-CoV-2 epitopes tested by HLA class I-peptide stability assay

| HLA | Epitope Code | Epitope Sequence | Gene | Amino Acid | Protein | Protein Region | Network Score | Net MHC Binding | 50% Experimental Binder |
|---|---|---|---|---|---|---|---|---|---|
| B*4001 | B40 COVID 9 | AGEAANF CAL | ORF1ab | 1704-1713 | nsp3 | PLPro | 3.892642725 | 0.7588 | YES |
| A*0201 | A2 COVID 4 | ALNTLVK QL | spike | 958-966 | spike | S2 domain, HR1 | 3.54409043 | 0.6159 | YES |
| Cw*07 | CW07 COVID 11 | AMPNMLR IM | ORF1ab | 5018-5026 | nsp12 | Polymerase, palm-fingers domain interface | 4.752547417 | 1.7798 | YES |
| B*5801 | B58 COVID 11 | APGTAVL ROW | ORF1ab | 6878-6887 | nsp16 | | 6.418435098 | 1.1605 | YES |
| B*0702 | B7 COVID 17 | APSASAFF | nucleocapsid | 309-316 | nucleocapsid | CTD (dimerizatio n domain) | 5.86470604 | 0.9508 | YES |
| B*0702 | B7 COVID 4 | APSASAFF GM | nucleocapsid | 309-318 | Nucleocapsi d | CTD (dimerizatio n domain) | 5.431728605 | 0.4539 | YES |
| B*8101 | B81 COVID 2 | APSASAFF GM | nucleocapsid | 309-318 | nucleocapsid | CTD (dimerizatio n domain) | 5.431728605 | 0.4461 | YES |
| A*0201 | A2 COVID 17 | AQFAPSAS A | nucleocapsid | 306-314 | nucleocapsid | CTD (dimerizatio n domain) | 6.665747133 | 1.2639 | YES |
| B*5201 | B52 COVID 15 | AQFAPSAS A | nucleocapsid | 306-314 | nucleocapsid | CTD (dimerizatio n domain) | 6.665747133 | 0.7768 | YES |
| B*1501 | B15 COVID 8 | AQVLSEM VM | ORF1ab | 5053-5061 | nsp12 | Polymerase, fingers domain | 6.316407487 | 0.3253 | YES |
| B*2705 | B27 COVID 14 | ARTRSMW SF | membrane | 104-112 | membrane | | 3.152961685 | 0.0737 | YES |
| A*2402 | A24 COVID 9 | AWPLIVTA L | ORF1ab | 4124-4132 | nsp8 | | 3.897186827 | 0.3387 | YES |
| B*1402 | B14 COVID 1 | DRAMPNM L | ORF1ab | 5016-5023 | nsp12 | Polymerase, palm-fingers domain interface | 3.592955727 | 0.1776 | YES |
| B*3901 | B39 COVID 10 | DRAMPNM L | ORF1ab | 5016-5023 | nsp12 | Polymerase, palm-fingers domain interface | 3.592955727 | 0.7157 | YES |
| B*0801 | B8 COVID 5 | FCYMHHM EL | ORF1ab | 3422-3430 | nsp5 | 3CLPro | 4.363542833 | 0.5195 | YES |
| A*0201 | A2 COVID 13 | FELLHAPA TV | spike | 515-524 | spike | S1 domain | 3.430839571 | 0.3513 | YES |
| B*4001 | B40 COVID 13 | FELLHAPA TV | spike | 515-524 | spike | S1 domain | 3.430839571 | 0.811 | YES |
| B*3501 | B35 COVID 6 | FPQSAPHG V | spike | 1052-1060 | spike | S2 domain | 4.20511584 | 0.3956 | YES |
| B*0702 | B7 COVID 14 | FPQSAPHG VVF | spike | 1052-1062 | spike | S2 domain | 3.182595915 | 0.3862 | YES |
| B*3501 | B35 COVID 15 | FPQSAPHG VVF | spike | 1052-1062 | spike | S2 domain | 3.182595915 | 0.0909 | YES |
| B*4001 | B40 COVID 3 | GEAANFC AL | ORF1ab | 1705-1713 | nsp3 | PLPro | 3.749100884 | 0.0827 | YES |
| B*4402 | B44 COVID 10 | GEAANFC AL | ORF1ab | 1705-1713 | nsp3 | PLPro | 3.749100884 | 0.828 | YES |
| B*3901 | B39 COVID 12 | GHLRIAGH HL | membrane | 147-156 | membrane | | 4.318975716 | 0.9593 | YES |
| A*0301 | A3 COVID 12 | GNYQCGH YK | ORF1ab | 1829-1837 | nsp3 | PLPro | 5.788941644 | 1.3875 | YES |
| B*5701 | B57 COVID 4 | GTAVLRQ W | ORF1ab | 6879-6886 | nsp16 | | 7.793849325 | 0.1128 | YES |
| B*5801 | B58 COVID 6 | GTAVLRQ W | ORF1ab | 6879-6886 | nsp16 | | 7.793849325 | 0.31 | YES |
| B*5801 | B58 COVID 12 | GVDIAANT VIW | ORF1ab | 6529-6539 | nsp15 | Middle domain | 8.025100372 | 1.2188 | YES |
| B*5701 | B57 COVID 16 | GVFVSNG THW | spike | 1093-1102 | spike | S2 domain | 3.151059863 | 0.1631 | YES |
| B*5801 | B58 COVID 18 | GVFVSNG THW | spike | 1093-1102 | spike | S2 domain | 3.151059863 | 0.2023 | YES |
| B*5801 | B58 COVID 7 | IAANTVIW | ORF1ab | 6531-6538 | nsp15 | Middle domain | 7.023927155 | 0.2784 | YES |
| A*0301 | A3 COVID 4 | ILPVSMTK | spike | 726-733 | spike | S2 domain | 4.519834608 | 0.5464 | YES |
| B*3501 | B35 COVID 7 | IPTITQMN L | ORF1ab | 4928-4936 | nsp12 | Polymerase, fingers domain | 6.4008422 | 0.4547 | YES |
| B*8101 | B81 COVID 3 | IPTITQMN L | ORF1ab | 4928-4936 | nsp12 | Polymerase, fingers domain | 6.4008422 | 0.0575 | YES |
| A*2402 | A24 COVID 16 | IPYNSVTS SI | ORF3a | 158-167 | protein 3a | | 3.414312272 | 0.5731 | YES |
| B*0702 | B7 COVID 15 | IPYNSVTS SI | ORF3a | 158-167 | protein 3a | | 3.41431227 | 0.4132 | YES |
| B*8101 | B81 COVID 13 | IPYNSVTS SI | ORF3a | 158-167 | protein 3a | | 3.414312272 | 0.2624 | YES |
| A*2402 | A24 COVID 2 | IYQTSNFR V | spike | 312-320 | spike | RBD, S1 domain | 5.553823153 | 0.2183 | YES |
| B*1501 | B15 COVID 1 | KGIYQTSN F | spike | 310-318 | spike | RBD, S1 domain | 5.895892813 | 0.9647 | YES |
| B*5801 | B58 COVID 2 | KGIYQTSN F | spike | 310-318 | Spike | RBD, S1 domain | 5.895892813 | 0.7466 | YES |
| A*0301 | A3 COVID 15 | KGIYQTSN FR | spike | 310-319 | spike | S1 domain, RBD (N-terminal end) | 3.468528925 | 0.4209 | YES |
| A*0201 | A2 COVID 1 | KLNDLCFT NV | spike | 386-395 | spike | RBD, S1 domain | 5.335500456 | 0.2287 | YES |
| B*1501 | B15 COVID 14 | KLNDLCFT NVY | spike | 386-396 | spike | RBD, S1 domain | 3.518474412 | 0.7267 | YES |
| B*1501 | B15 COVID 6 | KQASLNG VTL | ORF1ab | 6611-6620 | nsp15 | Middle domain | 5.640327504 | 0.6642 | YES |
| B*2705 | B27 COVID 2 | KRNVIPTIT QM | ORF1ab | 4924-4934 | nsp12 | Polymerase, fingers domain | 4.616354565 | 0.1764 | YES |
| B*2705 | B27 COVID 12 | KRVDFCG K | spike | 1038-1045 | spike | S2 domain | 7.903753048 | 1.8066 | YES |
| B*2705 | B27 COVID 1 | KRVDFCG KGY | spike | 1038-1047 | spike | S2 domain | 7.874851134 | 0.1025 | YES |
| B*5801 | B58 COVID 19 | KTSVDCT MY | spike | 733-741 | spike | S2 domain | 4.349010357 | 0.5691 | YES |
| A*2402 | A24 COVID 3 | KWADNNC YL | ORF1ab | 1668-1676 | nsp3 | PLPro | 12.19840721 | 0.6587 | YES |
| B*1501 | B15 COVID 10 | LLKSAYEN F | ORF1ab | 1130-1138 | nsp3 | ADP Ribose Phosphatase | 4.028688463 | 0.1258 | YES |
| A*0201 | A2 COVID 7 | LLTLQQIE L | ORF1ab | 1680-1688 | nsp3 | PLPro | 4.945425527 | 0.738 | YES |
| A*0201 | A2 COVID 12 | LLYDANY FL | ORF3a | 139-147 | protein 3a | | 3.861809732 | 0.0167 | YES |
| B*0702 | B7 COVID 18 | LPVSMTKT SV | spike | 727-736 | spike | S2 domain | 3.713431205 | 0.6784 | YES |
| B*8101 | B81 COVID 15 | LPVSMTKT SV | spike | 727-736 | spike | S2 domain | 3.71343121 | 0.7889 | YES |
| Cw*07 | CW07 COVID 16 | LRIAGHHL | membrane | 149-156 | membrane | | 3.904974107 | 0.8131 | YES |
| B*2705 | B27 COVID 4 | LRQWLPT GTL | ORF1ab | 6884-6893 | nsp16 | | 7.829281412 | 0.6603 | YES |
| B*3901 | B39 COVID 7 | LRQWLPT GTL | ORF1ab | 6883-6892 | nsp16 | | 7.829281412 | 0.9791 | YES |
| B*2705 | B27 COVID 6 | LRQWLPT GTLL | ORF1ab | 6883-6893 | nsp16 | | 8.25791002 | 0.9493 | YES |
| B*0702 | B7 COVID 16 | MIAQYTSA L | spike | 869-877 | spike | S2 domain | 3.442891968 | 0.4728 | YES |
| B*1402 | B14 COVID 11 | MIAQYTSA L | spike | 869-877 | spike | S2 domain | 3.442891968 | 0.237 | YES |
| B*3501 | B35 COVID 17 | MIAQYTSA L | spike | 869-877 | spike | S2 domain | 3.442891968 | 0.7641 | YES |
| B*3901 | B39 COVID 13 | MIAQYTSA L | spike | 869-877 | spike | S2 domain | 3.442891968 | 0.9927 | YES |
| B*8101 | B81 COVID 12 | MIAQYTSA L | spike | 869-877 | spike | S2 domain | 3.44289197 | 0.2294 | YES |
| Cw*07 | CW07 COVID 17 | MIAQYTSA L | spike | 869-877 | spike | S2 domain | 3.442891968 | 0.88 | YES |
| B*0702 | B7 COVID 2 | MPILTLTR | ORF1ab | 4635-4644 | nsp12 | N-terminal extension | 6.249572175 | 0.179 | YES |
| B*8101 | B81 COVID | MPILITLTR | ORF1ab | 4635-4644 | nsp12 | N-terminal | 6.249572175 | 0.0892 | YES |
| | 5 | AL | | | | extension | | | |
| A*0101 | A1 COVID 6 | MVMCGGS LY | ORF1ab | 5059-5067 | nsp12 | Polymerase, fingers domain | 6.91349846 | 0.5057 | YES |
| B*1501 | B15 COVID 4 | MVMCGGS LY | ORF1ab | 5059-5067 | nsp12 | Polymerase, fingers domain | 9.33811313 | 0.3128 | YES |
| B*3501 | B35 COVID 3 | MVMCGGS LY | ORF1ab | 5059-5067 | nsp12 | Polymerase, fingers domain | 9.33811313 | 0.2355 | YES |
| A*0201 | A2 COVID 11 | MVMCGGS LYV | ORF1ab | 5059-5068 | nsp12 | Polymerase, fingers domain | 11.3950339 | 1.0226 | YES |
| A*2402 | A24 COVID 19 | MWSFNPET NIL | membrane | 109-119 | membrane | | 7.790642112 | 1.3264 | YES |
| B*3501 | B35 COVID 11 | NASSSEAF L | ORF1ab | 6997-7005 | nsp16 | | 6.141646034 | 1.9686 | YES |
| B*8101 | B81 COVID 14 | NPLLYDAN YFL | ORF3a | 137-147 | protein 3a | | 3.651957922 | 0.5944 | YES |
| A*0101 | A1 COVID 16 | NSSPDDQI GY | nucleocapsi d | 78-87 | nucleocapsid | NTD (RNA-binding domain) | 3.46476264 | 0.2797 | YES |
| A*0101 | A1 COVID 2 | NSSPDDQI GYY | nucleocapsi d | 78-88 | nucleocapsid | NTD (RNA-binding domain) | 4.27459417 | 0.0281 | YES |
| B*3501 | B35 COVID 2 | NVIPTITQM | ORF1ab | 4926-4934 | nsp12 | Polymerase, fingers domain | 6.00451691 | 0.2078 | YES |
| A*0101 | A1 COVID 14 | PDDQIGYY | nucleocapsi d | 81-88 | nucleocapsid | NTD (RNA-binding domain) | 4.462600661 | 1.9005 | YES |
| B*5801 | B58 COVID 13 | PGTAVLRQ W | ORF1ab | 6879-6887 | nsp16 | | 6.988429462 | 1.1648 | YES |
| A*0101 | A1 COVID 3 | PLLTDEMI AQY | spike | 863-873 | spike | S2 domain | 3.888052107 | 0.1254 | YES |
| A*2402 | A24 COVID 1 | QFAPSASA F | Nucleocaps id | 307-315 | Nucleocapsid | CTD (dimerization domain) | 7.130441769 | 0.189 | YES |
| B*1501 | B15 COVID 3 | QFAPSASA F | Nucleocaps id | 307-315 | Nucleocapsid | CTD (dimerization domain) | 7.130441769 | 0.8454 | YES |
| B*3501 | B35 COVID 10 | QFAPSASA F | Nucleocaps id | 307-315 | Nucleocapsid | CTD (dimerization domain) | 7.130441769 | 0.7964 | YES |
| A*2402 | A24 COVID 13 | QFAPSASA FF | nucleocapsi d | 307-316 | nucleocapsid | CTD (dimerization domain) | 6.41048592 | 0.2408 | YES |
| B*0702 | B7 COVID 8 | QPGQTFSV L | ORF1ab | 3370-3378 | nsp5 | 3CLPro | 3.44423533 | 0.0454 | YES |
| B*3501 | B35 COVID 14 | QPTESIVRF | spike | 321-329 | spike | S1 domain, RBD | 3.608489479 | 0.0311 | YES |
| B*1501 | B15 COVID 11 | QTFSVLAC Y | ORF1ab | 3373-3381 | nsp5 | 3CLPro | 4.894165401 | 1.2948 | YES |
| B*5701 | B57 COVID 3 | QVNGLTSI KW | ORF1ab | 1660-1669 | nsp3 | PLPro | 9.760680774 | 0.3292 | YES |
| B*5801 | B58 COVID 4 | QVNGLTSI KW | ORFlab | 1660-1669 | nsp3 | PLPro | 9.760680774 | 0.3082 | YES |
| A*2402 | A24 COVID 5 | QWLPTGTL L | ORF1ab | 6886-6894 | nsp16 | | 7.142464433 | 0.2013 | YES |
| B*1501 | B15 COVID 2 | RGVYYPD KVF | spike | 34-43 | spike | S1 domain | 4.007433807 | 0.5979 | YES |
| B*5701 | B57 COVID 18 | RLFARTRS MW | membrane | 101-110 | membrane | | 4.26475289 | 0.4384 | YES |
| B*5201 | B52 COVID 2 | RQLLFVVE V | ORF1ab | 4860-4868 | nsp12 | Polymerase, fingers domain | 5.149893163 | 0.0948 | YES |
| B*1501 | B15 COVID 9 | RQWLPTGT L | ORF1ab | 6884-6892 | nsp16 | | 6.561670968 | 0.4568 | YES |
| B*3901 | B39 COVID 2 | RQWLPTGT L | ORF1ab | 6885-6893 | nsp16 | | 6.561670968 | 0.4267 | YES |
| B*4001 | B40 COVID 6 | RQWLPTGT L | ORF1ab | 6885-6893 | nsp16 | | 6.561670968 | 0.6831 | YES |
| A*2402 | A24 COVID 4 | RQWLPTGT LL | ORF1ab | 6885-6894 | nsp16 | | 7.04096948 9 | 0.3766 | YES |
| B*1501 | B15 COVID 7 | RQWLPTGT LL | ORF1ab | 6884-6893 | nsp16 | | 7.04096948 9 | 0.8293 | YES |
| B*4001 | B40 COVID 7 | RQWLPTGT LL | ORF1ab | 6885-6894 | nsp16 | | 7.04096948 9 | 1.1573 | YES |
| B*2705 | B27 COVID 16 | RRGPEQTQ GNF | nucleocapsi d | 277-287 | nucleocapsid | CTD (dimerization domain) | 5.12220211 2 | 0.4384 | YES |
| Cw*07 | CW07 COVID 19 | RRGPEQTQ GNF | nucleocapsi d | 277-287 | nucleocapsid | CTD (dimarization domain) | 5.12220211 2 | 1.3719 | YES |
| B*5701 | B57 COVID 19 | RTRSMWSF | membrane | 105-112 | membrane | | 3.17132531 | 0.4391 | YES |
| A*0301 | A3 COVID 3 | RVIHFGAG SDK | ORF1ab | 6864-6874 | nsp16 | | 3.71700215 4 | 0.4287 | YES |
| B*5801 | B58 COVID 1 | RVQPTESIV RF | spike | 319-329 | Spike | RBD, S1 domain | 7.46260527 9 | 1.0993 | YES |
| B*5701 | B57 COVID 7 | SALNHTKK W | ORF1ab | 1648-1656 | nsp3 | PLPro | 4.76369510 9 | 0.0172 | YES |
| B*5801 | B58 COVID 10 | SALNHTKK W | ORF1ab | 1648-1656 | nsp3 | PLPro | 4.76369510 9 | 0.0282 | YES |
| B*4001 | B40 COVID 2 | SEMVMCG GSL | ORF1ab | 5057-5066 | nsp12 | Polymerase, fingers domain | 8.20916987 9 | 0.3848 | YES |
| B*4402 | B44 COVID 4 | SEMVMCG GSL | ORF1ab | 5057-5066 | nsp12 | Polymerase, fingers domain | 8.20916987 9 | 0.9289 | YES |
| B*4001 | B40 COVID 8 | SEYTGNYQ C | ORF1ab | 1825-1833 | nsp3 | PLPro | 5.67781337 8 | 0.52 | YES |
| A*2402 | A24 COVID 15 | SFNPETNIL | membrane | 111-119 | membrane | | 6.47765125 9 | 0.4405 | YES |
| Cw*07 | CW07 COVID 14 | SFNPETNIL | membrane | 111-119 | membrane | | 6.47765125 9 | 0.5018 | YES |
| A*2402 | A24 COVID 17 | SFNPETNIL L | membrane | 111-120 | membrane | | 4.19166408 | 0.7292 | YES |
| B*0801 | B8 COVID 2 | SIKNFKSVL | ORF1ab | 5171-5179 | nsp12 | Polymerase, palm domain | 3.98654166 9 | 0.1063 | YES |
| B*1501 | B15 COVID 12 | SIKWADNN CY | ORF1ab | 1666-1675 | nsp3 | PLPro | 7.65885133 1 | 1.1624 | YES |
| A*0201 | A2 COVID 15 | SMWSFNPE T | membrane | 108-116 | membrane | | 3.64068450 9 | 0.4543 | YES |
| B*3501 | B35 COVID 13 | SPDDQIGY Y | nucleocapsi d | 80-88 | nucleocapsid | NTD (RNA-binding domain) | 3.26855248 5 | 0.0261 | YES |
| A*0101 | A1 COVID 1 | SSPDDQIG YY | nucleocapsi d | 79-88 | nucleocapsid | NTD (RNA-binding domain) | 3.45045522 | 0.0095 | YES |
| B*3501 | B35 COVID 4 | SSPDDQIG YY | Nucleocaps id | 79-88 | Nucleocapsid | NTD (RNA-binding domain) | 3.84051256 4 | 0.2958 | YES |
| B*4001 | B40 COVID 1 | TEILPVSM | spike | 724-731 | Spike | S2 domain | 3.77539378 9 | 0.3783 | YES |
| B*0801 | B8 COVID 14 | TILTRPLL | membrane | 127-134 | membrane | | 3.11110075 9 | 0.2706 | YES |
| B*3501 | B35 COVID 16 | TSNEVAVL Y | spike | 604-612 | spike | S1 domain | 3.14703563 5 | 0.1757 | YES |
| B*5801 | B58 COVID 17 | TSNEVAVL Y | spike | 604-612 | spike | S1 domain | 3.14703563 5 | 0.1314 | YES |
| B*3501 | B35 COVID 1 | TTLPVNVA F | ORF1ab | 6499-6507 | nsp15 | N-terminal domain | 3.73869781 6 | 0.186 | YES |
| B*5801 | B58 COVID 9 | VAPGTAVL RQW | ORF1ab | 6877-6887 | nsp16 | | 6.29139948 8 | 0.6269 | YES |
| B*8101 | B81 COVID 11 | VIPTITQMN L | ORF1ab | 4927-4936 | nsp12 | Polymerase, fingers domain | 5.84840291 3 | 1.0201 | YES |
| A*0201 | A2 COVID 3 | VLNDILSR L | spike | 976-984 | spike | S2 domain | 3.80495392 6 | 0.0356 | YES |
| A*0201 | A2 COVID 5 | VMCGGSL YV | ORF1ab | 5060-5068 | nsp12 | Polymerase, fingers domain | 7.69868454 7 | 0.3164 | YES |
| A*0301 | A3 COVID 2 | VMCGGSL YVK | ORF1ab | 5060-5069 | nsp12 | Polymerase, fingers domain | 7.34813110 1 | 0.3897 | YES |
| B*5801 | B58 COVID 5 | VNGLTSIK W | ORF1ab | 1661-1669 | nsp3 | PLPro | 9.43358372 5 | 0.7786 | YES |
| B*3501 | B35 COVID 5 | VPVVDSYY | ORF1ab | 4624-4631 | nsp12 | | 5.06773537 7 | 0.3298 | YES |
| B*0801 | B8 COVID 17 | VSMTKTSV | spike | 729-736 | spike | S2 domain | 3.41332465 9 | 0.8462 | YES |
| B*5701 | B57 COVID 8 | VTANVNA LL | ORF1ab | 5093-5101 | nsp12 | Polymerase, palm domain | 4.54913575 6 | 0.472 | YES |
| B*8101 | B81 COVID 6 | VVNAANY YL | ORF1ab | 1057-1065 | nsp3 | ADP Ribose Phosphatase | 4.08310717 4 | 0.7781 | YES |
| B*4402 | B44 COVID 12 | YDANYFLC W | ORF3a | 141-149 | protein 3a | | 7.60089485 4 | 0.7892 | YES |
| A*0201 | A2 COVID 14 | YHLMSFPQ SA | spike | 1047-1056 | spike | S2 domain | 4.45063696 | 0.4427 | YES |
| A*0201 | A2 COVID 6 | YLATALLT L | ORF1ab | 1675-1683 | nsp3 | PLPro | 6.22114874 | 0.0403 | YES |
| B*3901 | B39 COVID 1 | YLATALLT L | ORF1ab | 1675-1683 | nsp3 | PLPro | 6.22114874 | 0.2988 | YES |
| Cw*07 | CW07 COVID 6 | YLATALLTL | OPF1ab | 1675-1683 | nsp3 | PTPro | 6.22114874 | 0.481 | YES |
| B*0702 | B7 COVID 10 | YPKCDRA M | ORF1ab | 5012-5019 | nsp12 | Polymerase, palm domain | 5.03086931 8 | 0.689 | YES |
| B*5201 | B52 COVID 7 | YQCGHYK HI | ORF1ab | 1831-1839 | nsp3 | PLPro | 7.95176437 9 | 0.5338 | YES |
| B*3901 | B39 COVID 11 | YQDVNCTE V | spike | 612-620 | spike | S1 domain | 3.63891862 | 0.8461 | YES |
| B*1402 | B14 COVID 3 | YRFNGIGV | spike | 904-911 | Spike | HRI, S2 domain | 3.57747533 4 | 0.3982 | YES |
| B*2705 | B27 COVID 3 | YRFNGIGV | spike | 904-911 | spike | HRI, S2 domain | 3.57747533 4 | 0.5346 | YES |
| B*3901 | B39 COVID 3 | YRFNGIGV | spike | 904-911 | Spike | HRI, S2 domain | 3.57747533 4 | 0.5151 | YES |
| Cw*07 | CW07 COVID 9 | YRFNGIGV | spike | 904-911 | spike | S2 domain | 3.57747533 4 | 0.6979 | YES |
| A*0101 | A1 COVID 4 | YTGNYQC GHY | ORF1ab | 1827-1836 | nsp3 | PLPro | 6.60569764 2 | 0.2499 | YES |
| A*2402 | A24 COVID 18 | YYPDKVFR SSV | spike | 37-47 | spike | S1 domain | 3.28160933 5 | 0.9019 | YES |
| A*2402 | A24 COVID 6 | YYSLLMPI L | ORF1ab | 4630-4638 | nsp12 | N-terminal extension | 5.43104964 9 | 0.3998 | YES |
| Cw*07 | CW07 COVID 3 | YYSLLMPI L | ORF1ab | 4630-4638 | nsp12 | Polymerase, N-terminal extension | 5.43104964 9 | 0.3968 | YES |
| A*2402 | A24 COVID 8 | YYSLLMPI LTL | ORF1ab | 4630-4640 | nsp12 | N-terminal extension | 5.30683528 7 | 0.9167 | YES |

Alignment of highly networked, HLA stabilizing epitopes with sequences of the UK, South African and Brazilian SARS-CoV-2 variants revealed that 91.7% of epitopes had no mutations and 100% of epitopes had ≤1 amino acid variant (FIGS. 8E and 8F, Table 5).

Table 5 depicts epitopes tested by HLA class I-peptide stability assay and delineates those that have at least 50% relative HLA class I stabilization in comparison to an immunodominant HIV epitope. Further depicted is alignment of highly networked, HLA stabilizing epitopes with homologous sequences in the UK, South African and Brazilian SARS-CoV-2 variants which reveals that 91.7% of stabilizing epitopes had no mutations and 100% of stabilizing epitopes had ≤1 amino acid variant (see also FIGS. 8E and 8F).

TABLE 5

Sequences of Highly Networked, HLA stabilizing SARS-CoV-2 Epitopes in the B1.1.7, B.1.351 and P.1 Variants

| Epitope Code | WT Sequence | B.1.1.7 | B.1.351 | P.1 | Protein | Protein Region | Amino Acid | Network Score |
|---|---|---|---|---|---|---|---|---|
| A1 COVID 1 | SSPDDQIGYY | SSPDDQIGYY | SSPDDQIGYY | SSRDDQIGYY | nucleocapsid | NTD (RNA-binding domain) | 79-88 | 3.45045522 |
| A1 COVID 2 | NSSPDDQIGYY | NSSPDDQIGYY | NSSPDDQIGYY | NSSRDDQIGY Y | nucleocapsid | NTD ( RNA-binding domain) | 78-88 | 4.27459417 |
| A1 COVID 3 | PLLTDEMIAQY | PLLTDEMIAQY | PLLTDEMIAQY | PLLTDEMIAQY | spike | S2 domain | 863-873 | 3.888052107 |
| A1 COVID 4 | YTGNYQCGHY | YTGNYQCGHY | YTGNYQCGHY | YTGNYQCGHY | nsp3 | PLPro | 1827-1836 | 6.605697642 |
| A1 COVID 6 | MVMCGGSLY | MVMCGGSLY | MVMCGGSLY | MVMCGGSLY | nsp12 | Polymerase, fingers domain | 5059-5067 | 6.91349846 |
| A1 COVID 14 | PDDQIGYY | PDDQIGYY | PDDQIGYY | RDDQIGYY | nucleocapsid | NTD (RNA-binding domain) | 81-88 | 4.462600661 |
| A1 COVID 16 | NSSPDDQIGY | NSSPDDQIGY | NSSPDDQIGY | NSSRDDQIGY | nucleocapsid | NTD (RNA-binding domain) | 78-87 | 3.46476264 |
| A2 COVID 1 | KLNDLCFTNV | KLNDLCFTNV | KLNDLCFTNV | KLNDLCFTNV | spike | RBD, S1 domain | 386-395 | 5.335500456 |
| A2 COVID 3 | VLNDILSRL | VLNDILARL | VLNDILSRL | VLNDILSRL | spike | S2 domain | 976-984 | 3.804953926 |
| A2 COVID 4 | ALNTLVKQL | ALNTLVKQL | ALNTLVKQL | ALNTLVKQL | spike | S2 domain, HR1 | 958-966 | 3.54409043 |
| A2 COVID 5 | QTFSVLACY | QTFSVLACY | QTFSVLACY | QTFSVLACY | nsp5 | 3CLPro | 3373-3381 | 4.894165401 |
| A2 COVID 6 | YLATALLTL | YLATALLTL | YLATALLTL | YLATALLTL | nsp3 | PLPro | 1675-1683 | 6.22114874 |
| A2 COVID 7 | LLTLQQIEL | LLTLQQIEL | LLTLQQIEL | LLTLQQIEL | nsp3 | PLPro | 1680-1688 | 4.945425527 |
| A2 COVID 11 | MVMCGGSLYV | MVMCGGSLYV | MVMCGGSLYV | MVMCGGSLYV | nsp12 | Polymerase, fingers domain | 5059-5068 | 11.3950339 |
| A2 COVID 12 | LLYDANYFL | LLYDANYFL | LLYDANYFL | LLYDANYFL | protein 3a | | 139-147 | 3.861809732 |
| A2 COVID 13 | FELLHAPATV | FELLHAPATV | FELLHAPATV | FELLHAPATV | spike | S1 domain | 515-524 | 3.430839571 |
| A2 COVID 14 | YHLMSFPQSA | YHLMSFPQSA | YHLMSFPQSA | YHLMSFPQSA | spike | S2 domain | 1047-1056 | 4.45063696 |
| A2 COVID 15 | SMWSFNPET | SMWSFNPET | SMWSFNPET | SMWSFNPET | membrane | | 108-116 | 3.640684509 |
| A2 COVID 17 | AQFAPSASA | AQFAPSASA | AQFAPSASA | AQFAPSASA | nucleocapsid | CTD (dimerization domain) | 306-314 | 6.665747133 |
| A3 COVID 2 | VMCGGSLYVK | VMCGGSLYVK | VMCGGSLYVK | VMCGGSLYVK | nsp12 | Polymerase, fingers domain | 5060-5069 | 7.348131101 |
| A3 COVID 3 | RVIHFGAGSDK | RVIHFGAGSDK | RVIHFGAGSDK | RVIHFGAGSD K | nsp16 | | 6864-6874 | 3.717002154 |
| A3 COVID 4 | ILPVSMTK | ILPVSMTK | ILPVSMTK | ILPVSMTK | spike | S2 domain | 726-733 | 4.519834608 |
| A3 COVID 12 | GNYQCGHYK | GNYQCGHYK | GNYQCGHYK | GNYQCGHYK | nsp3 | PLPro | 1829-1837 | 5.788941644 |
| A3 COVID 15 | KGIYQTSNFR | KGIYQTSNFR | KGIYQTSNFR | KGIYQTSNFR | spike | S1 domain, RBD (N-terminal end) | 310-319 | 3.468528925 |
| A24 COVID 1 | QFAPSASAF | QFAPSASAF | QFAPSASAF | QFAPSASAF | nucleocapsid | CTD (dimerization domain) | 307-315 | 7.130441769 |
| A24 COVID 2 | IYQTSNFRV | IYQTSNFRV | IYQTSNFRV | IYQTSNFRV | spike | RBD, S1 domain | 312-320 | 5.553823153 |
| A24 COVID 3 | KWADNNCYL | KWADNNCYL | KWADNNCYL | KWADNNCYL | nsp3 | PLPro | 1668-1676 | 12.19840721 |
| A24 COVID 4 | RQWLPTGTLL | RQWLPTGTLL | RQWLPTGTLL | RQWLPTGTLL | nsp16 | | 6885-6894 | 7.040969489 |
| A24 COVID 5 | QWLPTGTLL | QWLPTGTLL | QWLPTGTLL | QWLPTGTLL | nsp16 | | 6886-6894 | 7.142464433 |
| A24 COVID 6 | YYSLLMPIL | YYSLLMPIL | YYSLLMPIL | YYSLLMPIL | nsp12 | N-terminal extension | 4630-4638 | 5.431049649 |
| A24 COVID 8 | YYSLLMPILTL | YYSLLMPILTL | YYSLLMPILTL | YYSLLMPILTL | nsp12 | N-terminal extension | 4630-4640 | 5.306835287 |
| A24 COVID 9 | AWPLIVTAL | AWPLIVTAL | AWPLIVTAL | AWPLIVTAL | nsp8 | | 4124-4132 | 3.897186827 |
| A24 COVID 13 | QFAPSASAFF | QFAPSASAFF | QFAPSASAFF | QFAPSASAFF | nucleocapsid | CTD (dimerization domain) | 307-316 | 6.41048592 |
| A24 COVID 15 | SFNPETNIL | SFNPETNIL | SFNPETNIL | SFNPETNIL | membrane | | 111-119 | 6.477651259 |
| A24 COVID 16 | IPYNSVTSSI | IPYNSVTSSI | IPYNSVTSSI | IPYNSVTSSI | protein 3a | | 158-167 | 3.414312272 |
| A24 COVID 17 | SFNPETNILL | SFNPETNILL | SFNPETNILL | SFNPETNILL | membrane | | 111-120 | 4.19166408 |
| A24 COVID 18 | YYPDKVFRSSV | YYPDKVFRSSV | YYPDKVFRSSV | YYPDKVFRSS V | spike | S1 domain | 37-47 | 3.281609335 |
| A24 COVID 19 | MWSFNPETNIL | MWSFNPETNIL | MWSFNPETNIL | MWSFNPETNI L | membrane | | 109-119 | 7.790642112 |
| B7 COVID 2 | MPILTLTRAL | MPILTLTRAL | MPILTLTRAL | MPILTLTRAL | nsp12 | N-terminal extension | 4635-4644 | 6.249572175 |
| B7 COVID 4 | APSASAFFGM | APSASAFFGM | APSASAFFGM | APSASAFFGM | nucleocapsid | CTD (dimerization domain) | 309-318 | 5.431728605 |
| B7 COVID 8 | QPGQTFSVL | QPGQTFSVL | QPGQTFSVL | QPGQTFSVL | nsp5 | 3CLPro | 3370-3378 | 3.44423533 |
| B7 COVID 10 | YPKCDRAM | YPKCDRAM | YPKCDRAM | YPKCDRAM | nsp12 | Polymerase, palm domain | 5012-5019 | 5.030869318 |
| B7 COVID 14 | FPQSAPHGVVF | FPQSAPHGVVF | FPQSAPHGVVF | FPQSAPHGVV F | spike | S2 domain | 1052-1062 | 3.182595915 |
| B7 COVID 16 | MIAQYTSAL | MIAQYTSAL | MIAQYTSAL | MIAQYTSAL | spike | S2 domain | 869-877 | 3.442891968 |
| B7 COVID 17 | APSASAFF | APSASAFF | APSASAFF | APSASAFF | nucleocapsid | CTD (dimerization domain) | 309-316 | 5.86470604 |
| B7 COVID 18 | LPVSMTKTSV | LPVSMTKTSV | LPVSMTKTSV | LPVSMTKTSV | spike | S2 domain | 727-736 | 3.713431205 |
| B8 COVID 2 | SIKNFKSVL | SIKNFKSVL | SIKNFKSVL | SIKNFKSVL | nsp12 | Polymerase, palm domain | 5171-5179 | 3.986541669 |
| B8 COVID 5 | FCYMHHMEL | FCYMHHMEL | FCYMHHMEL | FCYMHHMEL | nsp5 | 3CLPro | 3422-3430 | 4.363542833 |
| B8 COVID 14 | TILTRPLL | TILTRPLL | TILTRPLL | TILTRPLL | membrane | | 127-134 | 3.111100759 |
| B8 COVID 17 | VSMTKTSV | VSMTKTSV | VSMTKTSV | VSMTKTSV | spike | S2 domain | 729-736 | 3.413324659 |
| B14 COVID 1 | DRAMPNML | DRAMPNML | DRAMPNML | DRAMPNML | nsp12 | Polymerase, palm-fingers domain interface | 5016-5023 | 3.592955727 |
| B14 COVID 3 | YRFNGIGV | YRFNGIGV | YRFNGIGV | YRFNGIGV | spike | HR1, S2 domain | 904-911 | 3.577475334 |
| B15 COVID 1 | KGIYQTSNF | KGIYQTSNF | KGIYQTSNF | KGIYQTSNF | spike | RBD, S1 domain | 310-318 | 5.895892813 |
| B15 COVID 2 | RGVYYPDKVF | RGVYYPDKVF | RGVYYPDKVF | RGVYYPDKVF | spike | S1 domain | 34-43 | 4.007433807 |
| B15 COVID 4 | MVMCGGSLY | MVMCGGSLY | MVMCGGSLY | MVMCGGSLY | nsp12 | Polymerase, fingers domain | 5059-5067 | 9.33811313 |
| B15 COVID 6 | KQASLNGVTL | KQASLNGVTL | KQASLNGVTL | KQASLNGVTL | nsp15 | Middle domain | 6611-6620 | 5.640327504 |
| B15 COVID 8 | AQVLSEMVM | AQVLSEMVM | AQVLSEMVM | AQVLSEMVM | nsp12 | Polymerase, fingers domain | 5053-5061 | 6.316407487 |
| B15 COVID 9 | RQWLPTGTL | RQWLPTGTL | RQWLPTGTL | RQWLPTGTL | nsp16 | | 6884-6892 | 6.561670968 |
| B15 COVID 10 | LLKSAYENF | LLKSAYENF | LLKSAYENF | LLKSAYENF | nsp3 | ADP Ribose Phosphatase | 1130-1138 | 4.028688463 |
| B15 COVID 11 | QTFSVLACY | QTFSVLACY | QTFSVLACY | QTFSVLACY | nsp5 | 3CLPro | 3373-3381 | 4.894165401 |
| B15 COVID 12 | SIKWADNNCY | SIKWADNNCY | SIKWADNNCY | SIKWADNNCY | nsp3 | PLPro | 1666-1675 | 7.658851331 |
| B15 COVID 14 | KLNDLCFTNVY | KLNDLCFTNVY | KLNDLCFTNVY | KLNDLCFTNV Y | spike | RBD, S1 domain | 386-396 | 3.518474412 |
| B27 COVID 1 | KRVDFCGKGY | KRVDFCGKGY | KRVDFCGKGY | KRVDFCGKGY | spike | S2 domain | 1038-1047 | 7.874851134 |
| B27 COVID 2 | KRNVIPTITQM | KRNVIPTITQM | KRNVIPTITQM | KRNVIPTITQM | nsp12 | Polymerase, fingers domain | 4924-4934 | 4.616354565 |
| B27 COVID 4 | LRQWLPTGTL | LRQWLPTGTL | LRQWLPTGTL | LRQWLPTGTL | nsp16 | | 6884-6893 | 7.829281412 |
| B27 COVID 6 | LRQWLPTGTLL | LRQWLPTGTLL | LRQWLPTGTLL | LRQWLPTGTL L | nsp16 | | 6883-6893 | 8.25791002 |
| B27 COVID 12 | KRVDFCGK | KRVDFCGK | KRVDFCGK | KRVDFCGK | spike | S2 domain | 1038-1045 | 7.903753048 |
| B27 COVID 14 | ARTRSMWSF | ARTRSMWSF | ARTRSMWSF | ARTRSMWSF | membrane | | 104-112 | 3.152961685 |
| B27 COVID 16 | RRGPEQTQGNF | RRGPEQTQGNF | RRGPEQTQGNF | RRGPEQTQG NF | nucleocapsid | CTD (dimerization domain) | 277-287 | 5.122202112 |
| B35 COVID 1 | TTLPVNVAF | TTLPVNVAF | TTLPVNVAF | TTLPVNVAF | nsp15 | N-terminal domain | 6499-6507 | 3.738697816 |
| B35 COVID 2 | NVIPTITQM | NVIPTITQM | NVIPTITQM | NVIPTITQM | nsp12 | Polymerase, fingers domain | 4926-4934 | 6.00451691 |
| B35 COVID 5 | VPVVDSYY | VPVVDSYY | VPVVDSYY | VPVVDSYY | nsp12 | | 4624-4631 | 5.067735377 |
| B35 COVID 6 | FPQSAPHGV | FPQSAPHGV | FPQSAPHGV | FPQSAPHGV | spike | S2 domain | 1052-1060 | 4.20511584 |
| B35 COVID 7 | IPTITQMNL | IPTITQMNL | IPTITQMNL | IPTITQMNL | nsp12 | Polymerase, fingers domain | 4928-4936 | 6.4008422 |
| B35 COVID 11 | NASSSEAFL | NASSSEAFL | NASSSEAFL | NASSSEAFL | nsp16 | | 6997-7005 | 6.141646034 |
| B35 COVID 13 | SPDDQIGYY | SPDDQIGYY | SPDDQIGYY | SRDDQIGYY | nucleocapsid | NTD (RNA-binding domain) | 80-88 | 3.268552485 |
| B35 COVID 14 | QPTESIVRF | QPTESIVRF | QPTESIVRF | QPTESIVRF | spike | S1 domain, RBD | 321-329 | 3.608489479 |
| B35 COVID 16 | TSNEVAVLY | TSNEVAVLY | TSNEVAVLY | TSNEVAVLY | spike | S1 domain | 604-612 | 3.147035635 |
| B39 COVID 10 | DRAMPNML | DRAMPNML | DRAMPNML | DRAMPNML | nsp12 | Polymerase, palm-fingers domain interface | 5016-5023 | 3.592955727 |
| B39 COVID 11 | YQDVNCTEV | YQDVNCTEV | YQDVNCTEV | YQDVNCTEV | spike | S1 domain | 612-620 | 3.63891862 |
| B39 COVID 12 | GHLRIAGHHL | GHLRIAGHHL | GHLRIAGHHL | GHLRIAGHHL | membrane | | 147-156 | 4.318975716 |
| B40 COVID 1 | TEILPVSM | TEILPVSM | TEILPVSM | TEILPVSM | spike | S2 domain | 724-731 | 3.775393789 |
| B40 COVID 2 | SEMVMCGGSL | SEMVMCGGSL | SEMVMCGGSL | SEMVMCGGSL | nsp12 | Polymerase, fingers domain | 5057-5066 | 8.209169879 |
| B40 COVID 3 | GEAANFCAL | GEADNFCAL | GEAANFCAL | GEAANFCAL | nsp3 | PLPro | 1705-1713 | 3.749100884 |
| B40 COVID 8 | SEYTGNYQC | SEYTGNYQC | SEYTGNYQC | SEYTGNYQC | nsp3 | PLPro | 1825-1833 | 5.677813378 |
| B40 COVID 9 | AGEAANFCAL | AGEADNFCAL | AGEAANFCAL | AGEAANFCAL | nsp3 | PLPro | 1704-1713 | 3.892642725 |
| B44 COVID 12 | YDANYFLCW | YDANYFLCW | YDANYFLCW | YDANYFLCW | protein 3a | | 141-149 | 7.600894854 |
| B52 COVID 2 | RQLLFVVEV | RQLLFVVEV | RQLLFVVEV | RQLLFVVEV | nsp12 | Polymerase, fingers domain | 4860-4868 | 5.149893163 |
| B52 COVID 7 | YQCGHYKHI | YQCGHYKHI | YQCGHYKHI | YQCGHYKHI | nsp3 | PLPro | 1831-1839 | 7.951764379 |
| B57 COVID 3 | QVNGLTSIKW | QVNGLTSIKW | QVNGLTSIKW | QVNGLTSIKW | nsp3 | PLPro | 1660-1669 | 9.760680774 |
| B57 COVID 4 | GTAVLRQW | GTAVLRQW | GTAVLRQW | GTAVLRQW | nsp16 | | 6879-6886 | 7.793849325 |
| B57 COVID 7 | SALNHTKKW | SALNHTKKW | SALNHTKNW | SALNHTKKW | nsp3 | PLPro | 1648-1656 | 4.763695109 |
| B57 COVID 8 | VTANVNALL | VTANVNALL | VTANVNALL | VTANVNALL | nsp12 | Polymerase, palm domain | 5093-5101 | 4.549135756 |
| B57 COVID 16 | GVFVSNGTHW | GVFVSNGTHW | GVFVSNGTHW | GVFVSNGTHW | spike | S2 domain | 1093-1102 | 3.151059863 |
| B57 COVID 18 | RLFARTRSMW | RLFARTRSMW | RLFARTRSMW | RLFARTRSMW | membrane | | 101-110 | 4.26475289 |
| B57 COVID 19 | RTRSMWSF | RTRSMWSF | RTRSMWSF | RTRSMWSF | membrane | | 105-112 | 3.17132531 |
| B58 COVID 1 | RVQPTESIVRF | RVQPTESIVRF | RVQPTESIVRF | RVQPTESIVRF | spike | RBD, S1 domain | 319-329 | 7.462605279 |
| B58 COVID 5 | VNGLTSIKW | VNGLTSIKW | VNGLTSIKW | VNGLTSIKW | nsp3 | PLPro | 1661-1669 | 9.433583725 |
| B58 COVID 7 | IAANTVIW | IAANTVIW | IAANTVIW | IAANTVIW | nsp15 | Middle domain | 6531-6538 | 7.023927155 |
| B58 COVID 9 | VAPGTAVLRQW | VAPGTAVLRQW | VAPGTAVLRQW | VAPGTAVLRQ W | nsp16 | | 6877-6887 | 6.291399488 |
| B58 COVID 11 | APGTAVLRQW | APGTAVLRQW | APGTAVLRQW | APGTAVLRQW | nsp16 | | 6878-6887 | 6.418435098 |
| B58 COVID 12 | GVDIAANTVIW | GVDIAANTVIW | GVDIAANTVIW | GVDIAANTVIW | nsp15 | Middle domain | 6529-6539 | 8.025100372 |
| B58 COVID 13 | PGTAVLRQW | PGTAVLRQW | PGTAVLRQW | PGTAVLRQW | nsp16 | | 6879-6887 | 6.988429462 |
| B58 COVID 19 | KTSVDCTMY | KTSVDCTMY | KTSVDCTMY | KTSVDCTMY | spike | S2 domain | 733-741 | 4.349010357 |
| B81 COVID 6 | VVNAANVYL | VVNAANVYL | VVNAANVYL | VVNAANVYL | nsp3 | ADP Ribose Phosphatase | 1057-1065 | 4.083107174 |
| B81 COVID 11 | VIPTITQMNL | VIPTITQMNL | VIPTITQMNL | VIPTITQMNL | nsp12 | Polymerase, fingers domain | 4927-4936 | 5.848402913 |
| B81 COVID 14 | NPLLYDANYFL | NPLLYDANYFL | NPLLYDANYFL | NPLLYDANYFL | protein 3a | | 137-147 | 3.651957922 |
| CW07 COVID 11 | AMPNMLRIM | AMPNMLRIM | AMPNMLRIM | AMPNMLRIM | nsp12 | Polymerase, palm-fingers domain interface | 5018-5026 | 4.752547417 |
| CW07 COVID 16 | LRIAGHHL | LRIAGHHL | LRIAGHHL | LRIAGHHL | membrane | | 149-156 | 3.904974107 |

Table 6 depicts proteomic regions within the SARS-CoV-2 proteome that contain highly networked CTL epitopes derived from SARS-CoV-2 structural and accessory proteins. P

TABLE 6

Focused Epitope Regions

| Protein | Domain | Amino Acid Numbers | Amino Acid Sequence |
|---|---|---|---|
| Nucleocapsid | RNA-binding Domain | 77-87 | NSSPDDQIGYY |
| Nucleocapsid | Dimerization Domain | 275-317 | RRGPEQTQGNFGDQELIRQGTDYKHWPQIAQFAPSASAFFGM |
| Membrane | | 101-156 | RLFARTRSMWSFNPETNILLNVPLHGTILTRPLLESELVIGAVILRGHLRIAGHHL |
| ORF3a | | 137-167 | NPLLYDANYFLCWHTNCYDYCIPYNSVTSSI |
| Spike | S1 Domain | 34-47 | RGVYYPDKVFRSSV |
| Spike | RBD | 299-415 | TKCTLKSFTVEKGIYQTSNFRVQPTESIVRFPNITNLCPFGEVFNATRFASVYAWNRKRISNCVADYSV LYNSASFSTFKCYGVSPT KLNDLCFTNVYADSFVIRGDEVRQIAPGQT |
| Spike | S1 Domain | 534-639 | FELLHAPATVCGPKKSTNLVKNKCVNFNFNGLTGTGVLTESNKKFLPFQQFGRDIADTTDAVRDPQTL EILDITPCSFGGVSVITPGTNTSNQVAVLYQDVNCTEVPVAIHADQLTPTWRVYSTG |
| Spike | S2 Domain | 723-896 | TTEILPVSMTKTSVDCTMYICGDSTECSNLLLQYGSFCTQLNRALTGIAVEQDKNTQEVFAQVKQIYKT PPIKDFGGFNFSQILPDPSKPSKRSFIEDLLFNKVTLADAGFIKQYGDCLGDIAARDLICAQKFNGLTVLP PLLTDEMIAQYTSALLAGTITSGWTFGAGAALQI |
| Spike | HR1 | 923-1003 | IANQFNSAIGKIQDSLSSTASALGKLQDVVNQNAQALNTLVKQLSSNFGAISSVLNDILSRLDKVEAEVQI DRLITGRLQS |
| Spike | HR2 + S2 | 1038-1121 | KRVDFCGKGYHLMSFPQSAPHGVVFLHVTYVPAQEKNFTTAPAICHDGKAHFPREGVFVSNGTHWFV TQRNFYEPQIITTDNTF |

Table 7 depicts the foldable protein domains with the SARS-CoV-2 proteome that contain highly networked CTL epitopes derived from SARS-CoV-2 structural and accessory proteins.

TABLE 7

Foldable Domains

| Protein | Domain | Amino Acid Numbers | Amino Acid Sequence |
|---|---|---|---|
| Nucleocapsid | RNA-binding Domain | 50-173 | ASWFTALTQHGKEDLKFPRGQGVPINTNSSPDDQIGYYRRATRRIRGGDGKMKDLSPRWYFYYLGTGPEAGLP YGANKDGIIW VATEGALNTPKDHIGTRNPANNAAIVLQLPQGTTLPKGFYA |
| Nucleocapsid | Dimerization Domain | 257-364 | KPRQKRTATKAYNVTQAFGRRGPEQTQGNFGDQELIRQGTDYKHWPQIAQFAPSASAFFGMSRIGMEVTPSGT WLTYTGAIKL DDKDPNFKDQVILLNKHIDAYKTFP |
| Membrane | | 104-222 | ARTRSMWSFNPETNILLNVPLHGTILTRPLLESELVIGAVILRGHLRIAGHHLGRCDIKDLPKEITVATSRTLSYYKL GASQRVAGD SGFAAYSRYRIGNYKLNTDHSSSSDNIALLVQ |
| ORF3a | | 133-233 | CRSKNPLLYDANYFLCWHTNCYDYCIPYNSVTSSIVITSGDGTTSPISEHDYQIGGYTEKWESGVKDCVVLHSYF TSDYYQLYST QLSTDTGVEHVTFFIYNKIVDEPEEHVQIHTIDGSSGVVNPVMEPIYDEPTTTTSVPL |
| Spike | S1 Domain | 34-47 | RGVYYPDKVFRSSV |
| Spike | RBD/S1 Domains | 292-639 | ALDPLSETKCTLKSFTVEKGIYQTSNFRVQPTESIVRFPNITNLCPFGEVFNATRFASVYAWNRKRISNCVADYSV LYNSASFSTF KCYGVSPTKLNDLCFTNVYADSFVIRGDEVRQIAPGQTGKIADYNYKLPDDFTGCVIAWNSNNLDSKVGGNYNY LYRLFRKSNL KPFERDISTEIYQAGSTPCNGVEGFNCYFPLQSYGFQPTNGVGYQPYRVVVLSFELLHAPATVCGPKKSTNLVK NKCVNFNFN GLTGTGVLTESNKKFLPFQQFGRDIADTTDAVRDPQTLEILDITPCSFGGVSVITPGTNTSNQVAVLYQDVNCTEV PVAIHADQL TPTWRVYSTG |
| Spike | HR1/HR2/S2 Domains | 710-1147 | NSIAIPTNFTISVTTEILPVSMTKTSVDCTMYICGDSTECSNLLLQYGSFCTQLNRALTGIAVEQDKNTQEVFAQVK QIYKTPPIKDF GGFNFSQILPDPSKPSKRSFIEDLLFNKVTLADAGFIKQYGDCLGDIAARDLICAQKFNGLTVLPPLLTDEMIAQYT SALLAGTITSG WTFGAGAALQIPFAMQMAYRFNGIGVTQNVLYENQKLIANQFNSAIGKIQDSLSSTASALGKLQDVVNQNAQAL NTLVKQLSSNF GAISSVLNDILSRLDKVEAEVQIDRLITGRLQSLQTYVTQQLIRAAEIRASANLAATKMSECVLGQSKRVDFCGKG YHLMSFPQSAP HGVVFLHVTYVPAQEKNFTTAPAICHDGKAHFPREGVFVSNGTHWFVTQRNFYEPQIITTDNTFVSGNCDVVIGI VNNTVYDPLQP ELDS |

Alignment of highly stabilizing epitopes with bat CoV RaTG13, SARS-CoV-1, MERS-CoV and the common cold coronaviruses (HKU1, OC43, 229E, NL63) revealed that 65% of epitopes have ≤1 amino acid variants, and >90% of epitopes have ≤2 amino acid variants across the Sarbecovirus subgenus (bat CoV, SARS-CoV-1), but substantially higher levels of sequence mismatch for non-lineage B betacoronaviruses. This suggests that highly networked, HLA stabilizing SARS-CoV-2 epitopes have the potential to provide broad protection against circulating SARS-CoV-2 variants and CoVs across the Sarbecovirus subgenus.

Specific assessment of the 39 mutations in the SARS-CoV-2 VOCs revealed that only two amino acid mutations (Spike S982A in B.1.1.7, Nucleocapsid P80R in P.1) were found in the highly networked epitopes from structural and accessory proteins (Table 3), leading to exact sequence matching or <1 amino acid mutation for 100% of epitopes. The impact of these mutations on HLA class I-peptide stability was assessed and it was determined that there was no significant difference between parental sequence epitopes and the five mutated epitopes in the VOCs (FIG. 8G), indicating that highly networked CD8⁺ T cell epitopes would provide broad VOC coverage with maintained HLA class I presentation.

To determine whether highly networked epitopes had inherent mutational constraints in vivo that would confer broad protection against the emergence of viral escape variants, deep sequencing data of 747 primary SARS-CoV-2 isolates was utilized to reveal the mutational frequencies of 26 HLA-A*02-restricted CD8⁺ T cell epitopes (Agerer et al., 2021). Importantly, three of these epitopes were identified as highly networked (ALNTLVKQL, Spike 958-966; KLNDLCFTNV, Spike 386-395; VLNDILSRL, Spike 976-984). Given that each viral isolate was sequenced to a similar depth and the prevalence of the HLA-A*02 allele in the affected population was ~30%, it was determined that this was a highly relevant dataset to compare the in vivo viral evolution of highly networked and non-networked epitopes. The frequencies of networked and non-networked HLA-A*02 epitopes was compared with mutations at HLA anchor and TCR contact sites (position 2 through the terminal amino acid) that achieved an allelic frequency of 0.1 (i.e. tolerated mutations nearing fixation) and 0.9 (i.e. achieved mutational fixation) (Agerer et al., 2021). This revealed a striking difference with 6.67% (2/30) of networked epitope variants having an allelic frequency of 0.1 and 0% (0/30) having an allelic frequency of 0.9, whereas 25.2% (66/262) of non-networked epitope variants achieved an allelic frequency of 0.1 (P = 0.02) and 16.8% (44/262) achieved mutational fixation with an allelic frequency >0.9 (P = 0.01) (FIG. 8H). Alternatively, while the networked epitopes represented 10.3% of the analyzed epitope sequences, they accounted for only 2.9% of all epitope sequences with allelic frequencies >0.1 and 0% of epitope sequences with allelic frequencies >0.9. Given the broad targeting of epitopes by T cells in COVID-19 (Tarke et al., 2021), these analyses suggest that highly networked epitopes have significant constraints on in vivo viral evolution in comparison to non-networked epitopes restricted by the same HLA allele.

Collectively, the highly networked epitopes demonstrating HLA-peptide binding affinity for 18 HLA alleles include

AGEAANFCAL, ALNTLVKQL, AMPNMLRIM, APGTAVLRQW,
APSASAFF, APSASAFFGM, AQFAPSASA, AQVLSEMVM, ARTRSMWSF, AWPLIVTAL,
DRAMPNML, FCYMHHMEL, FELLHAPATV, FPQSAPHGV, FPQSAPHGVVF,
GEAANFCAL, GHLRIAGHHL, GNYQCGHYK, GTAVLRQW, GVDIAANTVIW,
GVFVSNGTHW, IAANTVIW, ILPVSMTK, IPTITQMNL, IPYNSVTSSI, IYQTSNFRV,
KGIYQTSNF, KGIYQTSNFR, KLNDLCFTNV, KLNDLCFTNVY, KQASLNGVTL,
KRNVIPTITQM, KRVDFCGK, KRVDFCGKGY, KTSVDCTMY, KWADNNCYL,
LLKSAYENF, LLTLQQIEL, LLYDANYFL, LPVSMTKTSV, LRIAGHHL, LRQWLPTGTL,
LRQWLPTGTLL, MIAQYTSAL, MPILTLTRAL, MVMCGGSLY, MVMCGGSLYV,
MWSFNPETNIL, NASSSEAFL, NPLLYDANYFL, NSSPDDQIGY, NSSPDDQIGYY,
NVIPTITQM, PDDQIGYY, PGTAVLRQW, PLLTDEMIAQY, QFAPSASAF, QFAPSASAFF,
QPGQTFSVL, QPTESIVRF, QTFSVLACY, QVNGLTSIKW, QWLPTGTLL,
RGVYYPDKVF, RLFARTRSMW, RQLLFVVEV, RQWLPTGTL, RQWLPTGTLL,
RRGPEQTQGNF, RTRSMWSF, RVIHFGAGSDK, RVQPTESIVRF, SALNHTKKW,
SEMVMCGGSL, SEYTGNYQC, SFNPETNIL, SFNPETNILL, SIKNFKSVL,
SIKWADNNCY, SMWSFNPET, SPDDQIGYY, SSPDDQIGYY, SSPDDQIGYY, TEILPVSM,
TILTRPLL, TSNEVAVLY, TTLPVNVAF, VAPGTAVLRQW, VIPTITQMNL, VLNDILSRL,
VMCGGSLYV, VMCGGSLYVK, VNGLTSIKW, VPVVDSYY, VSMTKTSV,
VTANVNALL, VVNAANVYL, YDANYFLCW, YHLMSFPQSA, YLATALLTL,
YPKCDRAM, YQCGHYKHI, YQDVNCTEV, YRFNGIGV, YTGNYQCGHY,
YYPDKVFRSSV, YYSLLMPIL and/or YYSLLMPILTL

(FIG. 9). Additional highly networked epitopes that bound to the other HLA alleles with high affinity and prolonged stabilization were selected for further T cell vaccine development (FIG. 9).

### Example 6

### Convalescent COVID-19 Patients Exhibit CD8+ T Cell Reactivity to Highly Networked Epitopes

To evaluate the immunogenicity of highly networked, HLA stabilizing SARS-CoV-2 epitopes, the reactivity of CD8+ T cells within a cohort of 20 healthy donors (HDs) and 30 convalescent COVID-19 patients (Table 4) was assessed.

TABLE 4

Characteristics of healthy donors and convalescent COVID-19 patients utilized for IFN-gamma ELISpot assays

| | Unexposed (n = 20) | COVID-19 (n = 30) |
|---|---|---|
| Age (years) | 23-63 (median = 30, IQR = 16.5) | 20-63 (median = 36, IQR = 22.5) |
| Gender | | |
| Male (%) | 25% (5/20) | 23.3% (7/30) |
| Female (%) | 75% (15/20) | 76.6% (23/30) |
| Sample Collection Date (Range) | January 2015-January 2020 | April 2020-August 2020 |
| Disease Severity | | |
| Mild (%) | N/A | 70% (21/30) |
| Moderate (%) | N/A | 20% (6/30) |
| Severe (%) | N/A | 10% (3/30) |
| Symptoms | | |
| Cough | N/A | 43.3% (13/30) |
| Fever | N/A | 40% (12/30) |
| Anosmia | N/A | 23.3% (7/30) |
| Dyspnea | N/A | 23.3% (7/30) |
| Diarrhea | N/A | 0.07% (2/30) |
| Mylagias | N/A | 36.7% (11/30) |
| Days Post-Symptom Resolution at Collection | N/A | 7-92 (Median = 30.5, IQR = 24.25) |
| Hypertension | N/A | 16.7% (5/30) |
| Hyperlipidemia | N/A | 0.07% (2/30) |
| Diabetes | N/A | 0.07% (2/30) |
| Asthma | N/A | 0.03% (1/30) |

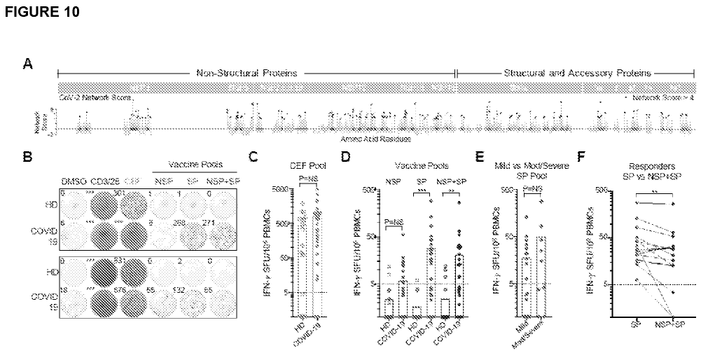

CD4-depleted peripheral blood mononuclear cells (PBMCs) were tested for responses to peptide pools of highly networked epitopes derived from non-structural proteins (NSP), structural proteins (SP) or a combination of non-structural and structural proteins (NSP+SP) (FIG. 10A) using ex vivo interferon-γ (IFN-γ) enzyme-linked immunospot (ELISpot) assays (FIG. 10B). Anti-CD3/CD28 antibodies and a pool of CMV, EBV and Flu (CEF) peptides were used as positive controls, while DMSO was used as a negative control. Importantly, CEF-specific CD8⁺ T cell responses were not significantly different between the two patient groups (FIG. 10C). However, significant differences were observed in IFN-γ⁺ CD8⁺ T cell responses to highly networked, HLA stabilizing epitopes in the SP peptide pool (1/20 HDs vs 15/30 COVID-19; P = 0.0003) and combined NSP+SP pool (3/20 HDs vs. 13/30 COVID-19; P = 0.001) but not the NSP pool alone (2/20 HDs vs. 8/20 COVID-19; P = 0.2627) (FIG. 10D). This is consistent with prior reports that observed stronger SARS-CoV-2-specific CD8⁺ T cell responses to epitopes derived from the higher abundance structural proteins than to epitopes from non-structural proteins (Grifoni et al., 2020b; Le Bert et al., 2020). In addition, similar to a prior study (Peng et al., 2020), a higher average magnitude of IFN-γ CD8⁺ T cell response to the SP pool occurred in convalescent COVID-19 patients with moderate-to-severe disease (n = 9) than in those with mild disease (n = 21) (FIG. 10E), although this did not reach statistical significance (P = 0.2696). Interestingly, in patients who responded to the highly networked SP peptide pool, a significant decrease in CD8⁺ T cell reactivity of individual participants was observed when incubated with the combination SP+NSP peptide pool (13/15 individuals) (FIG. 10F). These data suggest that incorporating HLA stabilizing epitopes from non-structural proteins in a vaccine immunogen could negatively affect subsequent recognition of structural and accessory protein epitopes by CD8+ T cells. Importantly though, these data confirm the immunogenicity of highly networked, HLA stabilizing epitopes derived from structural and accessory proteins, implicating their potential utility as candidates for a SARS-CoV-2 T cell-based vaccine.

### Example 7

### Generation of a T Cell Response in Mice Against Highly Networked SARS-CoV-2 Epitopes

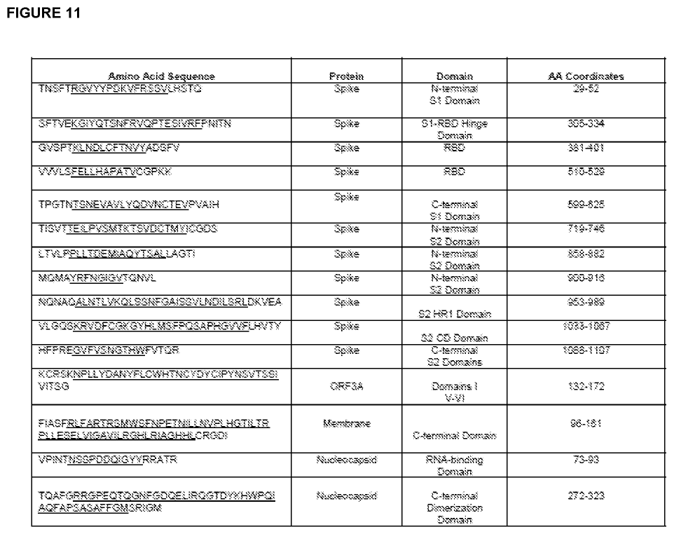

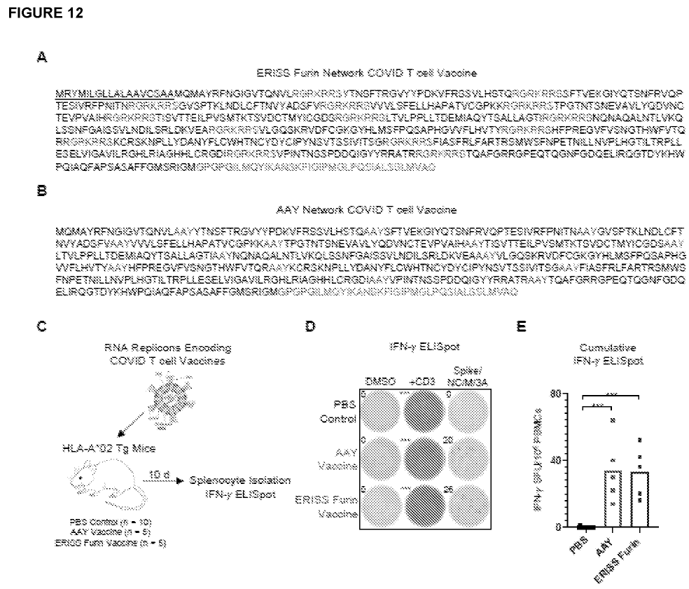

The immunogen is made up of the 15 regions in SARS-CoV-2 structural and accessory proteins shown in FIG. 11 in two cassette designs. The first cassette (ERISS Furin Network COVID T cell vaccine) has an N-terminal endoplasmic reticulum insertion signal sequence (MRYMILGLLALAAVCSAA; underlined), a furin cleavage sequence (RGRKRRS; red) between each highly networked SARS-CoV-2 sequence depicted in FIG. 11 and a C-terminal universal tetanus and diphtheria toxoid CD4+ T cell helper epitope (TpD; green) preceded by a GPGPG linker (blue) (FIG. 12A). The second cassette (AAY Network COVID T cell vaccine) has each highly networked SARS-CoV-2 sequence in FIG. 11 linked by an Alanine-Alanine-Tyrosine (AAY) sequence (red) and a C-terminal universal tetanus and diphtheria toxoid CD4+ T cell helper epitope (TpD; green) preceded by a GPGPG linker (blue) (FIG. 12B). These cassettes were encoded into an alphavirus-based RNA replicon, encapsulated with lipid nanoparticles and delivered to HLA-A*02 transgenic mice by intra-muscular injection (B6.Cg-Immp2l^{Tg(HLA-A/H2-D)2Enge}; Jackson Laboratories) (FIG. 12C), with mice receiving either PBS control infector (n=10), ERISS Furin Network COVID T cell vaccine replicon injection (n=5) or AAY Network COVID T cell vaccine replicon injection (n=5). The induction of de novo T cell responses was determined by assessment of IFN-γ+ T cells by ELISpot in mice vaccinated with the networked COVID T cell immunogens 10 days after vaccination, in response to overlapping peptide pools of structural and accessory SARS-CoV-2 proteins. Briefly, mouse splenocytes were harvested and 5×10⁵ cells were incubated overnight with either no peptide DMSO control, a positive control (anti-mouse CD3 antibody; clone 17A2; BioLegend) or a combined overlapping peptide pool of Spike, Nucleocapsid, ORF3A and Membrane proteins (JPT Peptide Technologies; lug/mL for each overlapping peptide) in duplicate. Representative IFN-γ ELISpot plots demonstrating the successful induction of IFN-γ+ T cell responses in vaccinated animals to the SARS-CoV-2 structural and accessory protein overlapping peptide pools are depicted in FIG. 12D. The number of IFN-γ spot forming units (SFUs) is listed in the upper left of each well. A value of *** indicates that the response exceeded assay detection limits. A comparison of the number of IFN-γ SFUs per 1×10⁶ splenocytes between control and vaccinated animals reveals a significant difference in the magnitude of SARS-CoV-2 specific T cell responses (FIG. 12E). Statistical comparisons were made using Mann-Whitney U test. Calculated P values were as follows: *P < 0.05; **P < 0.01; ***P < 0.001; ****P < 0.0001.

### Experimental Design for Examples 3-6

Cell lines: HEK293T cells used for lentivirus production and ACE2-expressing HEK293T cells (a gift from A. Balazs) used for lentivirus infection were maintained in advanced DMEM (Sigma-Aldrich) supplemented with 10% FBS, 1X Penicillin-Streptomycin-L-Glutamine mixture (Gibco), 1X non-essential amino acids (Gibco), 1X sodium pyruvate (Gibco), and 1X HEPES buffer (Corning) (D10). The human B cell lines 721.221 were generated previously by γ-radiation of 721 cells and do not express HLA A and B alleles (Shimizu and DeMars, 1989). These cell lines were maintained in RPMI-1640 medium (Sigma-Aldrich) supplemented with 10% (v/v) FBS (Sigma-Aldrich) and 1X Penicillin-Streptomycin-L-Glutamine mixture (Gibco). TAP-deficient mono-allelic HLA class I-expressing 721.221 cells were generated as described previously (please see companion manuscript) and maintained in 5ug/mL blasticidin (Invivogen), 0.5 ug/ml puromycin (Invivogen) and 1.5 mg/ml G418 (Invivogen).

Human Subjects: Peripheral blood mononuclear cells (PBMCs) were isolated from healthy human volunteers or SARS-CoV-2 infected patients by Ficoll gradient separation from ACD tubes. They were then cryopreserved and stored in liquid nitrogen prior to experimental use. The study was approved by the MGH Institutional Review Board. All subjects were between 18-65 years of age, provided informed consent and were confirmed to have a test positive for SARS-CoV-2 using PCR with reverse transcription from an upper respiratory tract (nose and throat) swab tested at an accredited laboratory. The degree of disease severity was identified as mild, severe or critical infection, according to recommendations from the World Health Organization. Patients were classified as having mild symptoms if they did not require oxygen (that is, their oxygen saturation was 94% or greater on ambient air) and if their symptoms were managed at home. Moderate-to-severe infection was defined as one of the following conditions in a patient confirmed as having COVID-19: respiratory distress with a respiratory rate of >30 breaths per minute; blood oxygen saturation of <94%; or arterial oxygen partial pressure/FiO2 < 300 mmHg. SARS-CoV-2 protein structures: For the analysis of the SARS-CoV-2 proteome, the following PDB files were utilized: NSP3 ADP ribose phosphatase domain (PDB: 6W02), NSP3 papain-like protease (PDB: 6W9C), NSP5 3CL protease (PDB: 6YB7), NSP7 (PDB: 6M7I, Chain C). NSP8 (PDB: 6M7I, Chain B, D), NSP9 (PDB: 6W4B), NSP10 (6W4H, Chain B), NSP12 RNA-dependent RNA polymerase (6M7I, Chain A), NSP15 (PDB: 6W01), NSP16 (PDB: 6W4H, Chain A), Spike closed conformation (PDB: 6VXX), Nucleocapsid RNA-binding domain (PDB: 6VYO), Nucleocapsid dimerization domain (PDB: 6WJI), ORF3a (PDB: 6XDC), ORF7a (PDB: 6W37), Spike open conformation (PDB: 6VYB), and Spike receptor binding domain (PDB: 6M0J). The membrane structure was downloaded from from DeepMind (https://deepmind.com/research/open-source/computational-predictions-of-protein-structures-associated-with-COVID-19) on Apr. 8, 2020. MODELLER (https://salilab.org/modeller/) was used to create homology models for the envelope protein using SARS-CoV-1 envelope (PDB: 5×29) as a template. Water molecules and solvents were removed from each PDB file prior to analysis.

SARS-CoV-1 protein structures: For the analysis of the SARS-CoV-1 proteome, the following PDB files were utilized: NSP3 ADP ribose phosphatase domain (PDB: 2FAV), NSP3 papain-like protease (PDB: 5Y3Q), NSP5 3CL protease (PDB: 1Q2W), NSP7 (PDB: 6NUR, Chain C). NSP8 (PDB: 6NUR, Chain B, D), NSP9 (PDB: 1QZ8), NSP10 (2XYQ, Chain B), NSP12 RNA-dependent RNA polymerase (6NUR, Chain A), NSP15 (PDB: 2H85), NSP16 (PDB: 2XYQ, Chain A), Spike (PDB: 5XLR), Nucleocapsid RNA-binding domain (PDB: 1SSK), and Nucleocapsid dimerization domain (PDB: 2GIB). Water molecules and solvents were removed from each PDB file prior to analysis.

MERS-CoV protein structures: For the analysis of the MERS proteome, the following PDB files were utilized: NSP3 ADP ribose phosphatase domain (PDB: 5HOL), NSP3 papain-like protease (PDB: 4RNA), NSP5 3CL protease (PDB: 4WME), NSP10 (5YN5, Chain B), NSP15 (PDB: 5YVD), NSP16 (PDB: 5YN5, Chain A), Spike (PDB: 5×59), Nucleocapsid RNA-binding domain (PDB: 4UD1), and Nucleocapsid dimerization domain (PDB: 6G13). Water molecules and solvents were removed from each PDB file prior to analysis.

Reference genomes: For the analysis of the highly stabilizing epitopes across human coronaviruses, the following reference genomes were utilized: bat coronavirus RaTG13 (GenBank: MN996532.1), SARS-CoV1 (GenBank: AY274119.3), MERS (GenBank: JX869059.2), HCoV-OC43 (GenBank: AY391777.1), HCoV-HKU1 (GenBank: AY884001.1), HCoV-229E (GenBank: KY684760.1), and HCoV-NL63 (NCBI Reference Sequence: NC_005831.2).

Shannon entropy and conservation scoring: Between 617-1213 MERS and 219-725 sarbecovirus (SARS-CoV-1/Bat) sequences, and between 55031-110163 SARS-CoV-2 protein sequences were downloaded from NCBI. MERS and sarbecovirus sequences were downloaded on May 18, 2020 and SARS-CoV-2 sequences on Feb. 7, 2020. Using the protein sequence derived from SARS-CoV-2 PDB structures as a reference in each protein sequence alignment, amino acid frequencies at each amino acid position were tabulated. Shannon entropy,H(p), was calculated based on the following formula (Lund et al., 2005): H(p) = -Σₐpₐ log₂(pₐ) where pₐ is the proportion of amino acid a at a given position and qₐ is the background frequency of amino acid a.

Generation of SARS-CoV-2 Spike mutants: HDM-SARS2-Spike-delta21 was a gift from Jesse Bloom (Addgene plasmid # 155130; http://n2t.net/addgene:155130; RRID: Addgene_155130) and was modified to express one of several individual mutations using the Q5 Site-Directed Mutagenesis Kit (New England Biolabs) according to the manufacturer’s instructions. Back-to-back 5′ oligonucleotide primers were utilized to engineer individual mutants (Table 1) within the HDM-SARS2-Spike-delta21 plasmid. Confirmation of successful mutagenesis was accomplished by complete plasmid sequencing (MGH Sequencing Core). Full-length viral plasmids were propagated in Stellar competent cells (Takara Bio) and DNA plasmid stocks were prepared using a QiaPrep spin miniprep kit (Qiagen).

Generation of SARS-CoV-2 Spike Pseudotyped Lentivirus: SARS-CoV-2 Spike pseudotyped lentivirus was produced as previously described (Crawford et al., 2020). Briefly, HEK293T cells were transfected with 1 µg pHAGE-CMV-Luc2-IRES-ZsGreen-W (BEI), a lentiviral backbone plasmid expressing luciferase under a CMV promoter and an IRES followed by ZsGreen, 0.22 µg HDM-Hgpm2 (BEI), a lentiviral helper plasmid expressing HIV Gag-Pol under a CMV promoter, 0.22 µg HDM-tat1b (BEI), a lentiviral helper plasmid expressing HIV Tat under a CMV promoter, 0.22 µg pRC-CMV-Revlb (BEI), a lentiviral helper plasmid expressing HIV Rev under a CMV promoter, and 0.34 µg of the plasmid encoding HDM-SARS2-Spike-delta21 using polyethylenimine (Polyplus) in serum-free Dulbecco’s Modified Eagle’s Medium (Sigma-Aldrich) supplemented with 25 mM HEPES buffer (Corning). Media was changed to D10 24 h post-transfection. After 48 h, pseudotyped lentivirus was harvested by filtering supernatant through a 0.45 µm low protein binding durapore membrane (Millipore). Frozen aliquots were stored at -80° C. and viral concentrations were quantified using the colorimetric Reverse Transcriptase Assay (Sigma-Aldrich). All packaging plasmids were propagated in DH5α cells (NEB).

SARS-CoV-2 Spike Pseudotyped Lentiviral infectivity assay: HEK293T and ACE2-expressing HEK293T cells were seeded at a density of 1.25×10⁴ cells/well into a 96-well plate one day prior to infection with 60 µL wild-type or mutant Spike pseudotyped lentivirus diluted two-fold in D10 with 5 µg/mL Polybrene Transfection Reagent (Millipore). 24h following infection, an additional 140 µL of D10 was added and cells were cultured at 37° C. and 5% CO2 for 48 h. Cells were harvested, stained with viability dye, fixed in 2% paraformaldehyde and subsequently analyzed for ZsGreen expression via flow cytometry using a BD LSR II (BD Biosciences). Flow cytometric data were analyzed using FlowJo software (v10.1r5).

Peptide synthesis reagents: Fmoc-protected amino acids and synthesis resin, 2-Chlorotrityl chloride were purchased from Akaal Organics (Long Beach, CA). Dimethylformamide (DMF), N-methyl pyrrolidone (NMP), Acetonitrile and Methyl-tert. Butyl Ether (MTBE) were purchased from Fisher Bioreagents (Fair Lawn, NJ). 2-(6-Chloro-1-H-benzotriazole-1-yl)-1,1,3,3-tetramethylaminium hexafluorophosphate (HCTU) was purchased from AAPPTEC (Louisville, KY). Piperidine and Dichloromethane (DCM) were from EMD-Millipore (Billerica, MA). Diisopropylethylamine (DIEA), N-Methyl-morpholine (NMM), Triisoprpopyl-silane, 3,6-dioxa-1,8-octanedithiol (DODT) and trifluoroacetic acid (TFA) were purchased from Sigma-Aldrich. Peptide synthesis and analysis: Peptides were synthesized on an automated robotic peptide synthesizer (AAPPTEC, Model 396 Omega) by using Fmoc solid-phase chemistry (Behrendt et al., 2016) on 2-chlorotrityl chloride resin (Chatzi et al., 1991). The C-terminal amino acids were loaded using the respective Fmoc-Amino Acids in the presence of DIEA. Unreacted sites on the resin were blocked using methanol, DIEA and DCM (15:5:80 v/v). Subsequent amino acids were coupled using optimized (to generate peptides containing more than 90% of the desired full-length peptides) cycles consisting of Fmoc removal (deprotection) with 25% Piperidine in NMP followed by coupling of Fmoc-AAs using HCTU/NMM activation. Each deprotection or coupling was followed by several washes of the resin with DMF to remove excess reagents. After the peptides were assembled and the final Fmoc group removed, peptide resin was then washed with dimethylformamide, dichloromethane, and methanol three times each and air dried. Peptides were cleaved from the solid support and deprotected using odor free cocktail (TFA/triisopropyl silane/water/DODT; 94/2.5/2.5/1.0 v/v) for 2.5 h at room temperature (Teixeira et al., 2002). Peptides were precipitated using cold methyl tertiary butyl ether (MTBE). The precipitate was washed 2 times in MTBE, dissolved in a solvent (0.1% trifluoroacetic acid in 30%Acetonitrile/70%water) followed by freeze drying. Peptides were characterized by Ultra Performance Liquid Chromatography (UPLC) and Matrix Assisted Laser Desorption/Ionization Mass Spectrometry (MALDI-MS). All peptides were dissolved initially in 100% DMSO at a concentration of 40 mM, prior to dilution at the appropriate concentration in RPMI-1640 medium.

Antibodies and flow cytometry: Flow cytometric analyses were performed using HLA-ABC (W6/32) APC (1:100; Biolegend)(Parham et al., 1979) and LIVE/DEAD violet viability dye (1:1000; Life Technologies). Cell surface staining of HLA expression was performed on cells grown in 96-well plates in 200 µL volume. Cells were stained with antibody and viability dye in PBS + 2% FBS for 20 min at 4° C. and fixed in 4% paraformaldehyde, prior to flow cytometric analysis using a BD LSR II (BD Biosciences). Flow cytometric data were analyzed using FlowJo software (v10.1r5; Treestar).

HLA class I-peptide concentration-based stability assay: For concentration-based HLA class I-peptide stability binding assays, 5×10⁴ TAP-deficient mono-allelic HLA class I expressing 721.221 cells were incubated with peptides in concentrations ranging from 0.1 to 100 µM, and 3 µg/mL of β2m (Sino Biological, Wayne, PA, USA), in RPMI-1640 medium overnight at 26° C./5% CO2 for 18 hours. Controls without peptide, but the corresponding concentration of DMSO, were performed in parallel. Following overnight incubation, cells were incubated at 37° C./5% CO2 prior to staining for viability and HLA class I surface expression with HLA-ABC APC antibody (1:100), and subsequent analysis by flow cytometry.

Ex vivo ELISpot assay: IFN-γ ELISpot assays were performed according to the manufacturer’s instructions (Mabtech). PBMCs were first depleted of CD4⁺ T cells by CD4 depletion kit (Miltenyi Biotec). 500,000 CD4-depleted PBMCs per test were then incubated with SARS-CoV-2 peptide pools at a final concentration of 1 µg/ ml for 16-18 h. CEF peptide pool (Mabtech; lug/mL), anti-CD3 (Clone OKT3, Biolegend, lug/mL) and anti-CD28 Ab (Clone CD28.2, Biolegend, lug/mL) were used as positive controls. To quantify antigen-specific responses, mean spots of the DMSO control wells were subtracted from the positive wells, and the results were expressed as spot-forming units (SFU) per 10⁶ PBMCs. Responses were considered positive if the results were >5 SFU/10⁶ PBMCs following control subtraction. If negative DMSO control wells had >30 SFU/10⁶ PBMCs or if positive control wells (anti-CD3/anti-CD28 stimulation) were negative, the results were excluded from further analysis.

The Examples are put forth for illustrative purposes only and are not intended to limit the scope of what the inventors regard as their invention.

All references cited herein, including patents, patent applications, papers, text books, and the like, and the references cited therein, to the extent that they are not already, are hereby incorporated herein by reference in their entirety. Although the forgoing invention has been described in some detail by way of illustration and example for clarity and understanding, it will be readily apparent to one ordinary skill in the art in light of the teachings of this invention that certain variations, changes, modifications and substitution of equivalents may be made thereto without necessarily departing from the spirit and scope of this invention. As a result, the implementations described herein are subject to various modifications, changes and the like, with the scope of this invention being determined solely by reference to the claims appended hereto. Those of skill in the art will readily recognize a variety of non-critical parameters that could be changed, altered or modified to yield essentially similar results.

### REFERENCES

Agerer, B., Koblischke, M., Gudipati, V., Montano-Gutierrez, L.F., Smyth, M., Popa, A., Genger, J.-W., Endler, L., Florian, D.M., Mühlgrabner, V., et al. (2021). SARS-CoV-2 mutations in MHC-I-restricted epitopes evade CD8+ T cell responses. Sci Immunol 6.

Ahmed, S.F., Quadeer, A.A., and McKay, M.R. (2020). Preliminary Identification of Potential Vaccine Targets for the COVID-19 Coronavirus (SARS-CoV-2) Based on SARS-CoV Immunological Studies. Viruses 12.

Baum, A., Fulton, B.O., Wloga, E., Copin, R., Pascal, K.E., Russo, V., Giordano, S., Lanza, K., Negron, N., Ni, M., et al. (2020). Antibody cocktail to SARS-CoV-2 spike protein prevents rapid mutational escape seen with individual antibodies. Science 369, 1014-1018.

Behrendt, R., White, P., and Offer, J. (2016). Advances in Fmoc solid-phase peptide synthesis. J. Pept. Sci. 22, 4-27.

Calis, J.J.A, de Boer, R.J., and Keşmir, C. (2012). Degenerate T-cell recognition of peptides on MHC molecules creates large holes in the T-cell repertoire. PLoS Comput. Biol. 8, e1002412.

Channappanavar, R., Fett, C., Zhao, J., Meyerholz, D.K., and Perlman, S. (2014). Virus-specific memory CD8 T cells provide substantial protection from lethal severe acute respiratory syndrome coronavirus infection. J. Virol. 88, 11034-11044.

Chatzi, K.B.O., Gatos, D., and Stavropoulos, G. (1991). 2-Chlorotrityl chloride resin: Studies on anchoring of Fmoc-amino acids and peptide cleavage. Int. J. Pept. Protein Res. 37, 513-520.

Crawford, K.H.D., Eguia, R., Dingens, A.S., Loes, A.N., Malone, K.D., Wolf, C.R., Chu, H.Y., Tortorici, M.A., Veesler, D., Murphy, M., et al. (2020). Protocol and Reagents for Pseudotyping Lentiviral Particles with SARS-CoV-2 Spike Protein for Neutralization Assays. Viruses 12.

Ferretti, A.P., Kula, T., Wang, Y., Nguyen, D.M.V., Weinheimer, A., Dunlap, G.S., Xu, Q., Nabilsi, N., Perullo, C.R., Cristofaro, A.W., et al. (2020). COVID-19 Patients Form Memory CD8+ T Cells that Recognize a Small Set of Shared Immunodominant Epitopes in SARS-CoV-2.

Finkel, Y., Mizrahi, O., Nachshon, A., Weingarten-Gabbay, S., Morgenstern, D., Yahalom-Ronen, Y., Tamir, H., Achdout, H., Stein, D., Israeli, O., et al. (2020). The coding capacity of SARS-CoV-2. Nature.

Folegatti, P.M., Ewer, K.J., Aley, P.K., Angus, B., Becker, S., Belij-Rammerstorfer, S., Bellamy, D., Bibi, S., Bittaye, M., Clutterbuck, E.A., et al. (2020). Safety and immunogenicity of the ChAdOxl nCoV-19 vaccine against SARS-CoV-2: a preliminary report of a phase ½, single-blind, randomised controlled trial. Lancet 396, 467-478.

Gaiha, G.D., Rossin, E.J., Urbach, J., Landeros, C., Collins, D.R., Nwonu, C., Muzhingi, I., Anahtar, M.N., Waring, O.M., Piechocka-Trocha, A., et al. (2019). Structural topology defines protective CD8+ T cell epitopes in the HIV proteome. Science 364, 480-484.

Gao, A., Chen, Z., Segal, F.P., Carrington, M., Streeck, H., Chakraborty, A.K., and Julg, B. (2020a). Predicting the Immunogenicity of T cell epitopes: From HIV to SARS-CoV-2. BioRxiv.

Gao, Q., Bao, L., Mao, H., Wang, L., Xu, K., Yang, M., Li, Y., Zhu, L., Wang, N., Lv, Z., et al. (2020b). Development of an inactivated vaccine candidate for SARS-CoV-2. Science 369, 77-81.

Greaney, A.J., Starr, T.N., Gilchuk, P., Zost, S.J., and Binshtein, E. (2020). Complete mapping of mutations to the SARS-CoV-2 spike receptor-binding domain that escape antibody recognition. BioRxiv.

Grifoni, A., Sidney, J., Zhang, Y., Scheuermann, R.H., Peters, B., and Sette, A. (2020a). A Sequence Homology and Bioinformatic Approach Can Predict Candidate Targets for Immune Responses to SARS-CoV-2. Cell Host Microbe 27, 671-680.e2.

Grifoni, A., Weiskopf, D., Ramirez, S.I., Mateus, J., Dan, J.M., Moderbacher, C.R., Rawlings, S.A., Sutherland, A., Premkumar, L., Jadi, R.S., et al. (2020b). Targets of T Cell Responses to SARS-CoV-2 Coronavirus in Humans with COVID-19 Disease and Unexposed Individuals. Cell 181, 1489-1501.e15.

Gur, M., Taka, E., Yilmaz, S.Z., Kilinc, C., Aktas, U., and Golcuk, M. (2020). Exploring Conformational Transition of 2019 Novel Coronavirus Spike Glycoprotein Between Its Closed and Open States Using Molecular Dynamics Simulations.

Harndahl, M., Rasmussen, M., Roder, G., Dalgaard Pedersen, I., Sørensen, M., Nielsen, M., and Buus, S. (2012). Peptide-MHC class I stability is a better predictor than peptide affinity of CTL immunogenicity. Eur. J. Immunol. 42, 1405-1416.

Jackson, L.A., Anderson, E.J., Rouphael, N.G., Roberts, P.C., Makhene, M., Coler, R.N., McCullough, M.P., Chappell, J.D., Denison, M.R., Stevens, L.J., et al. (2020). An mRNA vaccine against SARS-CoV-2-preliminary report. N. Engl. J. Med.

Keech, C., Albert, G., Cho, I., Robertson, A., Reed, P., Neal, S., Plested, J.S., Zhu, M., Cloney-Clark, S., Zhou, H., et al. (2020). Phase 1-2 Trial of a SARS-CoV-2 Recombinant Spike Protein Nanoparticle Vaccine. N. Engl. J. Med.

Le Bert, N., Tan, A.T., Kunasegaran, K., Tham, C.Y.L., Hafezi, M., Chia, A., Chng, M.H.Y., Lin, M., Tan, N., Linster, M., et al. (2020). SARS-CoV-2-specific T cell immunity in cases of COVID-19 and SARS, and uninfected controls. Nature 584, 457-462.

Letvin, N.L., Haynes, B.F., Hahn, B.H., and Korber, B. (2009). Expanded breadth of the T-cell response to mosaic human immunodeficiency virus type 1 envelope DNA vaccination. Journal Of.

Li, Q., Wu, J., Nie, J., Zhang, L., Hao, H., Liu, S., Zhao, C., Zhang, Q., Liu, H., Nie, L., et al. (2020). The Impact of Mutations in SARS-CoV-2 Spike on Viral Infectivity and Antigenicity. Cell 182, 1284-1294.e9.

Liao, M., Liu, Y., Yuan, J., Wen, Y., Xu, G., Zhao, J., and Cheng, L. (2020). Single-cell landscape of bronchoalveolar immune cells in patients with COVID-19. Nat. Med.

Liu, G., Carter, B., Bricken, T., Jain, S., Viard, M., Carrington, M., and Gifford, D.K. (2020a). Computationally Optimized SARS-CoV-2 MHC Class I and II Vaccine Formulations Predicted to Target Human Haplotype Distributions. Cell Syst 11, 131-144.e6.

Liu, J., Li, S., Liu, J., Liang, B., Wang, X., Wang, H., Li, W., Tong, Q., Yi, J., Zhao, L., et al. (2020b). Longitudinal characteristics of lymphocyte responses and cytokine profiles in the peripheral blood of SARS-CoV-2 infected patients. EBioMedicine 55, 102763.

Lund, O., Nielsen, M., Brunak, S., Lundegaard, C., and Kesmir, C. (2005). Immunological Bioinformatics (MIT Press).

Marsh, S.G.E., Parham, P., and Barber, L.D. (1999). The HLA FactsBook (Elsevier).

Meirson, T., Bomze, D., and Markel, G. (2020). Structural basis of SARS-CoV-2 spike protein induced by ACE2. Bioinformatics.

Menachery, V.D., Yount, B.L., Jr, Debbink, K., Agnihothram, S., Gralinski, L.E., Plante, J.A., Graham, R.L., Scobey, T., Ge, X.-Y., Donaldson, E.F., et al. (2016a). Corrigendum: A SARS-like cluster of circulating bat coronaviruses shows potential for human emergence. Nat. Med. 22, 446.

Menachery, V.D., Yount, B.L., Jr, Sims, A.C., Debbink, K., Agnihothram, S.S., Gralinski, L.E., Graham, R.L., Scobey, T., Plante, J.A., Royal, S.R., et al. (2016b). SARS-like WIV1-CoV poised for human emergence. Proc. Natl. Acad. Sci. U. S. A. 113, 3048-3053.

Mercado, N.B., Zahn, R., Wegmann, F., Loos, C., Chandrashekar, A., Yu, J., Liu, J., Peter, L., McMahan, K., Tostanoski, L.H., et al. (2020). Single-shot Ad26 vaccine protects against SARS-CoV-2 in rhesus macaques. Nature.

Mulligan, M.J., Lyke, K.E., Kitchin, N., Absalon, J., Gurtman, A., Lockhart, S., Neuzil, K., Raabe, V., Bailey, R., Swanson, K.A., et al. (2020). Phase ½ study of COVID-19 RNA vaccine BNT162b1 in adults. Nature.

Ng, O.-W., Chia, A., Tan, A.T., Jadi, R.S., Leong, H.N., Bertoletti, A., and Tan, Y.-J. (2016). Memory T cell responses targeting the SARS coronavirus persist up to 11 years post-infection. Vaccine 34, 2008-2014.

Parham, P., Barnstable, C.J., and Bodmer, W.F. (1979). Use of a monoclonal antibody (W6/32) in structural studies of HLA-A, B, C antigens. The Journal of Immunology 123, 342-349.

Peng, Y., Mentzer, A.J., Liu, G., Yao, X., Yin, Z., Dong, D., Dejnirattisai, W., Rostron, T., Supasa, P., Liu, C., et al. (2020). Broad and strong memory CD4+ and CD8+ T cells induced by SARS-CoV-2 in UK convalescent individuals following COVID-19. Nat. Immunol.

Poran, A., Harjanto, D., Malloy, M., Rooney, M.S., Srinivasan, L., and Gaynor, R.B. (2020). Sequence-based prediction of vaccine targets for inducing T cell responses to SARS-CoV-2 utilizing the bioinformatics predictor RECON.

Rasmussen, M., Fenoy, E., Harndahl, M., Kristensen, A.B., Nielsen, I.K., Nielsen, M., and Buus, S. (2016). Pan-Specific Prediction of Peptide-MHC Class I Complex Stability, a Correlate of T Cell Immunogenicity. The Journal of Immunology 197, 1517-1524.

Santra, S., Liao, H.-X., Zhang, R., Muldoon, M., Watson, S., Fischer, W., Theiler, J., Szinger, J., Balachandran, H., Buzby, A., et al. (2010). Mosaic vaccines elicit CD8+ T lymphocyte responses that confer enhanced immune coverage of diverse HIV strains in monkeys. Nat. Med. 16, 324-328.

Screaton, G.R., Hou, J., and McMichael, A.J. (2008). T cell responses to whole SARS coronavirus in humans. Of Immunology.

Sekine, T., Perez-Potti, A., Rivera-Ballesteros, O., Strålin, K., Gorin, J.-B., Olsson, A., Llewellyn-Lacey, S., Kamal, H., Bogdanovic, G., Muschiol, S., et al. (2020). Robust T cell immunity in convalescent individuals with asymptomatic or mild COVID-19. Cell.

Sette, A., and Sidney, J. (1999). Nine major HLA class I supertypes account for the vast preponderance of HLA-A and -B polymorphism. Immunogenetics 50, 201-212.

Shimizu, Y., and DeMars, R. (1989). Production of human cells expressing individual transferred HLA-A,-B,-C genes using an HLA-A,-B,-C null human cell line. J. Immunol. 142, 3320-3328.

Sidney, J., Peters, B., Frahm, N., Brander, C., and Sette, A. (2008). HLA class I supertypes: a revised and updated classification. BMC Immunol. 9, 1.

Soresina, A., Moratto, D., and Chiarini, M. (2020). Two X-linked agammaglobulinemia patients develop pneumonia as COVID-19 manifestation but recover. Pediatr. Allergy Immunol.

Starr, T.N., Greaney, A.J., Hilton, S.K., Crawford, K.H.D., Navarro, M.J., Bowen, J.E., Tortorici, M.A., Walls, A.C., Veesler, D., and Bloom, J.D. (2020). Deep mutational scanning of SARS-CoV-2 receptor binding domain reveals constraints on folding and ACE2 binding. BioRxiv.

Streeck, H., Jolin, J.S., Qi, Y., Yassine-Diab, B., Johnson, R.C., Kwon, D.S., Addo, M.M., Brumme, C., Routy, J.-P., Little, S., et al. (2009). Human immunodeficiency virus type 1-specific CD8+ T-cell responses during primary infection are major determinants of the viral set point and loss of CD4+ T cells. J. Virol. 83, 7641-7648.

Teixeira, A., Benckhuijsen, W.E., de Koning, P.E., Valentijn, A.R.P.M., and Drijfhout, J.W. (2002). The use of DODT as a non-malodorous scavenger in Fmoc-based peptide synthesis. Protein Pept. Lett. 9, 379-385.

Tarke, A., Sidney, J., Kidd, C.K., Dan, J.M., Ramirez, S.I., Yu, E.D., Mateus, J., da Silva Antunes, R., Moore, E., Rubiro, P., et al. (2021). Comprehensive analysis of T cell immunodominance and immunoprevalence of SARS-CoV-2 epitopes in COVID-19 cases. Cell Rep Med 2, 100204.

Weisblum, Y., Schmidt, F., Zhang, F., DaSilva, J., Poston, D., Lorenzi, J.C.C., Muecksch, F., Rutkowska, M., Hoffmann, H.-H., Michailidis, E., et al. (2020). Escape from neutralizing antibodies by SARS-CoV-2 spike protein variants. BioRxiv.

Having described the invention we claim:

1. A multi-epitope T cell immunogen composition comprising two or more highly networked Coronavirus CTL epitopes, wherein the two or more highly networked Coronavirus CTL epitopes each have a network score of at least about 3.0.

2. The multi-epitope T cell immunogen composition of claim 1, wherein the two or more highly networked coronavirus CTL epitopes are selected from among the highly networked Coronavirus CTL epitopes in Table 5.

3. The multi-epitope T cell immunogen composition of claim 1, wherein at least one of the two or more highly networked coronavirus CTL epitopes is a variant having at least about 65% to about 99% homology to a highly networked Coronavirus CTL epitope in Table 5.

4. The multi-epitope T cell immunogen composition of claim 2, wherein at least one of the highly networked Coronavirus CTL epitopes is an epitope having the amino acid sequence of AGEAANFCAL (SEQ ID NO: 1), ALNTLVKQL (SEQ ID NO: 2), AMPNMLRIM (SEQ ID NO: 3), APGTAVLRQW (SEQ ID NO: 4), APSASAFF (SEQ ID NO: 5), APSASAFFGM (SEQ ID NO: 6), AQFAPSASA (SEQ ID NO: 7), AQVLSEMVM (SEQ ID NO: 8), ARTRSMWSF (SEQ ID NO: 9), AWPLIVTAL (SEQ ID NO: 10), DRAMPNML (SEQ ID NO: 11), FCYMHHMEL (SEQ ID NO: 12), FELLHAPATV (SEQ ID NO: 13), FPQSAPHGV (SEQ ID NO: 14), FPQSAPHGVVF (SEQ ID NO: 15), GEAANFCAL (SEQ ID NO: 16), GHLRIAGHHL (SEQ ID NO: 17), GNYQCGHYK (SEQ ID NO: 18), GTAVLRQW (SEQ ID NO: 19), GVDIAANTVIW (SEQ ID NO: 20), GVFVSNGTHW (SEQ ID NO: 21), IAANTVIW (SEQ ID NO: 22), ILPVSMTK (SEQ ID NO: 23), IPTITQMNL (SEQ ID NO: 24), IPYNSVTSSI (SEQ ID NO: 25), IYQTSNFRV (SEQ ID NO: 26), KGIYQTSNF (SEQ ID NO: 27), KGIYQTSNFR (SEQ ID NO: 28), KLNDLCFTNV (SEQ ID NO: 29), KLNDLCFTNVY (SEQ ID NO: 30), KQASLNGVTL (SEQ ID NO: 31), KRNVIPTITQM (SEQ ID NO: 32), KRVDFCGK (SEQ ID NO: 33), KRVDFCGKGY (SEQ ID NO: 34), KTSVDCTMY (SEQ ID NO: 35), KWADNNCYL (SEQ ID NO: 36), LLKSAYENF (SEQ ID NO: 37), LLTLQQIEL (SEQ ID NO: 38), LLYDANYFL (SEQ ID NO: 39), LPVSMTKTSV (SEQ ID NO: 40), LRIAGHHL (SEQ ID NO: 41), LRQWLPTGTL (SEQ ID NO: 42), LRQWLPTGTLL (SEQ ID NO: 43), MIAQYTSAL (SEQ ID NO: 44), MPILTLTRAL (SEQ ID NO: 45), MVMCGGSLY (SEQ ID NO: 46), MVMCGGSLYV (SEQ ID NO: 47), MWSFNPETNIL (SEQ ID NO: 48), NASSSEAFL (SEQ ID NO: 49), NPLLYDANYFL (SEQ ID NO: 50), NSSPDDQIGY (SEQ ID NO: 51), NSSPDDQIGYY (SEQ ID NO: 52), NVIPTITQM (SEQ ID NO: 53), PDDQIGYY (SEQ ID NO: 54), PGTAVLRQW (SEQ ID NO: 55), PLLTDEMIAQY (SEQ ID NO: 56), QFAPSASAF (SEQ ID NO: 57), QFAPSASAFF (SEQ ID NO: 58), QPGQTFSVL (SEQ ID NO: 59), QPTESIVRF (SEQ ID NO: 60), QTFSVLACY (SEQ ID NO: 61), QVNGLTSIKW (SEQ ID NO: 62), QWLPTGTLL (SEQ ID NO: 63), RGVYYPDKVF (SEQ ID NO: 64), RLFARTRSMW (SEQ ID NO: 65), RQLLFVVEV (SEQ ID NO: 66), RQWLPTGTL (SEQ ID NO: 67), RQWLPTGTLL (SEQ ID NO: 68), RRGPEQTQGNF (SEQ ID NO: 69), RTRSMWSF (SEQ ID NO: 70), RVIHFGAGSDK (SEQ ID NO: 71), RVQPTESIVRF (SEQ ID NO: 72), SALNHTKKW (SEQ ID NO: 73), SEMVMCGGSL (SEQ ID NO: 74), SEYTGNYQC (SEQ ID NO: 75), SFNPETNIL (SEQ ID NO: 76), SFNPETNILL (SEQ ID NO: 77), SIKNFKSVL (SEQ ID NO: 78), SIKWADNNCY (SEQ ID NO: 79), SMWSFNPET (SEQ ID NO: 80), SPDDQIGYY (SEQ ID NO: 81), SSPDDQIGYY (SEQ ID NO: 82), TEILPVSM (SEQ ID NO: 83), TILTRPLL (SEQ ID NO: 84), TSNEVAVLY (SEQ ID NO: 85), TTLPVNVAF (SEQ ID NO: 86), VAPGTAVLRQW (SEQ ID NO: 87), VIPTITQMNL (SEQ ID NO: 88), VLNDILSRL (SEQ ID NO: 89), VMCGGSLYV (SEQ ID NO: 90), VMCGGSLYVK (SEQ ID NO: 91), VNGLTSIKW (SEQ ID NO: 92), VPVVDSYY (SEQ ID NO: 93), VSMTKTSV (SEQ ID NO: 94), VTANVNALL (SEQ ID NO: 95), VVNAANVYL (SEQ ID NO: 96), YDANYFLCW (SEQ ID NO: 97), YHLMSFPQSA (SEQ ID NO: 98), YLATALLTL (SEQ ID NO: 99), YPKCDRAM (SEQ ID NO: 100), YQCGHYKHI (SEQ ID NO: 101), YQDVNCTEV (SEQ ID NO: 102), YRFNGIGV (SEQ ID NO: 103), YTGNYQCGHY (SEQ ID NO: 104), YYPDKVFRSSV (SEQ ID NO: 105), YYSLLMPIL (SEQ ID NO: 106) or YYSLLMPILTL (SEQ ID NO: 107).

5. The multi-epitope T cell immunogen composition of claim 1, wherein at least one of the two or more highly networked coronavirus CTL epitopes are selected from among the highly networked Coronavirus CTL epitope regions in Table 5 having an amino acid sequence of ALNTLVKQL (SEQ ID NO: 2), APSASAFF(SEQ ID NO: 5), APSASAFFGM (SEQ ID NO: 6), AQFAPSASA (SEQ ID NO: 7), ARTRSMWSF (SEQ ID NO: 9), AWPLIVTAL (SEQ ID NO: 10), FELLHAPATV (SEQ ID NO: 13), FPQSAPHGV (SEQ ID NO: 14), FPQSAPHGVVF (SEQ ID NO: 15), GHLRIAGHHL (SEQ ID NO: 17), GVFVSNGTHW (SEQ ID NO: 21), ILPVSMTK (SEQ ID NO: 23), IPYNSVTSSI (SEQ ID NO: 25), IYQTSNFRV (SEQ ID NO: 26), KGIYQTSNF (SEQ ID NO: 27), KGIYQTSNFR (SEQ ID NO: 28), KLNDLCFTNV (SEQ ID NO: 29), KLNDLCFTNVY (SEQ ID NO: 30), KRVDFCGK (SEQ ID NO: 33), KRVDFCGKGY (SEQ ID NO: 34), KTSVDCTMY (SEQ ID NO: 35), LLYDANYFL (SEQ ID NO: 39), LPVSMTKTSV (SEQ ID NO: 40), LRIAGHHL (SEQ ID NO: 41), MIAQYTSAL (SEQ ID NO: 44), MPILTLTRAL (SEQ ID NO: 45), MVMCGGSLY (SEQ ID NO: 46), MVMCGGSLYV (SEQ ID NO: 47), MWSFNPETNIL (SEQ ID NO: 48), NASSSEAFL (SEQ ID NO: 49), NPLLYDANYFL (SEQ ID NO: 50), NSSPDDQIGY (SEQ ID NO: 51), NSSPDDQIGYY (SEQ ID NO: 52), PDDQIGYY (SEQ ID NO: 54), PLLTDEMIAQY (SEQ ID NO: 56), QFAPSASAF (SEQ ID NO: 57), QFAPSASAFF (SEQ ID NO: 58), QPTESIVRF (SEQ ID NO: 60), RGVYYPDKVF (SEQ ID NO: 64), RLFARTRSMW (SEQ ID NO: 65), RRGPEQTQGNF (SEQ ID NO: 69), RTRSMWSF (SEQ ID NO: 70), SFNPETNIL (SEQ ID NO: 76), SFNPETNILL (SEQ ID NO: 77), SMWSFNPET (SEQ ID NO: 80), SPDDQIGYY (SEQ ID NO: 81), SSPDDQIGYY (SEQ ID NO: 82), SSPDDQIGYY (SEQ ID NO: 82), TEILPVSM (SEQ ID NO: 83), TILTRPLL (SEQ ID NO: 84), TSNEVAVLY (SEQ ID NO: 85), VLNDILSRL (SEQ ID NO: 89), VSMTKTSV (SEQ ID NO: 94), YDANYFLCW (SEQ ID NO: 97), YHLMSFPQSA (SEQ ID NO: 98), YQDVNCTEV (SEQ ID NO: 102), YRFNGIGV (SEQ ID NO: 103) or YYPDKVFRSSV (SEQ ID NO: 105).

6. The multi-epitope T cell immunogen composition of claim 1, wherein at least one of the two or more highly networked coronavirus CTL epitopes are selected from among the highly networked Coronavirus CTL epitope regions in Table 6 having an amino acid sequence of ALNTLVKQL (SEQ ID NO: 2), APSASAFF(SEQ ID NO: 5), APSASAFFGM (SEQ ID NO: 6), AQFAPSASA (SEQ ID NO: 7), ARTRSMWSF (SEQ ID NO: 9), AWPLIVTAL (SEQ ID NO: 10), FELLHAPATV (SEQ ID NO: 13), FPQSAPHGV (SEQ ID NO: 14), FPQSAPHGVVF (SEQ ID NO: 15), GHLRIAGHHL (SEQ ID NO: 17), GVFVSNGTHW (SEQ ID NO: 21), ILPVSMTK (SEQ ID NO: 23), IPYNSVTSSI (SEQ ID NO: 25), IYQTSNFRV (SEQ ID NO: 26), KGIYQTSNF (SEQ ID NO: 27), KGIYQTSNFR (SEQ ID NO: 28), KLNDLCFTNV (SEQ ID NO: 29), KLNDLCFTNVY (SEQ ID NO: 30), KRVDFCGK (SEQ ID NO: 33), KRVDFCGKGY (SEQ ID NO: 34), KTSVDCTMY (SEQ ID NO: 35), LLYDANYFL (SEQ ID NO: 39), LPVSMTKTSV (SEQ ID NO: 40), LRIAGHHL (SEQ ID NO: 41), MIAQYTSAL (SEQ ID NO: 44), MPILTLTRAL (SEQ ID NO: 45), MVMCGGSLY (SEQ ID NO: 46), MVMCGGSLYV (SEQ ID NO: 47), MWSFNPETNIL (SEQ ID NO: 48), NASSSEAFL (SEQ ID NO: 49), NPLLYDANYFL (SEQ ID NO: 50), NSSPDDQIGY (SEQ ID NO: 51), NSSPDDQIGYY (SEQ ID NO: 52), PDDQIGYY (SEQ ID NO: 54), PLLTDEMIAQY (SEQ ID NO: 56), QFAPSASAF (SEQ ID NO: 57), QFAPSASAFF (SEQ ID NO: 58), QPTESIVRF (SEQ ID NO: 60), RGVYYPDKVF (SEQ ID NO: 64), RLFARTRSMW (SEQ ID NO: 65), RRGPEQTQGNF (SEQ ID NO: 69), RTRSMWSF (SEQ ID NO: 70), SFNPETNIL (SEQ ID NO: 76), SFNPETNILL (SEQ ID NO: 77), SMWSFNPET (SEQ ID NO: 80), SPDDQIGYY (SEQ ID NO: 81), SSPDDQIGYY (SEQ ID NO: 82), SSPDDQIGYY (SEQ ID NO: 82), TEILPVSM (SEQ ID NO: 83), TILTRPLL (SEQ ID NO: 84), TSNEVAVLY (SEQ ID NO: 85), VLNDILSRL (SEQ ID NO: 89), VSMTKTSV (SEQ ID NO: 94), YDANYFLCW (SEQ ID NO: 97), YHLMSFPQSA (SEQ ID NO: 98), YQDVNCTEV (SEQ ID NO: 1 02), YRFNGIGV (SEQ ID NO: 103) or YYPDKVFRSSV (SEQ ID NO: 105).

7. A vector comprising a multi-epitope T cell immunogen, wherein the vector comprises a sequence encoding two or more highly networked Coronavirus CTL epitopes, wherein the two or more highly networked Coronavirus CTL epitopes each have a network score of at least about 3.0.

8. The vector of claim 7, wherein the two or more highly networked coronavirus CTL epitopes are selected from among the highly networked Coronavirus CTL epitopes in Table 5.

9. The vector of claim 7, wherein at least one of the two or more highly networked coronavirus CTL epitopes is a variant having at least about 65% to about 99% homology to a highly networked Coronavirus CTL epitope in Table 5.

10. The vector of claim 7, wherein at least one of the highly networked Coronavirus CTL epitopes is an epitope having an amino acid sequence of AGEAANFCAL, ALNTLVKQL, AMPNMLRIM, APGTAVLRQW, APSASAFF, APSASAFFGM, AQFAPSASA, AQVLSEMVM, ARTRSMWSF, AWPLIVTAL, DRAMPNML, FCYMHHA4EL, FELLHAPATV, FPQSAPHGV, FPQSAPHGVVF, GEAANFCAL, GHLRIAGHHL, GNYQCGHYK, GTAVLRQW, GVDIAANTVIW, GVFVSNGTHW, IAANTVIW, ILPVSMTK, IPTITQMNL, IPYNSVTSSI, IYQTSNFRV, KGIYQTSNF, KGIYQTSNFR, KLNDLCFTNV, KLNDLCFTNVY, KQASLNGVTL, KRNVIPTITQM, KRVDFCGK, KRVDFCGKGY, KTSVDCTMY, KWADNNCYL, LLKSAYENF, LLTLQQIEL, LLYDANYFL, LPVSMTKTSV, LRIAGHHL, LRQWLPTGTL, LRQWLPTGTLL, MIAQYTSAL, MPILTLTRAL, MVMCGGSLY, MVMCGGSLYV, MWSFNPETNIL, NASSSEAFL, NPLLYDANYFL, NSSPDDQIGY, NSSPDDQIGYY, NVIPTITQM, PDDQIGYY, PGTAVLRQW, PLLTDEMIAQY, QFAPSASAF, QFAPSASAFF, QPGQTFSVL, QPTESIVRF, QTFSVLACY, QVNGLTSIKW, QWLPTGTLL, RGVYYPDKVF, RLFARTRSMW, RQLLFVVEV, RQWLPTGTL, RQWLPTGTLL, RRGPEQTQGNF, RTRSMWSF, RVIHFGAGSDK, RVQPTESIVRF, SALNHTKKW, SEMVMCGGSL, SEYTGNYQC, SFNPETNIL, SFNPETNILL, SIKNFKSVL, SIKWADNNCY, SMWSFNPET, SPDDQIGYY, SSPDDQIGYY, SSPDDQIGYY, TEILPVSM, TILTRPLL, TSNEVAVLY, TTLPVNVAF, VAPGTAVLRQW, VIPTITQMNL, VLNDILSRL, VMCGGSLYV, VMCGGSLYVK, VNGLTSIKW, VPVVDSYY, VSMTKTSV, VTANVNALL, VVNAANVYL, YDANYFLCW, YHLMSFPQSA, YLATALLTL, YPKCDRAM, YQCGHYKHI, YQDVNCTEV, YRFNGIGV, YTGNYQCGHY, YYPDKVFRSSV, YYSLLMPIL or YYSLLMPILTL.

11. The vector of claim 7, wherein at least one of the two or more highly networked coronavirus CTL epitopes are selected from among the highly networked Coronavirus CTL epitope regions in Table 5 having an amino acid sequence of ALNTLVKQL, APSASAFF, APSASAFFGM, AQFAPSASA, ARTRSMWSF, AWPLIVTAL, FELLHAPATV, FPQSAPHGV, FPQSAPHGVVF, GHLRIAGHHL, GVFVSNGTHW, ILPVSMTK, IPYNSVTSSI, IYQTSNFRV, KGIYQTSNF, KGIYQTSNFR, KLNDLCFTNV, KLNDLCFTNVY, KRVDFCGK, KRVDFCGKGY, KTSVDCTMY, LLYDANYFL, LPVSMTKTSV, LRIAGHHL, MIAQYTSAL, MPILTLTRAL, MVMCGGSLY, MVMCGGSLYV, MWSFNPETNIL, NASSSEAFL, NPLLYDANYFL, NSSPDDQIGY, NSSPDDQIGYY, PDDQIGYY, PLLTDEMIAQY, QFAPSASAF, QFAPSASAFF, QPTESIVRF, RGVYYPDKVF, RLFARTRSMW, RRGPEQTQGNF, RTRSMWSF, SFNPETNIL, SFNPETNILL, SMWSFNPET, SPDDQIGYY, SSPDDQIGYY, SSPDDQIGYY, TEILPVSM, TILTRPLL, TSNEVAVLY, VLNDILSRL, VSMTKTSV, YDANYFLCW, YHLMSFPQSA, YQDVNCTEV, YRFNGIGV or YYPDKVFRSSV.

12. The vector of claim 7, wherein at least one of the two or more highly networked coronavirus CTL epitopes are selected from among the highly networked Coronavirus CTL epitope regions in Table 6 having an amino acid sequence of ALNTLVKQL, APSASAFF, APSASAFFGM, AQFAPSASA, ARTRSMWSF, AWPLIVTAL, FELLHAPATV, FPQSAPHGV, FPQSAPHGVVF, GHLRIAGHHL, GVFVSNGTHW, ILPVSMTK, IPYNSVTSSI, IYQTSNFRV, KGIYQTSNF, KGIYQTSNFR, KLNDLCFTNV, KLNDLCFTNVY, KRVDFCGK, KRVDFCGKGY, KTSVDCTMY, LLYDANYFL, LPVSMTKTSV, LRIAGHHL, MIAQYTSAL, MPILTLTRAL, MVMCGGSLY, MVMCGGSLYV, MWSFNPETNIL, NASSSEAFL, NPLLYDANYFL, NSSPDDQIGY, NSSPDDQIGYY, PDDQIGYY, PLLTDEMIAQY, QFAPSASAF, QFAPSASAFF, QPTESIVRF, RGVYYPDKVF, RLFARTRSMW, RRGPEQTQGNF, RTRSMWSF, SFNPETNIL, SFNPETNILL, SMWSFNPET, SPDDQIGYY, SSPDDQIGYY, SSPDDQIGYY, TEILPVSM, TILTRPLL, TSNEVAVLY, VLNDILSRL, VSMTKTSV, YDANYFLCW, YHLMSFPQSA, YQDVNCTEV, YRFNGIGV or YYPDKVFRSSV.

13. The vector of claim 7, wherein, for each of the highly networked Coronavirus CTL epitopes, the vector comprises an endoplasmic reticulum insertion signal sequence (ERISS) and/or sequence encoding a pan HLA DR-binding epitope (PADRE).

14. The vector of claim 7, wherein, for each of the highly networked Coronavirus CTL epitopes, the vector comprises the natural N-terminal and C-terminal flanking amino acid sequences for each epitope up to 30 amino acids as delineated in NCBI sequence Accession #: NC_045512.

15. The vector of claim 7, wherein, for each of the highly networked Coronavirus CTL epitopes, the vector comprises an enzyme cleavage site sequence.

16. The vector of claim 15, wherein the enzyme cleavage site is a furin cleavage site sequence.

17. The vector of claim 7, wherein the sequences encoding the highly networked Coronavirus CTL epitopes are directly linked to each other.

18. The vector of claim 7, wherein the sequences encoding the two or more highly networked Coronavirus CTL epitopes are linked by a linker sequence.

19. The vector of claim 18, wherein the linker sequence comprises Alanine and Tyrosine.

20. The vector of claim 18, wherein the linker sequence comprises Glycine and Proline.

21. The vector of claim 9, wherein the percent homology to the epitope sequence is 75% to 85%.

22. A pharmaceutical composition comprising the vector of any one of claims 7 to 21.

23. A method of preventing or treating a COVID infection in a subject, said method comprising administering the vector of any one of claims 7 to 21 to the subject.

24. A cell expressing the vector of any one of claims 7 to 21.

25. The cell of claim 24, wherein the cell is an antigen presenting cell.

26. The cell of claim 24 and 25, wherein the cell is a human cell.

27. The cell of any one of claims 24 to 26, wherein the highly networked Coronavirus CTL epitopes are restricted by one or more HLA alleles.

28. The cell of any one of claims 24 to 27, wherein the cell is obtained from a subject diagnosed with COVID.

29. A composition comprising any one of the cells of claims 24-28.

30. A method comprising administering to a subject the cell of any one of claims 24 to 28 or the composition of claim 29.

31. A polypeptide comprising two or more highly networked Coronavirus CTL epitopes, wherein the two or more highly networked Coronavirus CTL epitopes each have a network score of at least about 3.0.

32. A cell expressing the polypeptide of claim 31.

33. An exosome comprising the polypeptide of claim 31.

34. A method comprising engineering a human cell to comprise, on its surface, at least two or more highly networked Coronavirus CTL epitopes each having a network score of at least about 3.0 and administering the engineered cell to a subject.

35. The method of claim 34, wherein the highly networked Coronavirus CTL epitopes are restricted on the surface of the cell by one or more HLA alleles.

36. The method of claim 34, wherein the cell is an antigen presenting cell.

37. The method of claim 34, wherein the cell is a human cell.

38. A method comprising administering a vector expressing at least two or more highly networked Coronavirus CTL epitopes each having a network score of at least about 3.0 and administering the vector to the subject.

39. The method of claim 38, wherein upon expression of the vector in a cell the highly networked Coronavirus CTL epitopes are restricted on the surface of the cell by one or more HLA alleles.

40. The method of claim 38, wherein the cell is an antigen presenting cell.

41. The method of claim 38, wherein the cell is a human cell.

42. A method comprising:
selecting two or more Coronavirus CTL epitopes from an Coronavirus proteome, that have a network score that meets a threshold value, the network score for a given epitope being determinable by generating at least one network representing protein structure, calculating a set of network parameters, combining the network parameters to determine a network score for each amino acid residue in the protein structure, and generating the network score for each of a plurality of epitopes as a weighted linear combination of the respective network scores for the amino acid residues of the epitopes; and administering to the subject a therapeutically effective amount of a T cell immunogen composition and a pharmaceutically acceptable carrier, the T cell immunogen composition including the two or more selected Coronavirus CTL epitopes.

43. The method of claim 42, wherein the threshold value is such that the selected two or more Coronavirus CTL epitopes each have a network score of at least about 3.0.

44. The method of claim 42, wherein the selected two or more highly networked coronavirus CTL epitopes are selected from the highly networked Coronavirus CTL epitopes in Table 5.

45. The method of claim 44, wherein the selected two or more highly networked Coronavirus CTL epitopes have an amino acid sequence of AGEAANFCAL, ALNTLVKQL, AMPNMLRIM, APGTAVLRQW, APSASAFF, APSASAFFGM, AQFAPSASA, AQVLSEMVM, ARTRSMWSF, AWPLIVTAL, DRAMPNML, FCYMHHMEL, FELLHAPATV, FPQSAPHGV, FPQSAPHGVVF, GEAANFCAL, GHLRIAGHHL, GNYQCGHYK, GTAVLRQW, GVDIAANTVIW, GVFVSNGTHW, IAANTVIW, ILPVSMTK, IPTITQMNL, IPYNSVTSSI, IYQTSNFRV, KGIYQTSNF, KGIYQTSNFR, KLNDLCFTNV, KLNDLCFTNVY, KQASLNGVTL, KRNVIPTITQM, KRVDFCGK, KRVDFCGKGY, KTSVDCTMY, KWADNNCYL, LLKSAYENF, LLTLQQIEL, LLYDANYFL, LPVSMTKTSV, LRIAGHHL, LRQWLPTGTL, LRQWLPTGTLL, MIAQYTSAL, MPILTLTRAL, MVMCGGSLY, MVMCGGSLYV, MWSFNPETNIL, NASSSEAFL, NPLLYDANYFL, NSSPDDQIGY, NSSPDDQIGYY, NVIPTITQM, PDDQIGYY, PGTAVLRQW, PLLTDEMIAQY, QFAPSASAF, QFAPSASAFF, QPGQTFSVL, QPTESIVRF, QTFSVLACY, QVNGLTSIKW, QWLPTGTLL, RGVYYPDKVF, RLFARTRSMW, RQLLFVVEV, RQWLPTGTL, RQWLPTGTLL, RRGPEQTQGNF, RTRSMWSF, RVIHFGAGSDK, RVQPTESIVRF, SALNHTKKW, SEMVMCGGSL, SEYTGNYQC, SFNPETNIL, SFNPETNILL, SIKNFKSVL, SIKWADNNCY, SMWSFNPET, SPDDQIGYY, SSPDDQIGYY, SSPDDQIGYY, TEILPVSM, TILTRPLL, TSNEVAVLY, TTLPVNVAF, VAPGTAVLRQW, VIPTITQMNL, VLNDILSRL, VMCGGSLYV, VMCGGSLYVK, VNGLTSIKW, VPVVDSYY, VSMTKTSV, VTANVNALL, VVNAANVYL, YDANYFLCW, YHLMSFPQSA, YLATALLTL, YPKCDRAM, YQCGHYKHI, YQDVNCTEV, YRFNGIGV, YTGNYQCGHY, YYPDKVFRSSV, YYSLLMPIL or YYSLLMPILTL.

46. The method of claim 42, wherein at least one of the two or more highly networked coronavirus CTL epitopes are selected from among the highly networked Coronavirus CTL epitope regions in Table 5 having an amino acid sequence of ALNTLVKQL, APSASAFF, APSASAFFGM, AQFAPSASA, ARTRSMWSF, AWPLIVTAL, FELLHAPATV, FPQSAPHGV, FPQSAPHGVVF, GHLRIAGHHL, GVFVSNGTHW, ILPVSMTK, IPYNSVTSSI, IYQTSNFRV, KGIYQTSNF, KGIYQTSNFR, KLNDLCFTNV, KLNDLCFTNVY, KRVDFCGK, KRVDFCGKGY, KTSVDCTMY, LLYDANYFL, LPVSMTKTSV, LRIAGHHL, MIAQYTSAL, MPILTLTRAL, MVMCGGSLY, MVMCGGSLYV, MWSFNPETNIL, NASSSEAFL, NPLLYDANYFL, NSSPDDQIGY, NSSPDDQIGYY, PDDQIGYY, PLLTDEMIAQY, QFAPSASAF, QFAPSASAFF, QPTESIVRF, RGVYYPDKVF, RLFARTRSMW, RRGPEQTQGNF, RTRSMWSF, SFNPETNIL, SFNPETNILL, SMWSFNPET, SPDDQIGYY, SSPDDQIGYY, SSPDDQIGYY, TEILPVSM, TILTRPLL, TSNEVAVLY, VLNDILSRL, VSMTKTSV, YDANYFLCW, YHLMSFPQSA, YQDVNCTEV, YRFNGIGV or YYPDKVFRSSV.

47. The method of claim 42, wherein at least one of the two or more highly networked coronavirus CTL epitopes are selected from among the highly networked Coronavirus CTL epitope regions in Table 6 having an amino acid sequence of ALNTLVKQL, APSASAFF, APSASAFFGM, AQFAPSASA, ARTRSMWSF, AWPLIVTAL, FELLHAPATV, FPQSAPHGV, FPQSAPHGVVF, GHLRIAGHHL, GVFVSNGTHW, ILPVSMTK, IPYNSVTSSI, IYQTSNFRV, KGIYQTSNF, KGIYQTSNFR, KLNDLCFTNV, KLNDLCFTNVY, KRVDFCGK, KRVDFCGKGY, KTSVDCTMY, LLYDANYFL, LPVSMTKTSV, LRIAGHHL, MIAQYTSAL, MPILTLTRAL, MVMCGGSLY, MVMCGGSLYV, MWSFNPETNIL, NASSSEAFL, NPLLYDANYFL, NSSPDDQIGY, NSSPDDQIGYY, PDDQIGYY, PLLTDEMIAQY, QFAPSASAF, QFAPSASAFF, QPTESIVRF, RGVYYPDKVF, RLFARTRSMW, RRGPEQTQGNF, RTRSMWSF, SFNPETNIL, SFNPETNILL, SMWSFNPET, SPDDQIGYY, SSPDDQIGYY, SSPDDQIGYY, TEILPVSM, TILTRPLL, TSNEVAVLY, VLNDILSRL, VSMTKTSV, YDANYFLCW, YHLMSFPQSA, YQDVNCTEV, YRFNGIGV or YYPDKVFRSSV.

48. The method of claim 42, wherein the two or more selected Coronavirus CTL epitopes induce de novo cytotoxic T cell responses in the subject.

49. The method of claim 42, the T cell immunogen composition comprising a recombinant vector.

50. The method of claim 42, the T cell immunogen composition comprising a viral vector.

51. The method of claim 50, the viral vector selected from the group consisting of a human adenovirus, a rhesus adenovirus, adeno-associated virus, modified Ankara virus, herpesvirus, and CMV viral vectors.

52. The method of claim 42, the T cell immunogen composition comprising a nucleic acid.

53. The method of claim 52, wherein the nucleic acid is selected from the group consisting of DNA, mRNA and replicon RNA.

54. The method of claim 52, wherein the nucleic acid is loaded into a lipid nanoparticle.

55. The method of claim 42, the T cell immunogen composition comprising a peptide based T cell immunogen composition.

56. The method of claim 55, wherein the peptide is loaded into a lipid nanoparticle.

57. The method of claim 55, wherein the peptide is loaded into dendritic cells.

58. A method of preventing COVID infection in a subject, the method comprising:
selecting two or more Coronavirus CTL epitopes from a Coronavirus proteome, that have a network score that meets a threshold value, the network score for a given epitope being determinable by generating at least one network representing protein structure, calculating a set of network parameters, combining the network parameters to determine a network score for each amino acid residue in the protein structure, and generating the network score for each of a plurality of epitopes as a weighted linear combination of the respective network scores for the amino acid residues of the epitopes; and administering to the subject a prophylactically effective amount of a T cell immunogen composition and a pharmaceutically acceptable carrier, the T cell immunogen composition including the two or more selected Coronavirus CTL epitopes.

59. The method of claim 58, wherein the selected two or more Coronavirus CTL epitopes have a network score of at least about 3.0.

60. The method of claim 58, wherein the selected two or more highly networked coronavirus CTL epitopes are selected from the highly networked Coronavirus CTL epitopes in Table 5.

61. The method of claim 60, wherein at least one of the highly networked Coronavirus CTL epitopes is an epitope having an amino acid sequence of AGEAANFCAL, ALNTLVKQL, AMPNMLRIM, APGTAVLRQW, APSASAFF, APSASAFFGM, AQFAPSASA, AQVLSEMVM, ARTRSMWSF, AWPLIVTAL, DRAMPNML, FCYMHHA4EL, FELLHAPATV, FPQSAPHGV, FPQSAPHGVVF, GEAANFCAL, GHLRIAGHHL, GNYQCGHYK, GTAVLRQW, GVDIAANTVIW, GVFVSNGTHW, IAANTVIW, ILPVSMTK, IPTITQMNL, IPYNSVTSSI, IYQTSNFRV, KGIYQTSNF, KGIYQTSNFR, KLNDLCFTNV, KLNDLCFTNVY, KQASLNGVTL, KRNVIPTITQM, KRVDFCGK, KRVDFCGKGY, KTSVDCTMY, KWADNNCYL, LLKSAYENF, LLTLQQIEL, LLYDANYFL, LPVSMTKTSV, LRIAGHHL, LRQWLPTGTL, LRQWLPTGTLL, MIAQYTSAL, MPILTLTRAL, MVMCGGSLY, MVMCGGSLYV, MWSFNPETNIL, NASSSEAFL, NPLLYDANYFL, NSSPDDQIGY, NSSPDDQIGYY, NVIPTITQM, PDDQIGYY, PGTAVLRQW, PLLTDEMIAQY, QFAPSASAF, QFAPSASAFF, QPGQTFSVL, QPTESIVRF, QTFSVLACY, QVNGLTSIKW, QWLPTGTLL, RGVYYPDKVF, RLFARTRSMW, RQLLFVVEV, RQWLPTGTL, RQWLPTGTLL, RRGPEQTQGNF, RTRSMWSF, RVIHFGAGSDK, RVQPTESIVRF, SALNHTKKW, SEMVMCGGSL, SEYTGNYQC, SFNPETNIL, SFNPETNILL, SIKNFKSVL, SIKWADNNCY, SMWSFNPET, SPDDQIGYY, SSPDDQIGYY, SSPDDQIGYY, TEILPVSM, TILTRPLL, TSNEVAVLY, TTLPVNVAF, VAPGTAVLRQW, VIPTITQMNL, VLNDILSRL, VMCGGSLYV, VMCGGSLYVK, VNGLTSIKW, VPVVDSYY, VSMTKTSV, VTANVNALL, VVNAANVYL, YDANYFLCW, YHLMSFPQSA, YLATALLTL, YPKCDRAM, YQCGHYKHI, YQDVNCTEV, YRFNGIGV, YTGNYQCGHY, YYPDKVFRSSV, YYSLLMPIL and/or YYSLLMPILTL.

62. The method of claim 58, wherein at least one of the two or more highly networked coronavirus CTL epitopes are selected from among the highly networked Coronavirus CTL epitope regions in Table 5 having an amino acid sequence of ALNTLVKQL, APSASAFF, APSASAFFGM, AQFAPSASA, ARTRSMWSF, AWPLIVTAL, FELLHAPATV, FPQSAPHGV, FPQSAPHGVVF, GHLRIAGHHL, GVFVSNGTHW, ILPVSMTK, IPYNSVTSSI, IYQTSNFRV, KGIYQTSNF, KGIYQTSNFR, KLNDLCFTNV, KLNDLCFTNVY, KRVDFCGK, KRVDFCGKGY, KTSVDCTMY, LLYDANYFL, LPVSMTKTSV, LRIAGHHL, MIAQYTSAL, MPILTLTRAL, MVMCGGSLY, MVMCGGSLYV, MWSFNPETNIL, NASSSEAFL, NPLLYDANYFL, NSSPDDQIGY, NSSPDDQIGYY, PDDQIGYY, PLLTDEMIAQY, QFAPSASAF, QFAPSASAFF, QPTESIVRF, RGVYYPDKVF, RLFARTRSMW, RRGPEQTQGNF, RTRSMWSF, SFNPETNIL, SFNPETNILL, SMWSFNPET, SPDDQIGYY, SSPDDQIGYY, SSPDDQIGYY, TEILPVSM, TILTRPLL, TSNEVAVLY, VLNDILSRL, VSMTKTSV, YDANYFLCW, YHLMSFPQSA, YQDVNCTEV, YRFNGIGV or YYPDKVFRSSV.

63. The method of claim 58, wherein at least one of the two or more highly networked coronavirus CTL epitopes are selected from among the highly networked Coronavirus CTL epitope regions in Table 6 having an amino acid sequence of ALNTLVKQL, APSASAFF, APSASAFFGM, AQFAPSASA, ARTRSMWSF, AWPLIVTAL, FELLHAPATV, FPQSAPHGV, FPQSAPHGVVF, GHLRIAGHHL, GVFVSNGTHW, ILPVSMTK, IPYNSVTSSI, IYQTSNFRV, KGIYQTSNF, KGIYQTSNFR, KLNDLCFTNV, KLNDLCFTNVY, KRVDFCGK, KRVDFCGKGY, KTSVDCTMY, LLYDANYFL, LPVSMTKTSV, LRIAGHHL, MIAQYTSAL, MPILTLTRAL, MVMCGGSLY, MVMCGGSLYV, MWSFNPETNIL, NASSSEAFL, NPLLYDANYFL, NSSPDDQIGY, NSSPDDQIGYY, PDDQIGYY, PLLTDEMIAQY, QFAPSASAF, QFAPSASAFF, QPTESIVRF, RGVYYPDKVF, RLFARTRSMW, RRGPEQTQGNF, RTRSMWSF, SFNPETNIL, SFNPETNILL, SMWSFNPET, SPDDQIGYY, SSPDDQIGYY, SSPDDQIGYY, TEILPVSM, TILTRPLL, TSNEVAVLY, VLNDILSRL, VSMTKTSV, YDANYFLCW, YHLMSFPQSA, YQDVNCTEV, YRFNGIGV or YYPDKVFRSSV.

64. The method of claim 58, wherein the selected two or more Coronavirus CTL epitopes induce de novo cytotoxic T cell responses in the subject.

65. The method of claim 58, the T cell immunogen composition comprising a recombinant vector.

66. The method of claim 58, the T cell immunogen composition comprising a viral vector.

67. The method of claim 58, the viral vector selected from the group consisting of a human adenovirus, a rhesus adenovirus, adeno-associated virus, modified Ankara virus, herpesvirus, and CMV viral vectors.

68. The method of claim 58, the T cell immunogen composition comprising a nucleic acid.

69. The method of claim 68, wherein the nucleic acid is selected from the group consisting of DNA, mRNA and replicon RNA.

70. The method of claim 68, wherein the nucleic acid is loaded into a lipid nanoparticle.

71. The method of claim 58, the T cell immunogen composition comprising a peptide based T cell immunogen composition.

72. The method of claim 7 1, wherein the peptide is loaded into a lipid nanoparticle.

73. The method of claim 71, wherein the peptide is loaded into dendritic cells.

74. A method of preventing COVID infection in a subject or reducing the severity thereof, the method comprising:
administering to the subject a prophylactically effective amount of a multi-epitope T cell immunogen composition comprising two or more highly networked Coronavirus CTL epitopes, wherein the two or more highly networked Coronavirus CTL epitopes each have a network score of at least about 3.0, and a pharmaceutically acceptable carrier, thereby preventing COVID infection in the subject or reducing the severity thereof.

75. The method of claim 74, wherein the two or more highly networked coronavirus CTL epitopes are selected from among the highly networked Coronavirus CTL epitopes in Table 5.

76. The method of claim 74, wherein at least one of the two or more highly networked coronavirus CTL epitopes is a variant having at least about 65% to about 99% homology to a highly networked Coronavirus CTL epitope in Table 5.

77. The method of claim 75, wherein at least one of the highly networked Coronavirus CTL epitopes is an epitope having an amino acid sequence of AGEAANFCAL, ALNTLVKQL, AMPNMLRIM, APGTAVLRQW, APSASAFF, APSASAFFGM, AQFAPSASA, AQVLSEMVM, ARTRSMWSF, AWPLIVTAL, DRAMPNML, FCYMHHA4EL, FELLHAPATV, FPQSAPHGV, FPQSAPHGVVF, GEAANFCAL, GHLRIAGHHL, GNYQCGHYK, GTAVLRQW, GVDIAANTVIW, GVFVSNGTHW, IAANTVIW, ILPVSMTK, IPTITQMNL, IPYNSVTSSI, IYQTSNFRV, KGIYQTSNF, KGIYQTSNFR, KLNDLCFTNV, KLNDLCFTNVY, KQASLNGVTL, KRNVIPTITQM, KRVDFCGK, KRVDFCGKGY, KTSVDCTMY, KWADNNCYL, LLKSAYENF, LLTLQQIEL, LLYDANYFL, LPVSMTKTSV, LRIAGHHL, LRQWLPTGTL, LRQWLPTGTLL, MIAQYTSAL, MPILTLTRAL, MVMCGGSLY, MVMCGGSLYV, MWSFNPETNIL, NASSSEAFL, NPLLYDANYFL, NSSPDDQIGY, NSSPDDQIGYY, NVIPTITQM, PDDQIGYY, PGTAVLRQW, PLLTDEMIAQY, QFAPSASAF, QFAPSASAFF, QPGQTFSVL, QPTESIVRF, QTFSVLACY, QVNGLTSIKW, QWLPTGTLL, RGVYYPDKVF, RLFARTRSMW, RQLLFVVEV, RQWLPTGTL, RQWLPTGTLL, RRGPEQTQGNF, RTRSMWSF, RVIHFGAGSDK, RVQPTESIVRF, SALNHTKKW, SEMVMCGGSL, SEYTGNYQC, SFNPETNIL, SFNPETNILL, SIKNFKSVL, SIKWADNNCY, SMWSFNPET, SPDDQIGYY, SSPDDQIGYY, SSPDDQIGYY, TEILPVSM, TILTRPLL, TSNEVAVLY, TTLPVNVAF, VAPGTAVLRQW, VIPTITQMNL, VLNDILSRL, VMCGGSLYV, VMCGGSLYVK, VNGLTSIKW, VPVVDSYY, VSMTKTSV, VTANVNALL, VVNAANVYL, YDANYFLCW, YHLMSFPQSA, YLATALLTL, YPKCDRAM, YQCGHYKHI, YQDVNCTEV, YRFNGIGV, YTGNYQCGHY, YYPDKVFRSSV, YYSLLMPIL or YYSLLMPILTL.

78. The method of claim 74, wherein at least one of the two or more highly networked coronavirus CTL epitopes are selected from among the highly networked Coronavirus CTL epitope regions in Table 5 having an amino acid sequence of ALNTLVKQL, APSASAFF, APSASAFFGM, AQFAPSASA, ARTRSMWSF, AWPLIVTAL, FELLHAPATV, FPQSAPHGV, FPQSAPHGVVF, GHLRIAGHHL, GVFVSNGTHW, ILPVSMTK, IPYNSVTSSI, IYQTSNFRV, KGIYQTSNF, KGIYQTSNFR, KLNDLCFTNV, KLNDLCFTNVY, KRVDFCGK, KRVDFCGKGY, KTSVDCTMY, LLYDANYFL, LPVSMTKTSV, LRIAGHHL, MIAQYTSAL, MPILTLTRAL, MVMCGGSLY, MVMCGGSLYV, MWSFNPETNIL, NASSSEAFL, NPLLYDANYFL, NSSPDDQIGY, NSSPDDQIGYY, PDDQIGYY, PLLTDEMIAQY, QFAPSASAF, QFAPSASAFF, QPTESIVRF, RGVYYPDKVF, RLFARTRSMW, RRGPEQTQGNF, RTRSMWSF, SFNPETNIL, SFNPETNILL, SMWSFNPET, SPDDQIGYY, SSPDDQIGYY, SSPDDQIGYY, TEILPVSM, TILTRPLL, TSNEVAVLY, VLNDILSRL, VSMTKTSV, YDANYFLCW, YHLMSFPQSA, YQDVNCTEV, YRFNGIGV or YYPDKVFRSSV.

79. The method of claim 74, wherein at least one of the two or more highly networked coronavirus CTL epitopes are selected from among the highly networked Coronavirus CTL epitope regions in Table 6 having an amino acid sequence of ALNTLVKQL, APSASAFF, APSASAFFGM, AQFAPSASA, ARTRSMWSF, AWPLIVTAL, FELLHAPATV, FPQSAPHGV, FPQSAPHGVVF, GHLRIAGHHL, GVFVSNGTHW, ILPVSMTK, IPYNSVTSSI, IYQTSNFRV, KGIYQTSNF, KGIYQTSNFR, KLNDLCFTNV, KLNDLCFTNVY, KRVDFCGK, KRVDFCGKGY, KTSVDCTMY, LLYDANYFL, LPVSMTKTSV, LRIAGHHL, MIAQYTSAL, MPILTLTRAL, MVMCGGSLY, MVMCGGSLYV, MWSFNPETNIL, NASSSEAFL, NPLLYDANYFL, NSSPDDQIGY, NSSPDDQIGYY, PDDQIGYY, PLLTDEMIAQY, QFAPSASAF, QFAPSASAFF, QPTESIVRF, RGVYYPDKVF, RLFARTRSMW, RRGPEQTQGNF, RTRSMWSF, SFNPETNIL, SFNPETNILL, SMWSFNPET, SPDDQIGYY, SSPDDQIGYY, SSPDDQIGYY, TEILPVSM, TILTRPLL, TSNEVAVLY, VLNDILSRL, VSMTKTSV, YDANYFLCW, YHLMSFPQSA, YQDVNCTEV, YRFNGIGV or YYPDKVFRSSV.

80. The method of claim 74, wherein the subject is infected with the P.1 Brazil SARS-CoV-2 variant, B.1.351 South African SARS-CoV-2 variant or B.1.17 United Kingdom SARS-CoV-2 variant.

81. A method comprising:
generating at least one network representing protein structure;
calculating a set of network parameters;
combining the network parameters to determine a network score for each amino acid residue in the protein structure;
generating the network score for each of a plurality of epitopes as a weighted linear combination of the respective network scores for the amino acid residues of the epitopes; and
selecting two or more Coronavirus CTL epitopes from a Coronavirus proteome that have a network score that meets a threshold value.

82. The method of claim 81, wherein the threshold value is, such that the selected two or more Coronavirus CTL epitopes each have a network score of at least about 3.0.

83. A multi-epitope T cell immunogen composition comprising highly networked Coronavirus CTL epitopes RGVYYPDKVFRSSV, KGIYQTSNFRVQPTESIVRF, KLNDLCFTNVY, FELLHAPATV, TSNEVAVLYQDVNCTEV, TEILPVSMTKTSVDCTMY, PLLTDEMIAQYTSAL, YRFNGIGV, ALNTLVKQLSSNFGAISSVLNDILSRL, KRVDFCGKGYHLMSFPQSAPHGVVF, GVFVSNGTHW, NPLLYDANYFLCWHTNCYDYCIPYNSVTSSI, RLFARTRSMWSFNPETNILLNVPLHGTILTRPLLESELVIGAVILRGHLRIAGHHL, NSSPDDQIGYY, and RRGPEQTQGNFGDQELIRQGTDYKHWPQIAQFAPSASAFFGM.


SEQUENCE LISTING (107 sequences)

SEQ ID NO: 1 - protein, 10 aa, Artificial Sequence
  Synthetic Construct
AGEAANFCAL

SEQ ID NO: 2 - protein, 9 aa, Artificial Sequence
  Synthetic Construct
ALNTLVKQL

SEQ ID NO: 3 - protein, 9 aa, Artificial Sequence
  Synthetic Construct
AMPNMLRIM

SEQ ID NO: 4 - protein, 10 aa, Artificial Sequence
  Synthetic Construct
APGTAVLRQW

SEQ ID NO: 5 - protein, 8 aa, Artificial Sequence
  Synthetic Construct
APSASAFF

SEQ ID NO: 6 - protein, 10 aa, Artificial Sequence
  Synthetic Construct
APSASAFFGM

SEQ ID NO: 7 - protein, 9 aa, Artificial Sequence
  Synthetic Construct
AQFAPSASA

SEQ ID NO: 8 - protein, 9 aa, Artificial Sequence
  Synthetic Construct
AQVLSEMVM

SEQ ID NO: 9 - protein, 9 aa, Artificial Sequence
  Synthetic Construct
ARTRSMWSF

SEQ ID NO: 10 - protein, 9 aa, Artificial Sequence
  Synthetic Construct
AWPLIVTAL

SEQ ID NO: 11 - protein, 8 aa, Artificial Sequence
  Synthetic Construct
DRAMPNML

SEQ ID NO: 12 - protein, 9 aa, Artificial Sequence
  Synthetic Construct
FCYMHHMEL

SEQ ID NO: 13 - protein, 10 aa, Artificial Sequence
  Synthetic Construct
FELLHAPATV

SEQ ID NO: 14 - protein, 9 aa, Artificial Sequence
  Synthetic Construct
FPQSAPHGV

SEQ ID NO: 15 - protein, 11 aa, Artificial Sequence
  Synthetic Construct
FPQSAPHGVVF

SEQ ID NO: 16 - protein, 9 aa, Artificial Sequence
  Synthetic Construct
GEAANFCAL

SEQ ID NO: 17 - protein, 10 aa, Artificial Sequence
  Synthetic Construct
GHLRIAGHHL

SEQ ID NO: 18 - protein, 9 aa, Artificial Sequence
  Synthetic Construct
GNYQCGHYK

SEQ ID NO: 19 - protein, 8 aa, Artificial Sequence
  Synthetic Construct
GTAVLRQW

SEQ ID NO: 20 - protein, 11 aa, Artificial Sequence
  Synthetic Construct
GVDIAANTVIW

SEQ ID NO: 21 - protein, 10 aa, Artificial Sequence
  Synthetic Construct
GVFVSNGTHW

SEQ ID NO: 22 - protein, 8 aa, Artificial Sequence
  Synthetic Construct
IAANTVIW

SEQ ID NO: 23 - protein, 8 aa, Artificial Sequence
  Synthetic Construct
ILPVSMTK

SEQ ID NO: 24 - protein, 9 aa, Artificial Sequence
  Synthetic Construct
IPTITQMNL

SEQ ID NO: 25 - protein, 10 aa, Artificial Sequence
  Synthetic Construct
IPYNSVTSSI

SEQ ID NO: 26 - protein, 9 aa, Artificial Sequence
  Synthetic Construct
IYQTSNFRV

SEQ ID NO: 27 - protein, 9 aa, Artificial Sequence
  Synthetic Construct
KGIYQTSNF

SEQ ID NO: 28 - protein, 10 aa, Artificial Sequence
  Synthetic Construct
KGIYQTSNFR

SEQ ID NO: 29 - protein, 10 aa, Artificial Sequence
  Synthetic Construct
KLNDLCFTNV

SEQ ID NO: 30 - protein, 11 aa, Artificial Sequence
  Synthetic Construct
KLNDLCFTNVY

SEQ ID NO: 31 - protein, 10 aa, Artificial Sequence
  Synthetic Construct
KQASLNGVTL

SEQ ID NO: 32 - protein, 11 aa, Artificial Sequence
  Synthetic Construct
KRNVIPTITQM

SEQ ID NO: 33 - protein, 8 aa, Artificial Sequence
  Synthetic Construct
KRVDFCGK

SEQ ID NO: 34 - protein, 10 aa, Artificial Sequence
  Synthetic Construct
KRVDFCGKGY

SEQ ID NO: 35 - protein, 9 aa, Artificial Sequence
  Synthetic Construct
KTSVDCTMY

SEQ ID NO: 36 - protein, 9 aa, Artificial Sequence
  Synthetic Construct
KWADNNCYL

SEQ ID NO: 37 - protein, 9 aa, Artificial Sequence
  Synthetic Construct
LLKSAYENF

SEQ ID NO: 38 - protein, 9 aa, Artificial Sequence
  Synthetic Construct
LLTLQQIEL

SEQ ID NO: 39 - protein, 9 aa, Artificial Sequence
  Synthetic Construct
LLYDANYFL

SEQ ID NO: 40 - protein, 10 aa, Artificial Sequence
  Synthetic Construct
LPVSMTKTSV

SEQ ID NO: 41 - protein, 8 aa, Artificial Sequence
  Synthetic Construct
LRIAGHHL

SEQ ID NO: 42 - protein, 10 aa, Artificial Sequence
  Synthetic Construct
LRQWLPTGTL

SEQ ID NO: 43 - protein, 11 aa, Artificial Sequence
  Synthetic Construct
LRQWLPTGTLL

SEQ ID NO: 44 - protein, 9 aa, Artificial Sequence
  Synthetic Construct
MIAQYTSAL

SEQ ID NO: 45 - protein, 10 aa, Artificial Sequence
  Synthetic Construct
MPILTLTRAL

SEQ ID NO: 46 - protein, 9 aa, Artificial Sequence
  Synthetic Construct
MVMCGGSLY

SEQ ID NO: 47 - protein, 10 aa, Artificial Sequence
  Synthetic Construct
MVMCGGSLYV

SEQ ID NO: 48 - protein, 11 aa, Artificial Sequence
  Synthetic Construct
MWSFNPETNIL

SEQ ID NO: 49 - protein, 9 aa, Artificial Sequence
  Synthetic Construct
NASSSEAFL

SEQ ID NO: 50 - protein, 11 aa, Artificial Sequence
  Synthetic Construct
NPLLYDANYFL

SEQ ID NO: 51 - protein, 10 aa, Artificial Sequence
  Synthetic Construct
NSSPDDQIGY

SEQ ID NO: 52 - protein, 11 aa, Artificial Sequence
  Synthetic Construct
NSSPDDQIGYY

SEQ ID NO: 53 - protein, 9 aa, Artificial Sequence
  Synthetic Construct
NVIPTITQM

SEQ ID NO: 54 - protein, 8 aa, Artificial Sequence
  Synthetic Construct
PDDQIGYY

SEQ ID NO: 55 - protein, 9 aa, Artificial Sequence
  Synthetic Construct
PGTAVLRQW

SEQ ID NO: 56 - protein, 11 aa, Artificial Sequence
  Synthetic Construct
PLLTDEMIAQY

SEQ ID NO: 57 - protein, 9 aa, Artificial Sequence
  Synthetic Construct
QFAPSASAF

SEQ ID NO: 58 - protein, 10 aa, Artificial Sequence
  Synthetic Construct
QFAPSASAFF

SEQ ID NO: 59 - protein, 9 aa, Artificial Sequence
  Synthetic Construct
QPGQTFSVL

SEQ ID NO: 60 - protein, 9 aa, Artificial Sequence
  Synthetic Construct
QPTESIVRF

SEQ ID NO: 61 - protein, 9 aa, Artificial Sequence
  Synthetic Construct
QTFSVLACY

SEQ ID NO: 62 - protein, 10 aa, Artificial Sequence
  Synthetic Construct
QVNGLTSIKW

SEQ ID NO: 63 - protein, 9 aa, Artificial Sequence
  Synthetic Construct
QWLPTGTLL

SEQ ID NO: 64 - protein, 10 aa, Artificial Sequence
  Synthetic Construct
RGVYYPDKVF

SEQ ID NO: 65 - protein, 10 aa, Artificial Sequence
  Synthetic Construct
RLFARTRSMW

SEQ ID NO: 66 - protein, 9 aa, Artificial Sequence
  Synthetic Construct
RQLLFVVEV

SEQ ID NO: 67 - protein, 9 aa, Artificial Sequence
  Synthetic Construct
RQWLPTGTL

SEQ ID NO: 68 - protein, 10 aa, Artificial Sequence
  Synthetic Construct
RQWLPTGTLL

SEQ ID NO: 69 - protein, 11 aa, Artificial Sequence
  Synthetic Construct
RRGPEQTQGNF

SEQ ID NO: 70 - protein, 8 aa, Artificial Sequence
  Synthetic Construct
RTRSMWSF

SEQ ID NO: 71 - protein, 11 aa, Artificial Sequence
  Synthetic Construct
RVIHFGAGSDK

SEQ ID NO: 72 - protein, 11 aa, Artificial Sequence
  Synthetic Construct
RVQPTESIVRF

SEQ ID NO: 73 - protein, 9 aa, Artificial Sequence
  Synthetic Construct
SALNHTKKW

SEQ ID NO: 74 - protein, 10 aa, Artificial Sequence
  Synthetic Construct
SEMVMCGGSL

SEQ ID NO: 75 - protein, 9 aa, Artificial Sequence
  Synthetic Construct
SEYTGNYQC

SEQ ID NO: 76 - protein, 9 aa, Artificial Sequence
  Synthetic Construct
SFNPETNIL

SEQ ID NO: 77 - protein, 10 aa, Artificial Sequence
  Synthetic Construct
SFNPETNILL

SEQ ID NO: 78 - protein, 9 aa, Artificial Sequence
  Synthetic Construct
SIKNFKSVL

SEQ ID NO: 79 - protein, 10 aa, Artificial Sequence
  Synthetic Construct
SIKWADNNCY

SEQ ID NO: 80 - protein, 9 aa, Artificial Sequence
  Synthetic Construct
SMWSFNPET

SEQ ID NO: 81 - protein, 9 aa, Artificial Sequence
  Synthetic Construct
SPDDQIGYY

SEQ ID NO: 82 - protein, 10 aa, Artificial Sequence
  Synthetic Construct
SSPDDQIGYY

SEQ ID NO: 83 - protein, 8 aa, Artificial Sequence
  Synthetic Construct
TEILPVSM

SEQ ID NO: 84 - protein, 8 aa, Artificial Sequence
  Synthetic Construct
TILTRPLL

SEQ ID NO: 85 - protein, 9 aa, Artificial Sequence
  Synthetic Construct
TSNEVAVLY

SEQ ID NO: 86 - protein, 9 aa, Artificial Sequence
  Synthetic Construct
TTLPVNVAF

SEQ ID NO: 87 - protein, 11 aa, Artificial Sequence
  Synthetic Construct
VAPGTAVLRQW

SEQ ID NO: 88 - protein, 10 aa, Artificial Sequence
  Synthetic Construct
VIPTITQMNL

SEQ ID NO: 89 - protein, 9 aa, Artificial Sequence
  Synthetic Construct
VLNDILSRL

SEQ ID NO: 90 - protein, 9 aa, Artificial Sequence
  Synthetic Construct
VMCGGSLYV

SEQ ID NO: 91 - protein, 10 aa, Artificial Sequence
  Synthetic Construct
VMCGGSLYVK

SEQ ID NO: 92 - protein, 9 aa, Artificial Sequence
  Synthetic Construct
VNGLTSIKW

SEQ ID NO: 93 - protein, 8 aa, Artificial Sequence
  Synthetic Construct
VPVVDSYY

SEQ ID NO: 94 - protein, 8 aa, Artificial Sequence
  Synthetic Construct
VSMTKTSV

SEQ ID NO: 95 - protein, 9 aa, Artificial Sequence
  Synthetic Construct
VTANVNALL

SEQ ID NO: 96 - protein, 9 aa, Artificial Sequence
  Synthetic Construct
VVNAANVYL

SEQ ID NO: 97 - protein, 9 aa, Artificial Sequence
  Synthetic Construct
YDANYFLCW

SEQ ID NO: 98 - protein, 10 aa, Artificial Sequence
  Synthetic Construct
YHLMSFPQSA

SEQ ID NO: 99 - protein, 9 aa, Artificial Sequence
  Synthetic Construct
YLATALLTL

SEQ ID NO: 100 - protein, 8 aa, Artificial Sequence
  Synthetic Construct
YPKCDRAM

SEQ ID NO: 101 - protein, 9 aa, Artificial Sequence
  Synthetic Construct
YQCGHYKHI

SEQ ID NO: 102 - protein, 9 aa, Artificial Sequence
  Synthetic Construct
YQDVNCTEV

SEQ ID NO: 103 - protein, 8 aa, Artificial Sequence
  Synthetic Construct
YRFNGIGV

SEQ ID NO: 104 - protein, 10 aa, Artificial Sequence
  Synthetic Construct
YTGNYQCGHY

SEQ ID NO: 105 - protein, 11 aa, Artificial Sequence
  Synthetic Construct
YYPDKVFRSSV

SEQ ID NO: 106 - protein, 9 aa, Artificial Sequence
  Synthetic Construct
YYSLLMPIL

SEQ ID NO: 107 - protein, 11 aa, Artificial Sequence
  Synthetic Construct
YYSLLMPILTL